(19) United States
(12) Patent Application Publication
(10) Pub. No.: US 2012/0143282 A1
(43) Pub. Date: Jun. 7, 2012

(54) ELECTRIC STIMULATOR

(75) Inventors: Yoshihito FUKUI, Kanagawa (JP); Takuya Uno, Shizuoka (JP); Masahiro Onada, Kanagawa (JP)

(73) Assignee: Terumo Kabushiki Kaisha, Shibuya-ku (JP)

(21) Appl. No.: 13/365,783

(22) Filed: Feb. 3, 2012

Related U.S. Application Data
(63) Continuation of application No. PCT/JP2010/063269, filed on Aug. 5, 2010.

(30) Foreign Application Priority Data
Aug. 6, 2009 (JP) 2009-183825
Sep. 7, 2009 (JP) 2009-206381
Oct. 14, 2009 (JP) 2009-237436

(51) Int. Cl. A61N 1/372; A61N 1/378

(52) U.S. Cl. 607/48; 607/2; 607/61

(57) ABSTRACT

An electric stimulator includes a stimulation circuit block having a stimulation electrode that stimulates nerves or muscles in a living body and a stimulation circuit that is electrically connected to the stimulation electrode to apply a stimulation signal to the stimulation electrode. Additionally, the electric stimulator further includes a support that is connected to the stimulation circuit block to hold the stimulation electrode at an implantation position in the living body. The stimulation circuit block and the support, except for at least one end of the support, have a shape configured to be inserted into the duct of a tubular insertion tool.

This application is a continuation of International Application No. PCT/JP2010/063269 filed on Aug. 5, 2010, and claims priority to Japanese Application No. 2009-183825 filed on Aug. 6, 2009, Japanese Application No. 2009-206381 filed on Sep. 7, 2009, Japanese Application No. 2009-237436 filed on Oct. 14, 2009, the entire content of each of which is incorporated herein by reference.

### TECHNICAL FIELD

The present invention generally relates to an electric stimulator that electrically stimulates a living body. More specifically, the invention involves an electric stimulator configured to be wholly implanted in a living body, and a method of delivering electric stimulation signals to nerves or muscles at a stimulation site.

### BACKGROUND DISCUSSION

At present, in pain treatment, when conventional medication therapy, nerve block therapy, or surgical therapy do not show any effect or when treatment cannot be continued due to side effects, it has been found that an electric stimulation therapy that electrically stimulates nerves to relieve pain is achieving results. Electrical spinal cord stimulation therapy is one of the electric stimulation therapies and is a stimulation therapy which electrically stimulates the spinal cord, in order to relieve pain that propagates to the brain via the spinal cord.

In the electrical spinal cord stimulation therapy, usually, in order to confirm the validity of pain relief caused by the electric stimulation, a trial period of 24 hours to several weeks is provided. In the trial period, generally, stimulation electrodes are inserted from the back side and placed in an epidural space outside a spinal dura mater that covers the spinal cord, and then, an electrode lead including these stimulation electrodes is connected to an external stimulator outside of the body, and the degree of pain relief is investigated under various stimulation patterns. Implantation of an electric stimulator is not performed in this period. Only when a predetermined effect is seen in this trial period, a long-term implantation (hereinafter referred to as “implantation”) of the electric stimulator is carried out.

When the implantation of the electric stimulator is performed, the electrode lead placed in the trial period is removed, and then new stimulation electrodes are again placed in the epidural space, and an electrode lead including these stimulation electrodes is led to the waist, the abdomen, or the chest through a subcutaneous tunnel. Then, the electrode lead is connected to the electric stimulator and the electric stimulator is implanted subcutaneously therein.

In the trial period in the electrical spinal cord stimulation therapy, the electrode lead is connected to the external stimulator outside the body. Therefore, problems can arise such as risk of infection, restriction of patient activity, and an affect on the judgment of the effectiveness of pain relief since the restriction of the activity acts as stress.

On the other hand, a micro stimulator with no lead including electrodes at both ends of a housing is disclosed in a technique described in U.S. Pat. No. 5,193,539. In this technique, the entire micro stimulator is wholly implanted near nerves, so that the risk of infection can be reduced, and the restriction of the activity of a patient can be reduced as much as possible.

However, when the micro stimulator described in U.S. Pat. No. 5,193,539 is placed in a body cavity, such as an epidural space in the electrical spinal cord stimulation therapy, a problem arises in that it is difficult to place the stimulation electrodes of the stimulator precisely at a desired position, and to continue placing the stimulation electrodes at the position stably for a prolonged period of time. Additionally, there is also a problem in that, once the stimulator is placed in the body cavity, it is difficult to remove the stimulator from the body.

### SUMMARY

The electric stimulator disclosed here is a tubular electric stimulator implantable in a living body. This electric stimulator includes a stimulation electrode(s) that stimulates nerves or muscles in a living body and a stimulation circuit block having a stimulation circuit that is electrically connected to the stimulation electrodes to apply a stimulation signal to the stimulation electrodes. The electric stimulator further includes a support that is connected to the stimulation circuit block to hold the stimulation electrodes at an implantation position in the living body. The stimulation circuit block and the support, except for at least one end of the support, have a size and a shape configured to be inserted into a duct of a tubular insertion tool.

The electric stimulator disclosed here allows stimulation electrodes to be relatively easily and precisely placed at a predetermined position in a body cavity, such as an epidural space, and also allows stimulation electrodes to be placed at the position stably for a relatively prolonged period of time. The electric stimulator can also be relatively easily removed after being placed in a body cavity.

Because the stimulation circuit block and at least a distal portion of the support have a shape insertable into the duct of the tubular insertion tool, a doctor can operate the support from the outside of the body to move the stimulation electrodes to a predetermined position via the tubular insertion tool. Additionally, since the support is connected to the stimulation circuit block, the placing of the stimulation circuit block at the position can be continued by this support. At this time, the electric stimulator can also be removed by pulling the support.

A doctor can operate the support to move the stimulation electrodes to a predetermined position via the tubular insertion tool, such as a tubular needle or a cannula, to place the stimulation electrodes. It is thus relatively easy to move the stimulation electrodes to nerves or muscles to be stimulated, and the stimulation electrodes can he placed at the position stably for a prolonged period of time. The doctor can also operate the support to relatively easily remove the electric stimulator from the body.

Another aspect of the disclosure here involves a rod-shaped electric stimulator implantable in a living body and comprising: a stimulation circuit block having a stimulation electrode exposed at the outer surface of the stimulation electrode to stimulate nerves or muscles in the living body during operational use when the stimulation electrode is positioned in the living body, and a stimulation circuit electrically connected to the stimulation electrode to apply a stimulation signal to the stimulation electrode so that the stimulation electrode stimulates nerves or muscles in the living body. The electric stimulator also includes a support positioned proximally of the stimulation circuit block and connected to the stimulation circuit block, wherein the support possesses a proximal portion positioned outside the living body during operational use when the stimulation electrode is positioned in the living body. The proximal portion of the support is configured to be grasped by a user to maintain the stimulation electrode at an implantation position in the living body, and the stimulation circuit block and the support together are rod-shaped. The entire stimulation circuit block and at least the distal portion of the support are pliable so the stimulation circuit block and the distal portion of the support bend when moved inside the living body during operational use, and the stimulation circuit block and the distal portion of the support possessing a shape and size permitting the stimulation circuit block and the distal portion of the support to be inserted into a duct of a tubular lead-in tool.

Another aspect of the disclosure here involves a method of stimulating nerves or muscles in a living body. The method involves: inserting the distal end of a rod-shaped electric stimulator into a duct of a tubular lead-in tool that is positioned in an epidural space inside a living body, wherein the electric stimulator comprises: a stimulation electrode and a stimulation circuit electrically connected to the stimulation electrode, the stimulation electrode and the stimulation circuit together being connected to a support positioned proximally of the stimulation electrode and the stimulation circuit. The method also involves advancing the electric stimulator through the duct of the tubular lead-in tool by operating a portion of the support to position the stimulation electrode in the epidural space at a stimulation site, and delivering an electric stimulation signal to the electric stimulator to electrically stimulate the stimulation site.

### BRIEF DESCRIPTION OF THE DRAWING FIGURES

FIG. 1 is a perspective view of an overall electric stimulator according to a first embodiment disclosed here.

FIGS. 2A to 2C are exploded external views of the overall electric stimulator of FIG. 1 seen from the top face.

FIG. 3A is an enlarged view of the electric stimulator according to the first embodiment, and FIG. 3B is an axial cross-sectional view of the electric stimulator taken along the section line 3B-3B in FIG. 3A.

FIGS. 4A to 4F are radial cross-sectional views of the electric stimulator according to the first embodiment, with FIG. 4A taken along the section line 4A-4A in FIG. 3A, FIG. 4B taken along the section line 4B-4B in FIG. 3A, FIG. 4C taken along the section line 4C-4C in FIG. 3A, FIG. 4D taken along the section line 4D-4D in FIG. 3A, FIG. 4E taken along the section line 4E-4E in FIG. 3A, and FIG. 4F taken along the section line 4F-4F in FIG. 3A.

FIG. 5 is a block diagram centering on a stimulation circuit according to the first embodiment.

FIG. 6 is an explanatory view of an aspect of a procedure that implants the electric stimulator according to the first embodiment into a living body.

FIG. 7 is an explanatory view of another aspect of procedure, subsequent to FIG. 6, for implanting the electric stimulator according to the first embodiment into the living body.

FIG. 8 is an explanatory view of another aspect of procedure, subsequent to FIG. 7, for implanting the electric stimulator according to the first embodiment into the living body.

FIG. 9 is an explanatory view of another aspect of procedure, subsequent to FIG. 8, for implanting the electric stimulator according to the first embodiment into the living body.

FIG. 10 is an explanatory view of another aspect of procedure, subsequent to FIG. 9, for implanting the electric stimulator according to the first embodiment into the living body.

FIG. 11 is a perspective view of an overall electric stimulator according to a second embodiment disclosed here.

FIGS. 12A to 12C are exploded external views of the overall electric stimulator according to the second embodiment.

FIG. 13A is an enlarged view of the electric stimulator according to the second embodiment, and FIG. 13B is an axial cross-sectional view of the electric stimulator taken along the section line 13B-13B in FIG. 13A.

FIGS. 14A to 14F are radial cross-sectional views of the electric stimulator according to the second embodiment; with FIG. 14A taken along the section line 14A-14A in FIG. 13A, FIG. 14B taken along the section line 14B-14B in FIG. 13A, FIG. 14C taken along the section line 14C-14C in FIG. 13A, FIG. 14D taken along the section line 14D-14D in FIG. 13A, FIG. 14E taken along the section line 14E-14E in FIG. 13A, and FIG. 14F taken along the section line 14F-14F in FIG. 13A.

FIG. 15 is a perspective view of an overall electric stimulator according to a third embodiment.

FIGS. 16A to 16C are exploded external views showing the parts of the electric stimulator shown in FIG. 15 as seen from the top face.

FIG. 17A is an enlarged view showing the electric stimulator according to the third embodiment shown in FIG. 15 as seen from the top face, and FIG. 17B is an axial cross-sectional view of the electric stimulator and its axial internal structure.

FIG. 18 is a perspective view of an overall electric stimulator according to a fourth embodiment.

FIGS. 19A to 19C are exploded external views showing the overall electric stimulator according to the fourth embodiment when the electric stimulator shown in FIG. 18 is seen from the top face.

FIG. 20A is an enlarged view showing the electric stimulator according to the fourth embodiment, and FIG. 20B is an axial cross-sectional view of the electric stimulator taken along the section line 20B-20B in FIG. 20A.

FIGS. 21A to 21F are radial cross-sectional views of the electric stimulator according to the fourth embodiment; with FIG. 21A taken along the section line 21A-21A in FIG. 20A, FIG. 21B taken along the section line 21B-21B in FIG. 20A, FIG. 21C taken along the section line 21C-21C in FIG. 20A, FIG. 21D taken along the section line 21D-21D in FIG. 20A, FIG. 21E taken along the section line 21E-21E in FIG. 20A, and FIG. 21F taken along the section line 21F-21F in FIG. 20A.

FIG. 22 is a perspective view showing an overall electric stimulator according to a fifth embodiment.

FIGS. 23A to 23C are exploded external views of the overall electric stimulator according to the fifth embodiment shown in FIG. 22 as seen from the top face.

FIG. 24A is an enlarged view of the electric stimulator according to the fifth embodiment, and FIG. 24B is an axial cross-sectional view of the electric stimulator taken along the section line 24B-24B in FIG. 24A.

FIGS. 25A to 25F are radial cross-sectional views of the electric stimulator according to the fifth embodiment; with FIG. 25A taken along the section line 25A-25A in FIG. 24A, FIG. 25B taken along the section line 25B-25B in FIG. 24A, FIG. 25C taken along the section line 25C-25C in FIG. 24A, FIG. 25D taken along the section line 25D-25D in FIG. 24A, FIG. 25E taken along the section line 25E-25E in FIG. 24A, and FIG. 25F taken along the section line 25F-25F in FIG. 24A.

FIG. 26 is a perspective view of an overall electric stimulator according to a sixth embodiment.

FIGS. 27A to 27C are exploded external views of the overall electric stimulator according to the sixth embodiment.

FIG. 28A is an explanatory view of a first modification of a support according to the fourth embodiment, and FIG. 28B is an axial cross-sectional view of the support.

FIG. 29A is an explanatory view of a first modification of a support according to the fifth or sixth embodiment, and FIG. 29B is an axial cross-sectional view of the support.

FIG. 30A is an explanatory view of a second modification of the support according to the fourth embodiment, and FIG. 30B is an axial cross-sectional view of the support.

FIG. 31A is an explanatory view of a second modification of the support according to the fifth or sixth embodiment, and FIG. 31B is an axial cross-sectional view of the support.

FIG. 32 is a perspective view of an overall electric stimulator according to a seventh embodiment.

FIGS. 33A to 33D are exploded external views of the overall electric stimulator according to the seventh embodiment.

FIG. 34A is an enlarged view of the electric stimulator according to the seventh embodiment, and FIG. 34B is an axial cross-sectional view of the electric stimulator taken along the section line 34B-34B in FIG. 34A.

FIGS. 35A to 35F are radial cross-sectional views of the electric stimulator according to the seventh embodiment; with FIG. 35A taken along the section line 35A-35A in FIG. 34A, FIG. 35B taken along the section line 35B-35B in FIG. 34A, FIG. 35C taken along the section line 35C-35C in FIG. 34A, FIG. 35D taken along the section line 35D-35D in FIG. 34A, FIG. 35E taken along the section line 35E-35E in FIG. 34A, and FIG. 35F taken along the section line 35F-35F in FIG. 34A.

FIG. 36 is a perspective view of an overall electric stimulator according to an eighth embodiment.

FIGS. 37A to 37D arc exploded external views of the overall electric stimulator according to the eighth embodiment.

FIG. 38A is an enlarged view of the electric stimulator according to the eighth embodiment, and FIG. 38B is an axial cross-sectional view of the electric stimulator taken along the section line 38B-38B in FIG. 38A.

FIGS. 39A to 39F are radial cross-sectional views of the electric stimulator related to the eighth embodiment; with FIG. 39A taken along the section line 39A-39A in FIG. 38A, FIG. 39B taken along the section line 39B-39B in FIG. 38A, FIG. 39C taken along the section line 39C-39C in FIG. 38A, FIG. 39D taken along the section line 39D-39D in FIG. 38A, FIG. 39E taken along the section line 39E-39E in FIG. 38A, and FIG. 39F taken along the section line 39F-39F in FIG. 38A.

FIG. 40 is a perspective view of an overall electric stimulator according to a ninth embodiment.

FIGS. 41A to 41D are exploded external views of the overall electric stimulator according to the ninth embodiment.

FIG. 42A is an enlarged view of the electric stimulator according to the ninth embodiment, and FIG. 42B is an axial cross-sectional view of the electric stimulator taken along the section line 42B-42B in FIG. 42A.

FIGS. 43A to 43D are explanatory views showing modifications of the electric stimulators related to the seventh to ninth embodiment.

FIG. 44 is a perspective view of an overall electric stimulator according to a tenth embodiment.

FIGS. 45A to 45D are exploded external views of the overall electric stimulator according to the tenth embodiment.

FIG. 46A is an enlarged view of the electric stimulator according to the tenth embodiment, and FIG. 46B is an axial cross-sectional view of the electric stimulator according to the tenth embodiment taken along the section line 46B-46B in FIG. 46A.

FIGS. 47A to 47F are radial cross-sectional views of the electric stimulator according to the tenth embodiment; with FIG. 47A taken along the section line 47A-47A in FIG. 46A, FIG. 47B taken along the section line 47B-47B in FIG. 46A, FIG. 47C taken along the section line 47C-47C in FIG. 46A, FIG. 47D taken along the section line 47D-47D in FIG. 46A, FIG. 47E taken along the section line 47E-47E in FIG. 46A, and FIG. 47F taken along the section line 47F-47F in FIG. 46A.

FIG. 48 is a perspective view of an overall electric stimulator according to an eleventh embodiment.

FIGS. 49A to 49D are exploded external views of the overall electric stimulator according to the eleventh embodiment.

FIG. 50A is an enlarged view of the electric stimulator according to the eleventh embodiment, and FIG. 50B is an axial cross-sectional view of the electric stimulator according to the eleventh embodiment taken along the section line 50B-50B.

FIGS. 51A to 51F are radial cross-sectional views of the electric stimulator according to the eleventh embodiment; with FIG. 51 A taken along the section line 51A-51A in FIG. 50A, FIG. 51B taken along the section line 51B-51B in FIG. 50A, FIG. 51C taken along the section line 51C-51C in FIG. 50A, FIG. 51D taken along the section line 51D-51D in FIG. 50A, FIG. 51E taken along the section line 51E-51E in FIG. 50A, and FIG. 51F taken along the section line 51F-51F in FIG. 50A.

FIG. 52 is a perspective view of an overall electric stimulator according to a twelfth embodiment.

FIGS. 53A to 53D are exploded external views of the overall electric stimulator according to the twelfth embodiment.

FIG. 54A is an enlarged view of the electric stimulator according to the twelfth embodiment of the invention, and FIG. 54B is an axial cross-sectional view of the electric stimulator.

### DETAILED DESCRIPTION

Set forth below is a detailed description of embodiments the electric stimulator. The embodiments described below and illustrated in the drawing figures are examples of the electric stimulator. Various technically preferable features and aspects are given, but the scope of the invention is not limited to these embodiments so as long as there is no description indicating otherwise. For example, the following detailed description mentions numerical conditions of respective parameters, but these numerical conditions are merely preferred examples. Dimensions, shapes, and arrangement relations of the respective drawings used for description are also substantial or approximate.

The following description will be set forth generally in the following manner.

### First Embodiment

Configuration of Electric Stimulator

Configuration of Stimulation Circuit or the Like

Implantation Procedure of Electric Stimulator

### Second Embodiment

Configuration of Electric Stimulator

### Third Embodiment

Configuration of Electric Stimulator

### Fourth Embodiment

Configuration of Electric Stimulator

### Fifth Embodiment

Configuration of Electric Stimulator

### Sixth Embodiment

Configuration of Electric Stimulator

### Seventh Embodiment

Configuration of Electric Stimulator

### Eighth Embodiment

Configuration of Electric Stimulator

### Ninth Embodiment

Configuration of Electric Stimulator

### Tenth Embodiment

Configuration of Electric Stimulator

### Eleventh Embodiment

Configuration of Electric Stimulator

### Twelfth Embodiment

Configuration of Electric Stimulator

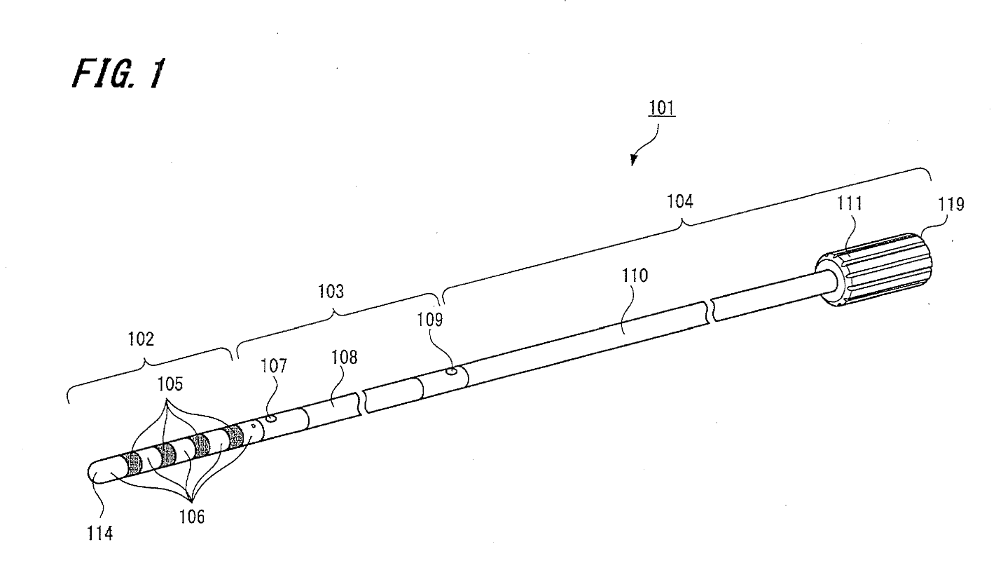

A first embodiment of the electric stimulator disclosed here as an example, is described in detail below with reference to FIGS. 1 to 10.

### Configuration of the Electric Stimulator

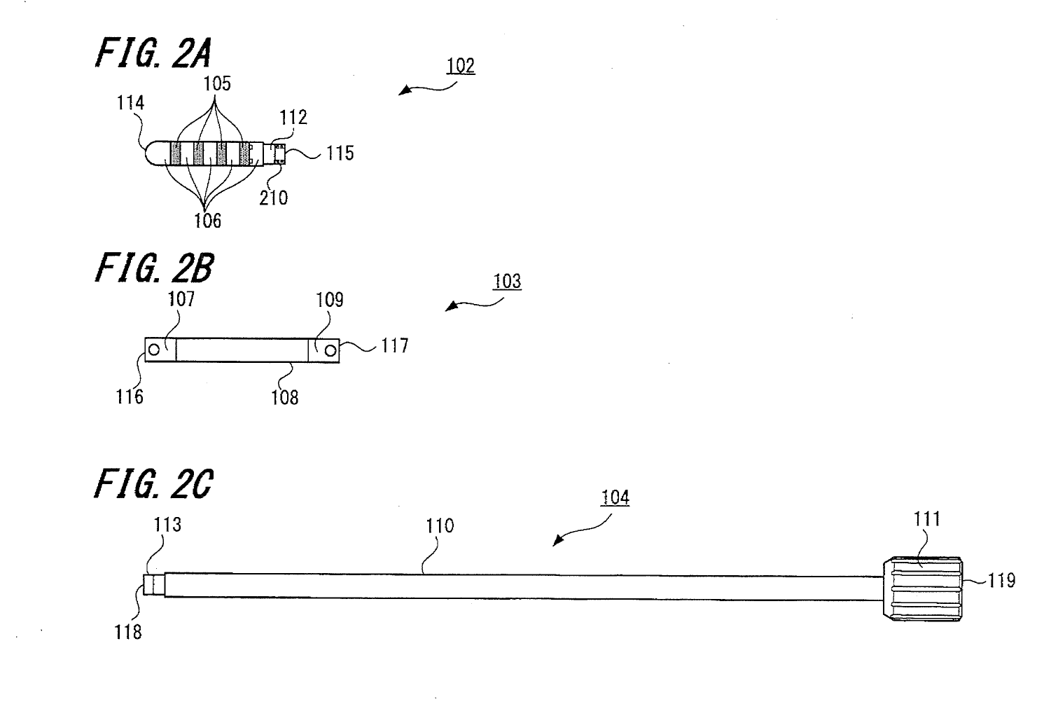

First, the general configuration of an electric stimulator according to the first embodiment is described with reference to FIG. 1 and FIGS. 2A to 2C.

The electric stimulator 101 is formed in the shape of a rod, generates an electric stimulation signal, and stimulates nerves or the like in a living body by the stimulation signal. When the electric stimulator 101 stimulates the nerves of the spinal cord, the electric stimulator 101 is implanted in the living body (for example, an epidural space where the distance between the spinal dura mater and the ligamentum flavum is about 5 mm). Therefore, as for the electric stimulator 101, the diameter from a distal end 114 to a predetermined portion of a support 104 (that will be described below) is preferably about 1 mm to 3 mm.

The electric stimulator 101 is comprised of an electrode block 102, a circuit block 103, and the support 104. The electrode block 102 and the circuit block 103 are attachable and detachable from one another by a connector part 107, and the circuit block 103 and the support 104 are attachable and detachable from one another by a connector part 109. More specifically, as shown in FIGS. 2A to 2C, as for the electrode block 102 and the circuit block 103, a connector part 112 on the electrode block 102 side and the connector part 107 on the circuit block 103 side are fixed together, for example, with a screw or the like. Similarly, as for the circuit block 103 and the support 104, the connector part 109 on the circuit block 103 side and a connector part 113 on the support 104 side are fixed together with a screw or the like.

The distal end 114 of the electrode block 102 is substantially hemispherically shaped, and the other portions are substantially cylindrically shaped. The radius of the substantially hemispherical portion of the distal end 114 is preferably about 0.5 mm to 1.5 mm, and the diameter of the other substantially cylindrical portions is preferably about 1 mm to 3 mm.

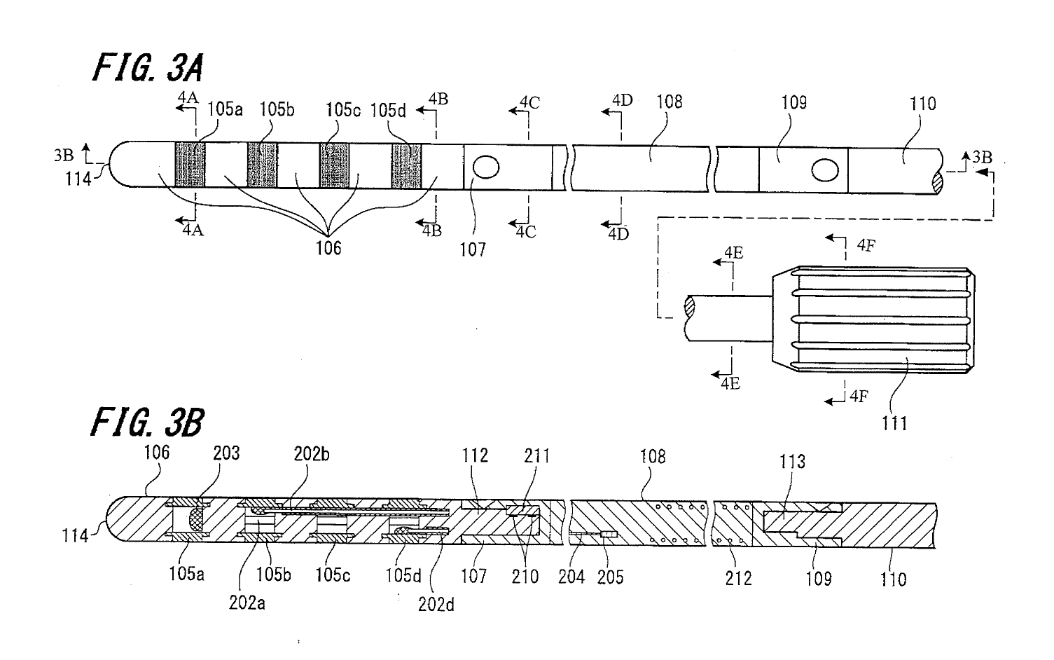

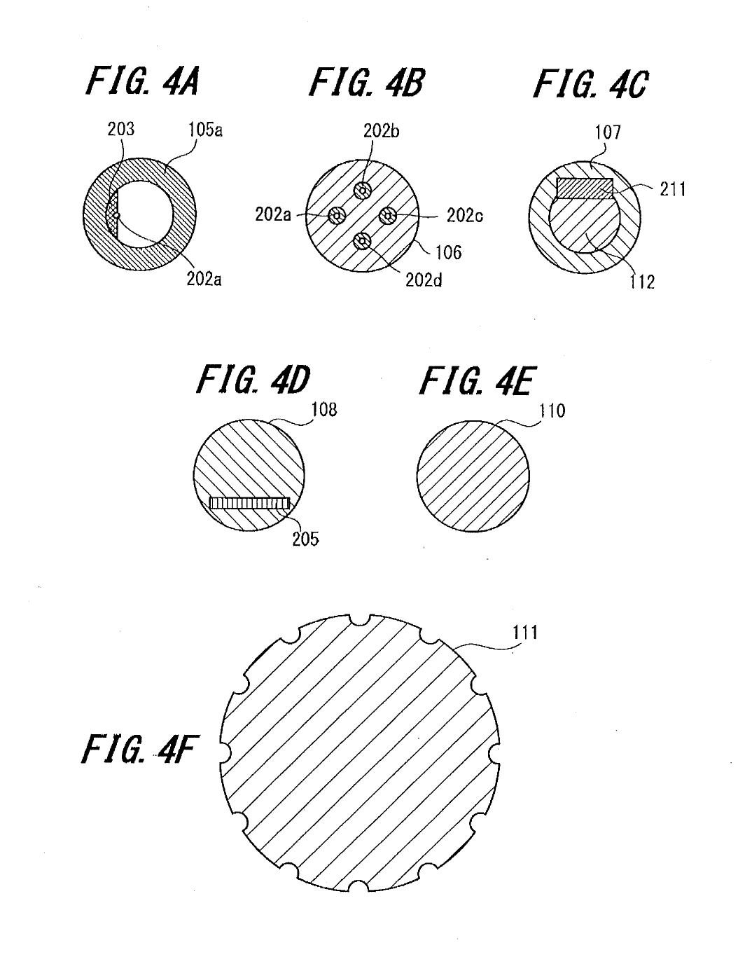

The electrode block 102 includes a plurality of spaced apart stimulation electrodes 105 mounted on a body 106 so that the stimulation electrodes are able to stimulate nerves or the like. The stimulation electrodes are such that when the electric stimulator 101 is implanted in a living body, the respective stimulation electrodes 105 are exposed to the living body. The spaced apart stimulation electrodes mounted on the body result in a plurality of exposed body portions 106, each of which is exposed between axially adjacent stimulation electrodes 105 or at axial ends of the electrode block 102. In the illustrated embodiment, the electrode block 102 includes four stimulation electrodes 105 spaced apart at equal intervals. The electrode block 102 also includes the connector part 112 that connects the proximal end 115 side of the body 106 and a distal end 116 of the circuit block 103 so as to be continuous is included. Though the number of stimulation electrodes 105 is four in the first embodiment, this is merely an example, and the number of the stimulation electrodes 105 can be varied. The internal configuration of the electrode block 102 will be described below in FIGS. 3A and 3B and FIGS. 4A to 4F.

The circuit block 103 is substantially cylindrically shaped with the same outer diameter as the electrode block 102. The circuit block 103 includes the connector part 107 that is connected to the connector part 112 of the electrode block 102 such that the distal end 116 is continuous with the proximal end 115 side of the body 106 of the electrode block 102. Additionally, the circuit block 103 is provided with a body 108 that is continuous with the connector part 107. Moreover, the circuit block 103 includes the connector part 109 that is continuous with a proximal end 117 of the body 108 and connects the proximal end 117 of the circuit block 103 and the support 104. The internal configuration of the circuit block 103 will be described below in FIGS. 3A and 3B and FIGS. 4A to 4F.

The support 104 includes the connector part 113 that is connected to the circuit block 103, a substantially cylindrically shaped body 110 with the same diameter as the electrode block 102, and a substantially cylindrical holder part 111 that has a larger diameter than the body 110.

The connector part 113 of the support 104 is connected to the connector part 109 of the circuit block 103 such that a distal end 118 side of the body 110 is continuous with the circuit block 103. The body 110 is a portion that connects the connector part 113 and the holder part 111 arranged on a proximal end 119 side. In addition, the holder part 111 is a place that is held when a doctor inserts the electric stimulator 101 into a living body. The internal configuration of the support 104 will be described below in FIGS. 3A and 3B and FIGS. 4A to 4F.

Set forth next, with reference to FIGS. 3A, 3B and FIGS. 4A to 4F, is a detailed description of the internal configuration of the electric stimulator 101. First, the internal configuration of the electrode block 102 will be described.

The body 106 is made of resin material(s) that have pliability and biocompatibility, for example, materials such as silicone or polyurethane. The distal end 114 of the body 106 is substantially hemispherical as described above, and the radius of the distal end is preferably within a range of about 0.5 mm to 1.5 mm. Portions other than the distal end 114 of the body 106 are substantially cylindrically shaped of which a portion is hollow in the axial direction. The four stimulation electrodes 105 are fixed to this hollow portion so as to be exposed at the surface of the body 106.

The stimulation electrodes 105 are made of materials that have conductivity and biocompatibility, for example materials such as platinum or platinum alloys (platinum 90%/iridium 10% alloy or the like), and are formed in a substantially hollow cylindrical shape. The external diameter of the stimulation electrodes 105 is approximately equal to the external diameter of the body 106. The four stimulation electrodes 105 are defined as stimulation electrodes 105a, 105b, 105c and 105d sequentially from the stimulation electrode on the distal end 114 side.

One end (the distal end) of each of conducting wires 202a to 202d is bonded to a respective one of the stimulation electrodes 105a to 105d (refer to FIG. 4A) with solder 203, and the other end the proximal end) of each of the conducting wires 202a to 202d is electrically connected to the connector part 112. In addition, locations other than the locations of the conducting wires 202 that are bonded with the solder 203 and the locations of the conducting wires 202 that are electrically connected to the connector part 112 are insulated and coated with PTFE (polytetrafluoroethylene) or ETFE (ethylenetetrafluoroethylene) and are completely embedded in the body 106 (refer to FIG. 4B).

The connector part 112 is formed of the same material as the body 106, and is formed as a cutout part or outer diameter reduce part in which a level difference is provided relative to the external diameter of the substantially cylindrical body 106. That is, the outer dimension of the connector part 112 is smaller than the remainder of the body 106. This cutout part extends over a predetermined distance in the axial direction from the proximal end 115 (refer to FIG. 2A). In addition, this cutout part is planar, four connector pins 210 (refer to FIG. 2A) are arranged on the cutout part so as to be exposed, and each of the conducting wires 202a to 202d is electrically connected to a respective one of the four connector pins 210. Each of the four connector pins 210 is thus electrically connected to a respective one of the stimulation electrodes 105a to 105d.

Set forth next is a description of the internal configuration of the circuit block 103 generally shown in FIG. 2B. The connector part 107 is made of the same material (polyurethane or silicone) as the body 106 except for an electrical connecting portion 211 that will be described below. A hole of almost the same shape and size as the external shape (diameter) of the connector part 112 opens in the axial direction in this connector part 107 so that the connector part 107 can be connected to the connector part 112 of the electrode block 102 (refer to FIG. 4C). The external diameter of this connector part 107 is approximately equal to the external diameter of the body 106. Additionally, the connector part 107 includes the electrical connecting portion 211 that is independently and electrically connected to the four connector pins 210, respectively, when this connector part 107 is connected to the connector part 112 of the electrode block 102. In addition, although the connector part 109 provided on the proximal end 117 (refer to FIG. 2B) side of the circuit block 103 has the same shape and size as the connector part 107 provided on the distal end 116 side, something like the electrical connecting portion 211 is not provided.

The body 108 is continuous with the connector parts 107 and 109 and is made of the same material as the connector parts 107 and 109. The body 108 is substantially cylindrically shaped, and is formed such that the diameter thereof is approximately equal to the external diameter of the connector part 107 of the circuit block 103.

A stimulation circuit 205 that is formed by mounting small parts such as custom ICs on a flexible circuit board, and that generates an electric stimulation signal, and a coil portion 212 that is electrically connected to the stimulation circuit 205 is embedded in the body 108. The coil portion 212 is formed so that the axis of the coil portion 212 extends in the axial direction of the body 108.

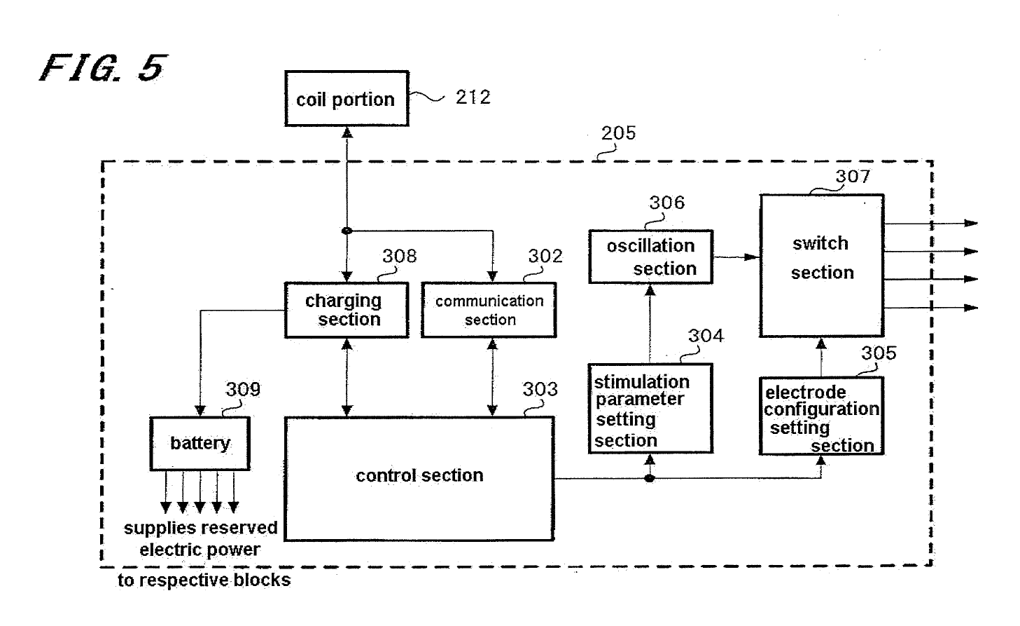

The stimulation circuit 205 is connected to the electrical connecting portion 211 via conducting wires 204 embedded in the body 108 such that the generated electric stimulation signal is independently supplied to the respective stimulation electrodes 105. The electric configuration of the stimulation circuit 205 and the coil portion 212 will be described below in FIG. 5.

Next, the internal configuration of the support 104 generally shown in FIG. 2C will be described. The connector part 113 is made of, for example, polyurethane or silicone and is formed in the same shape and size as the connector part 112 of the electrode block 102 described above. It is noted that means for performing electrical connection with the outside, such as the connector pins 210, may not be provided, different from the connector part 112, since the connector part 109 that is connected to the connector part 113 does not include something like the electrical connecting portion 211.

The body 110 is made of the same material as the connector part 113, and possesses a substantially cylindrical shape. The diameter of the body 110 is approximately equal to the external diameter of the body 108 of the circuit block 103.

The holder part 111 is made of materials such as a plastics, and possesses a substantially cylindrical shape. Since the holder part 111 is a part that is held or grasped when the electric stimulator 101 is inserted into the inside of a body, the external diameter of the holder part 111 is preferably two times to three times or more the external diameter of the body 110.

### Configuration of Stimulation Circuit

Set forth next, with reference to FIG. 5, is a more detailed description of the circuit configuration of the stimulation circuit 205 and the coil portion 212 included in the circuit block 103.

FIG. 5 is a functional block diagram showing the stimulation circuit and the coil portion related to the first embodiment of the invention. The stimulation circuit 205 includes a communication section 302, a stimulation parameter setting section 304, an electrode configuration setting section 305, an oscillation section 306, and a control section 303. The stimulation circuit 205 also includes a charging section 308, a rechargeable battery 309, and a switch section 307.

The rechargeable battery 309 is, for example, a rechargeable battery, such as a lithium-ion battery. The rechargeable battery 309 supplies reserved electric power to respective blocks that constitute the stimulation circuit 205.

The coil portion 212 is, for example, a resonant circuit constituted by a coil and a capacitor. The coil portion 212 receives electromagnetic waves for charging that are transmitted from a controller outside a living body, when charging the rechargeable battery 309. An alternating current that is generated from the coil portion 212 with this reception is output to the charging section 308. Additionally, the coil portion 212 receives the electromagnetic waves that are transmitted from the controller outside the living body and have predetermined information carried thereon, and the received electromagnetic waves are output to the communication section 302 from the coil portion 212 concerned.

The charging section 308 has a rectifier circuit built therein, and converts the alternating current output from the coil portion 212 into a direct current to acquire electric power. The rechargeable battery 309 is charged with the acquired electric power.

The communication section 302 demodulates the electromagnetic waves received by the coil portion 212, and retrieves the information carried on the electromagnetic waves. Then, the retrieved information is output to the stimulation parameter setting section 304 and the electrode configuration setting section 305 via the control section 303. The information output to the stimulation parameter setting section 304 is information (hereinafter referred to as a “stimulation parameter”) on the stimulation intensity of an electric stimulation signal, and the information output to the electrode configuration setting section 305 is inthrmation (hereinafter referred to as “electrode configuration information”) on the electrode configuration. Since the stimulation intensity of the electric stimulation signal is determined depending on the pulse voltage, pulse current, pulse width, or the frequency of the electric stimulation signal, the stimulation parameter is a signal indicating the value of the pulse voltage or the like. Additionally, the electrode configuration information is a signal including information for changing the polarity of an electric stimulation signal, and information for making the switch section 307 select a stimulation electrode 105 that outputs the electric stimulation signal.

The stimulation parameter setting section 304 generates a stimulation intensity change signal for changing the stimulation intensity of the electric stimulation signal generated in the oscillation section 306, on the basis of the stimulation parameter input from the communication section 302.

The electrode configuration setting section 305 generates an electrode configuration selection signal for selecting the stimulation electrode 105 that outputs the electric stimulation signal generated in the oscillation section 306, on the basis of the electrode configuration information input from the communication section 302. In addition, the stimulation intensity change signal output from the stimulation parameter setting section 304 is output to the oscillation section 306, and the electrode configuration selection signal output from the electrode configuration setting section 305 is output to the switch section 307.

The oscillation section 306 generates an electric stimulation signal and outputs the signal to the switch section 307, on the basis of the stimulation intensity change signal input from the stimulation parameter setting section 304.

The switch section 307 determines a stimulation electrode 105 that outputs the electric stimulation signal input from the oscillation section 306, on the basis of the electrode configuration selection signal input from the electrode configuration setting section 305. In addition, the control section 303 is, for example, a microcomputer or the like, and controls the respective blocks of the stimulation circuit 205.

### Implantation Procedure of Electric Stimulator

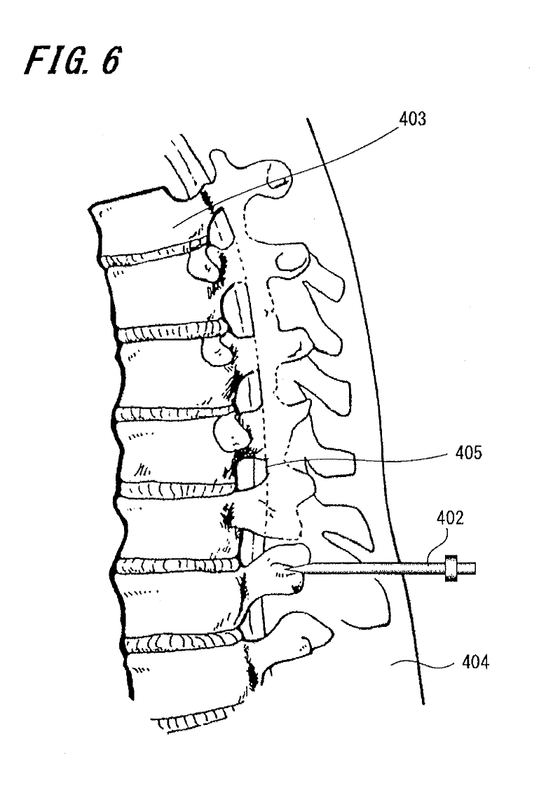

An example of a procedure involving implanting the electric stimulator 101 into an epidural space, and performing an electric stimulation of the nerves of the spinal cord with the electric stimulator 101, will be described with reference to FIGS. 6 to 10.

First, a doctor determines a target stimulation site of the spinal cord in advance, on the basis of the pattern of pain distribution in a patient. Then, an insertion is carried out from a patient's back side under X-ray illumination, and tubular insertion tool or tubular lead-in tool 42 is inserted into an epidural space 405. In the illustrated embodiment disclosed by way of example, the tubular insertion/lead-in tool is an epidural needle 402. As a position where the epidural needle 402 is inserted into the epidural space 405, generally, a position that is lower than a target stimulation site by three vertebral levels or more is selected (refer to FIG. 6).

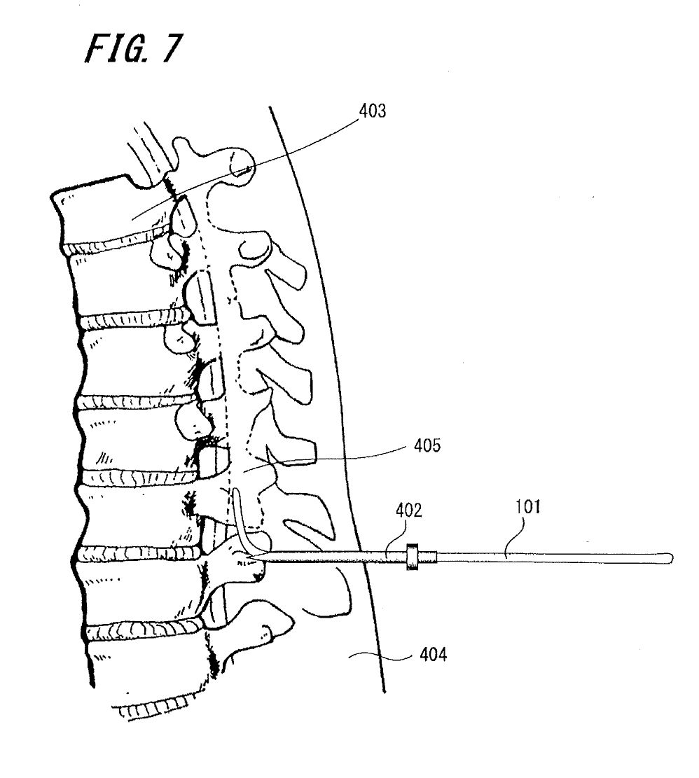

Next, the doctor passes the distal end 114 (refer to FIG. 1) of the electric stimulator 101 through the epidural needle 402, and inserts the electric stimulator 101 into a living body 404. Then, as shown in FIG. 7 (the holder part 111 of the electric stimulator 101 is not shown), the electric stimulator 101 is inserted into the epidural space 405 through the epidural needle 402 by pushing the holder part 111 in the axial direction.

Subsequently, the doctor pushes the holder part 111 further in the axial direction, moves the electric stimulator 101 upward in the epidural space 405, and locates the stimulation electrodes 105 of the electric stimulator 101 near the stimulation site.

Next, the doctor operates the controller outside of the body to perform nerve stimulation, moving the position of the stimulation electrodes 105 little by little. At this time, in the stimulation circuit 205 of the electric stimulator 101, an electric stimulation signal with predetermined intensity is generated on the basis of the doctor's operation, the generated electric stimulation signal is output to the stimulation electrodes 105, and nerve stimulation of a portion near the position of the stimulation electrodes 105 is performed. Then, the doctor determines an optimal position of the stimulation electrodes 105, hearing the reaction to the nerve stimulation of the patient.

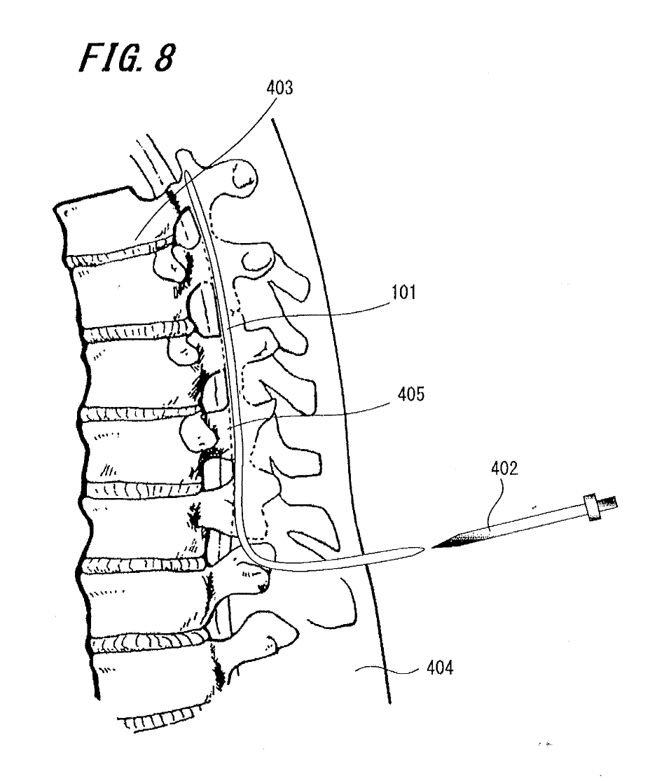

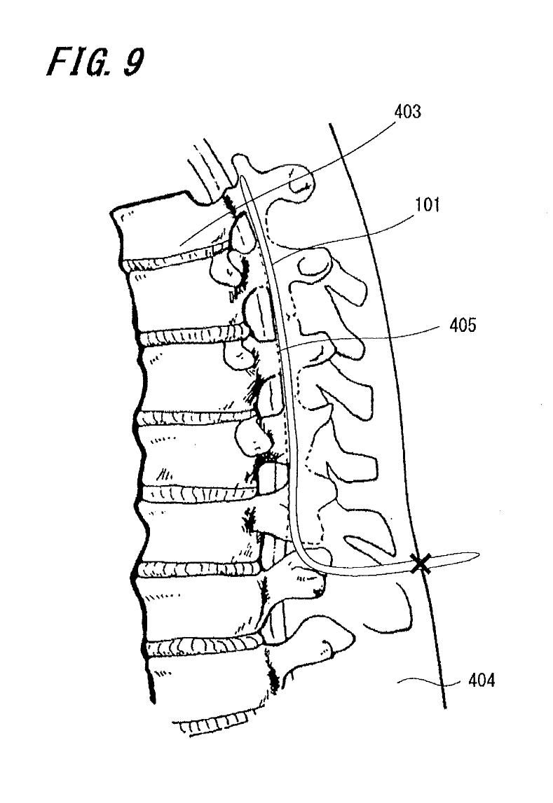

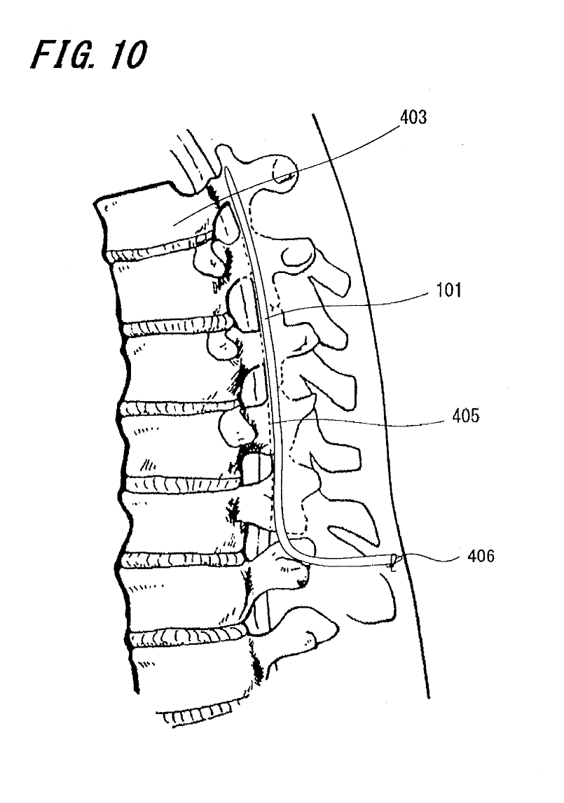

Subsequently, the doctor removes the epidural needle 402 from the living body 404 after cutting the holder part 111 of the electric stimulator 101 so that the electric stimulator 101 is wholly implanted in the living body 404 (refer to FIG. 8). Then, finally, after the portion (equivalent to a portion of the support 104) of the electric stimulator 101 that sticks out from the living body 404 is cut (refer to FIG. 9), the cut portion and the living body 404 are sewn with thread 406 so that the cut electric stimulator 101 is fixed in the state of being wholly implanted in the living body 404 as generally shown in FIG. 10. This treatment is performed so as not to develop an infection or the like from an insertion opening of the electric stimulator 101.

It is also possible insert the electric stimulator 101 into the epidural space 405 through a cannula instead of the epidural needle 402. After the cannula is led to near a location where stimulation is performed through the epidural needle in advance and the epidural needle is removed, an electric stimulator is passed through this cannula and inserted into a living body. Here, the cannula refers to a tube that is made of resin material(s) that have pliability and bio compatibility, for example, materials, such as silicone or polyurethane.

As described above, in the first embodiment of the invention, the electrode block, the circuit block, and a portion of the support are formed in a shape such that these can be inserted through the inside of the epidural needle. Therefore, there are effects that a doctor can operate the support to move the stimulation electrodes to a predetermined position via the epidural needle, and the stimulation electrodes are stably placed at the position for a prolonged period of time. A doctor can also operate the support to remove the electric stimulator relatively easily out of the body.

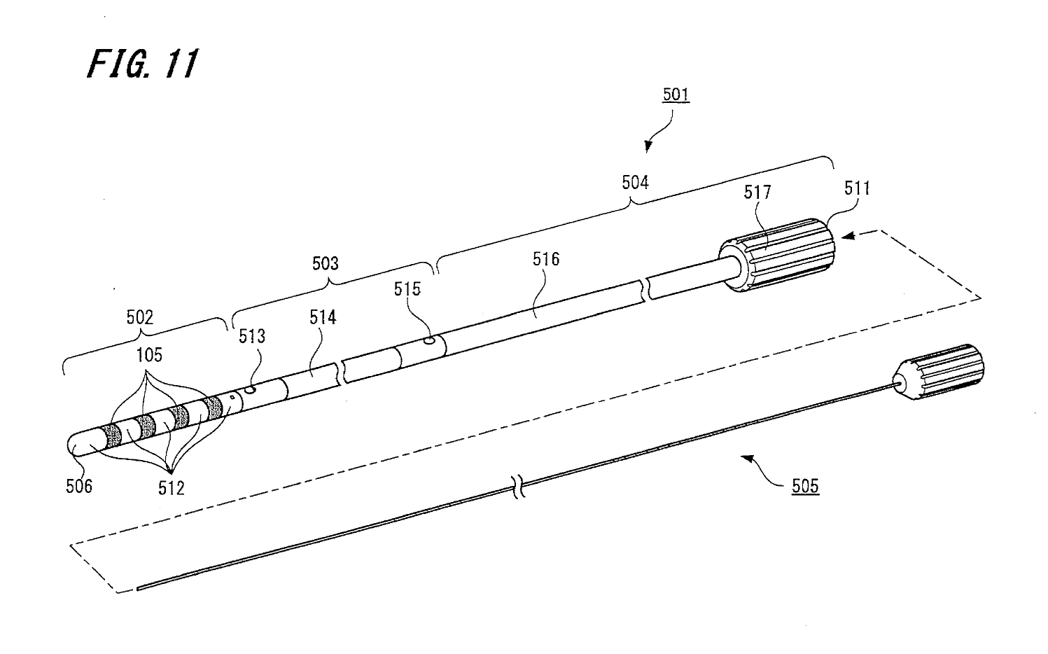

A second embodiment of the electric stimulator will now be described with reference to FIGS. 11 to 14. An electric stimulator 501 according to this second embodiment shown in FIGS. 11 to 14 includes a cylindrical hole (hereinafter referred to as a “lumen for a stylet”) for inserting a stylet 505 into the electric stimulator 101. Features of this second embodiment of the electric stimulator that are similar to those described above are identified by similar reference numerals, and a detailed description of such features may not be repeated.

### Configuration of Electric Stimulator

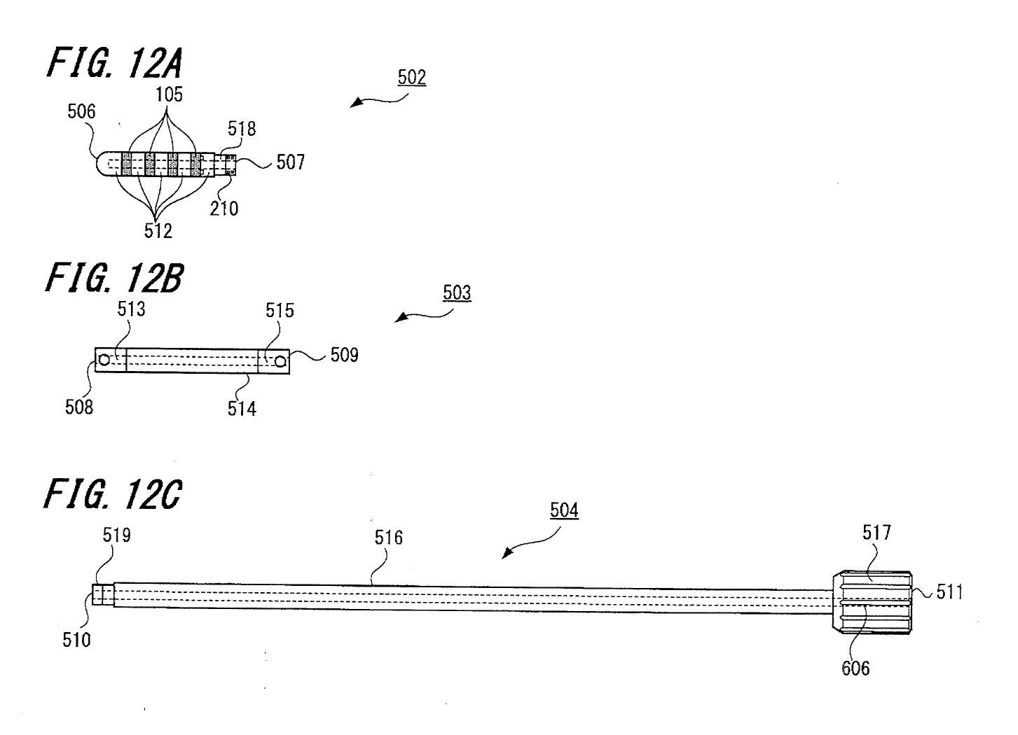

The general configuration of the electric stimulator according to the second embodiment will be described with reference to FIG. 11 and FIGS. 12A to 12C. Similar to the electric stimulator 101 described above, the electric stimulator 501 is elongated, formed in the shape of a rod (rod-shaped), generates an electric stimulation signal, and stimulates nerves or the like in a living body by the stimulation signal. When the electric stimulator 501 is used to stimulate the nerves of the spinal cord, the electric stimulator 501 is implanted in the living body (for example, an epidural space where the distance between the spinal dura mater and the ligamentum flavum is about 5 mm). Therefore, as for the electric stimulator 501, it is preferable that the diameter from a distal end 506 to a predetermined portion of a support 504 (that will be described below) is about 1 mm to 3 mm.

The electric stimulator 501 is mainly comprised of an electrode block 502, a circuit block 503, and the support 504. In addition, the electrode block 502 and the circuit block 503 are attachable and detachable by a connector part 513, and the circuit block 503 and the support 504 are attachable and detachable by a connector part 515. More specifically, as shown in FIGS. 12A to 12C, as for the electrode block 502 and the circuit block 503, a connector part 518 on the electrode block 502 side and the connector part 513 on the circuit block 503 side are fixed together, for example, with a screw or the like. Similarly, as for the circuit block 503 and the support 504, the connector part 515 on the circuit block 503 side and a connector part 519 on the support 504 side are fixed together with a screw or the like. When the electrode block 502, the circuit block 503, and the support 504 are connected together, these respective blocks have the lumen for a stylet that communicates with the axial direction of the blocks. The lumen for a stylet opens to a proximal end 511, and extends up to the vicinity of the distal end 506. In addition, the diameter of the lumen for a stylet is preferably approximately equal to or slightly larger than the diameter of the stylet 505.

The distal end 506 of the electrode block 502 possesses a substantially hemispherical shape, and the other portions of the electrode block 502 possess a substantially cylindrical shape. The radius of the substantially hemispherical portion of the distal end 506 is preferably about 0.5 mm to 1.5 mm, and the outer diameter of the other substantially cylindrical portion is preferably about 1 mm to 3 mm.

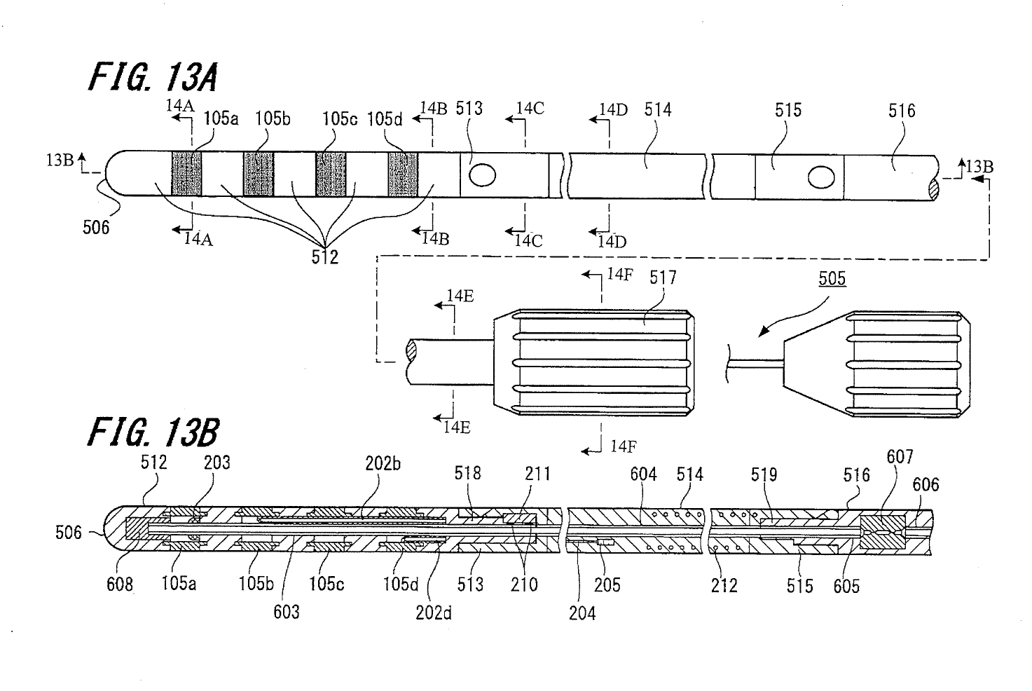

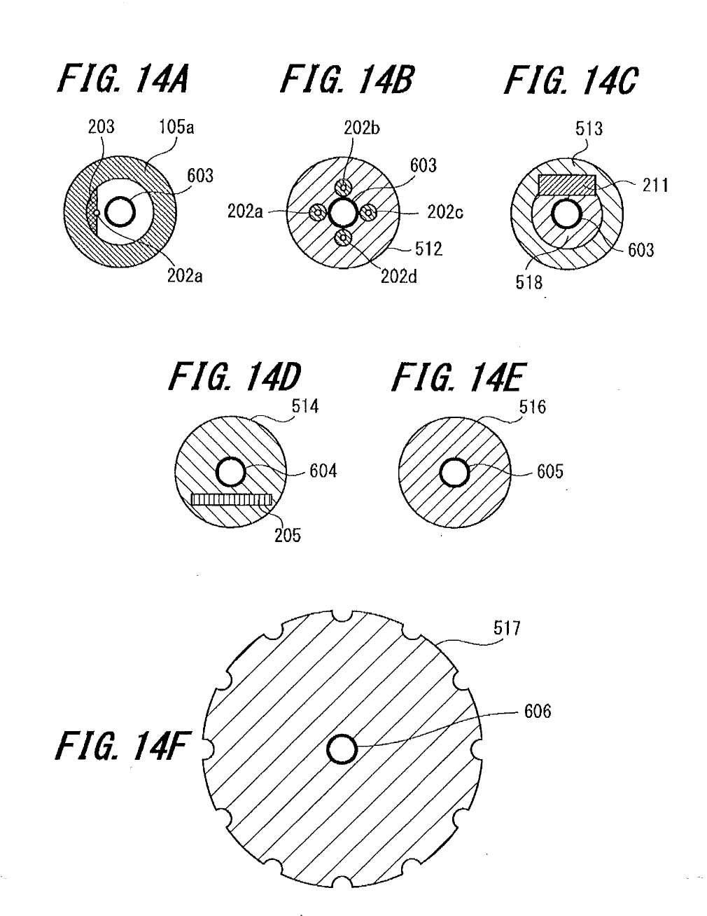

The electrode block 502 includes the four stimulation electrodes 105 for stimulating nerves or the like, and a body that includes exposed body portions 512 arranged at equal intervals such that, when the electric stimulator 501 is arranged in a living body, the respective stimulation electrodes 105 are exposed to the living body. The connector part 518 connects a proximal end 507 of the body 512 and a distal end 508 of the circuit block 503 so that the two blocks are continuous. The internal configuration of the electrode block 502 will be described below with reference to FIGS. 13A and 13B and FIGS. 14A to 14F.

The circuit block 503 possesses a substantially cylindrical shape with the same outer diameter as the outer diameter of the electrode block 502. The circuit block 503 includes the connector part 513 that is connected to the connector part 518 of the electrode block 502 such that the distal end 508 is continuous with the proximal end 507 of the body 512 of the electrode block 502. Additionally, the circuit block 503 is provided with a body 514 that is continuous with the connector part 513. Moreover, the circuit block 503 includes the connector part 515 that is continuous with a proximal end 509 side of the body 514 and connects the proximal end 509 and the support 504. The internal configuration or the like of the circuit block will be described below with reference to FIGS. 13A and 13B and FIGS. 14A to 14F.

The support 504 includes the connector part 519 connected to the circuit block 503, a body 516 possessing a substantially cylindrical shape with the same outer diameter as the outer diameter of the electrode block 502, and a substantially cylindrical holder part 517 that has a larger outer diameter than the as the outer diameter of the body 516.

The connector part 519 of the support 504 is connected to the connector part 515 of the circuit block 503 such that a distal end 510 of the body 516 is continuous with the circuit block 503. The body 516 is a portion that connects the connector part 519 and the holder part 517 arranged on the proximal end 511. In addition, the holder part 517 is a place that is held when a doctor inserts the electric stimulator 501 into a living body. The internal configuration or the like of the support 504 will be described below with reference to FIGS. 13A and 13B and FIGS. 14A to 14F.

The internal configuration of the electric stimulator 501 according to the second embodiment is now described with reference to FIGS. 13A, 13B and FIGS. 14A to 14F. The internal configuration of the electrode block 502 will first be described.

A pipe 603 is made of materials that have biocompatibility, insulation, and pliability, for example, PTFE or ETFE, and is formed in a hollow, substantially cylindrical shape. It is desirable that the external diameter of the pipe 603 be about 0.1 to 1 mm, and the internal diameter of the pipe 603 be approximately equal to or slightly longer than the diameter of the stylet 505 so that the stylet 505 can pass through the inside of the pipe 603. One end (the end on the distal end 506) of such a pipe 603 is connected to a receiving portion 608.

The receiving portion 608 is made of stainless steel and possesses a substantially cylindrical shape, and a substantially cylindrical hole (blind hole) opens to the axial center. The total axial length and diameter of this hole are respectively rather shorter, compared to the total axial length and external diameter of the receiving portion 608. Additionally, the inner diameter of the hole of the receiving portion 608 is preferably approximately equal to the external diameter of the pipe 603 so that the pipe 603 can be fixed so as not to move perpendicular to the axial direction or the axis. The pipe 603 and the receiving portion 608 arc housed in and fixed to an outer layer part (surrounding part) made up of the body 512, the stimulation electrodes 105, and the connector part 518.

The body 512 is made of resin materials that have pliability and biocompatibility, for example, materials, such as silicone or polyurethane. The distal end 506 of the body 512 is substantially hemispherical as described above, and the radius of the distal end is preferably within a range of about 0.5 mm to 1.5 mm. Portions other than the distal end 506 of the body 512 possess a hollow, substantially cylindrical shape.

The internal diameter of the portion possessing the hollow, substantially cylindrical shape differs in the portion of the body 512 that contacts the receiving portion 608 and the portion of the body 512 that contacts the pipe 603. The internal diameter of the portion that contacts the receiving portion 608 is approximately equal to the external diameter of the receiving portion 608 so as to fix the receiving portion 608. Additionally, the internal diameter of the portion that contacts the pipe 603 is approximately equal to the external diameter of the pipe 603 so as to fix the pipe 603. The four stimulation electrodes 105 are fixed to a portion of the body 512 that possesses a hollow, substantially cylindrical shape so that the stimulation electrodes 105 are exposed at the outer surface of the body 512, as described above. The stimulation electrodes 105 are the same as the stimulation electrodes 105 included in the electric stimulator 101 of the first embodiment, and so a detailed description of such features is omitted.

One end (the distal end 506) of each of the conducting wires 202a to 202d is bonded to a respective one of the stimulation electrodes 105a to 105d (refer to FIG. 14A) with solder 203, and the other end (the proximal end 507) of each of the conducting wires 202a to 202d is electrically connected to the connector part 518. In addition, locations other than the locations of the conducting wires 202 that are bonded with the solder 203 and the locations of the conducting wires 202 that are electrically connected to the connector part 518 are completely embedded in the body 512 (refer to FIG. 14B).

The connector part 518 is formed of the same material as the body 512, and is formed as a cutout part or reduced outer diameter part having an outer diameter different from the external diameter of the substantially cylindrical body 512. This cutout part extends a predetermined distance in the axial direction from the proximal end 507 (refer to FIG. 12A). In addition, a contact surface between this cutout part and the electrical connecting portion 211 is planar, the four connector pins 210 (refer to FIG. 12A) are arranged on the planar surface of the cutout part so as to be exposed, and the conducting wires 202a to 202d are electrically connected to the four connector pins 210, respectively.

Next, the internal configuration of the circuit block 503 (refer to FIG. 12B) is described.

A pipe 604 is arranged in the circuit block 503 and is the same as the pipe 603 except for differences in length. The pipe 604 is housed in and fixed to an outer layer part including the connector part 513, the body 514 that is continuous with the connector part 513, and the connector part 515 that is continuous with the body 514 (refer to FIG. 13B).

The connector part 513 is made of the same material (polyurethane or silicone) as the body 512 except for the electrical connecting portion 211. A hole of almost the same shape and size as the external diameter of the connector part 518 is provided in this connector part 513 and opens in the axial direction in this connector part 513 so that the connector part 513 can be connected to the connector part 518 of the electrode block 502 (refer to FIG. 14C). The external diameter of this connector part 513 is approximately equal to the external diameter of the body 512. Additionally, the connector part 513 includes the electrical connecting portion 211 that is independently and electrically connected to the four connector pins 210, respectively, when this connector part 513 is connected to the connector part 518 of the electrode block 502. The connector part 515 provided on the proximal end 509 of the circuit block 503 has the same shape and size as the connector part 513 provided on the distal end 508, though does not include a portion like the electrical connecting portion 211.

The body 514 is continuous with the connector parts 513 and 515 and is made of the same material as the connector parts 513 and 514. The body 514 possesses a hollow, substantially cylindrical shape, and is formed such that the external diameter of the body 514 is approximately equal to the external diameter of the connector parts 513 and 515, and the internal diameter of the body 514 is approximately equal to the external diameter of the pipe 604. In addition, similar to the construction described in FIG. 3B, the stimulation circuit 205 and the coil portion 212 are embedded in the body 514.

The stimulation circuit 205 is connected to the electrical connecting portion 211 via the conducting wires 204 embedded in the body 514 such that the generated electric stimulation signal is independently supplied to the respective stimulation electrodes 105a, 105b, 105c, 105d. The electric configuration of the stimulation circuit 205 and the coil portion 212 is the same as described above in connection with the illustration in FIG. 5 and so a detailed description of the stimulation circuit 205 and the coil portion 212 is not repeated.

Next, the internal configuration of the support 504 shown generally in FIG. 12C will be described.

As shown in FIG. 13B, pipes 605 and 606 arranged in the support 504 are the same as the pipes 603 and 604,except for the length. A valve 607 is provided between the pipe 605 and the pipe 606 in the axial direction.

The valve 607 is made of an elastic material (a soft material is preferable) with biocompatibility, like silicone rubber for example. The valve 607 has a first slit that opens to one axial end face at the pipe 605 and does not open to the other axial end face, and a second slit that opens to the other axial end face at the pipe 606 and does not open to the one axial end face, and the other axial end face of the first slit connects to the one axial end face of the second slit. The valve 607 helps prevent liquids, such as body fluids, from entering the insides of the electrode block 502 and the circuit block 503 from the pipe 606, even if the stylet 505 is inserted and removed via the valve 607.

The pipes 605 and 606 and the valve 607 are housed in and fixed to an outer layer part made up of the connector part 519, the body 516 that is continuous with the connector part 519, and the holder part 517 that is provided on the proximal end 511 of the body 516.

The connector part 519 is made of, for example, polyurethane or silicone and is formed in the same shape and size as the connector part 518 of the electrode block 502 described above (refer to FIG. 12C). It is noted that means for performing electric connection with the outside, such as the connector pins 210, may not be provided, thus differing from the connector part 518 in that regard.

The body 516 is made of the same material as the connector part 519, and possesses a hollow, substantially cylindrical shape. The external diameter of the body 516 is approximately equal to the external diameter of the body 514 of the circuit block 503.

The holder part 517 is made of materials, such as a plastic, and possesses a hollow, substantially cylindrical shape. The internal diameter of the holder part 517 is approximately equal to the external diameter of the respective pipes 603 to 606. Additionally, since the holder part 517 is a part that is held when the electric stimulator 501 is inserted into the inside of a body, the external diameter of the holder part 517 is preferably two times to three times or more the external diameter of the body 516.

As described above, the lumen for a stylet is formed by the receiving portion 608, the pipes 603 to 606, the valve 607, and the holder part 517.

The procedure of implanting the electric stimulator 501 when an electric stimulation of the nerves of the spinal cord is performed by the electric stimulator 501 will now be described.

After the epidural needle 402 is inserted up to the epidural space 405 (refer to FIG. 6) of the body, the stylet 505 is inserted into the stylet lumen of the electric stimulator 501 from the proximal end 511 of the holder part 517 of the electric stimulator 501. The distal end 506 (refer to FIG. 11) of the electric stimulator 501 is passed through the epidural needle 402, and the receiving portion 608 of the electric stimulator 501 is pushed by the stylet 505 to move the stimulation electrodes 105 to a targeted stimulation site of the epidural space 405. Then, after the controller located outside the body is operated to determine an optimal position of the stimulation electrodes 105, the stylet 505 is extracted from the electric stimulator 501. The rest of the procedure is the same as the procedure described above with reference to FIGS. 8 to 10, and so a detailed description of the procedure is not repeated.

As described above, because the second embodiment includes the lumen for a stylet, a stylet can be used when the electric stimulator is implanted in a living body. Therefore, there are effects that implantation of the electric stimulator into the body can be performed relatively easily, and the precision of the arrangement of the stimulation electrodes into the living body can be improved. Other benefits such as those discussed above with respect to the first embodiment are also realized with this second embodiment.

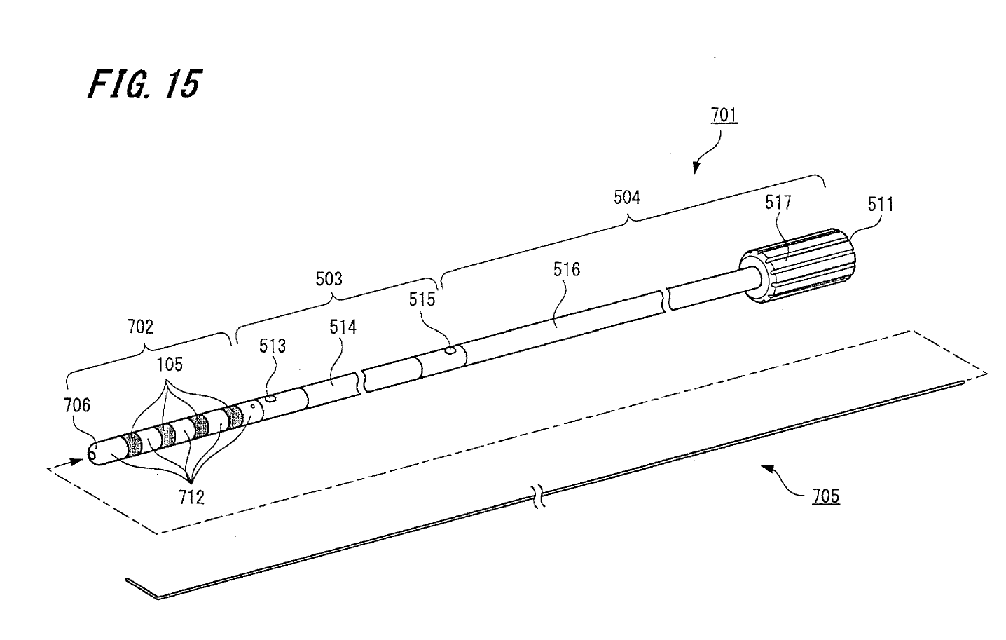

A third embodiment of the electric stimulator is illustrated in FIGS. 15 to 17. An electric stimulator 701 according to the third embodiment shown in FIGS. 15 to 17 includes a cylindrical hole (hereinafter referred to as a “lumen for guide wire”) for inserting a guide wire into the electric stimulator 101 related to the first embodiment. Features of this third embodiment of the electric stimulator that are similar to those described above are identified by similar reference numerals, and a detailed description of such features may not be repeated.

### Configuration of Electric Stimulator

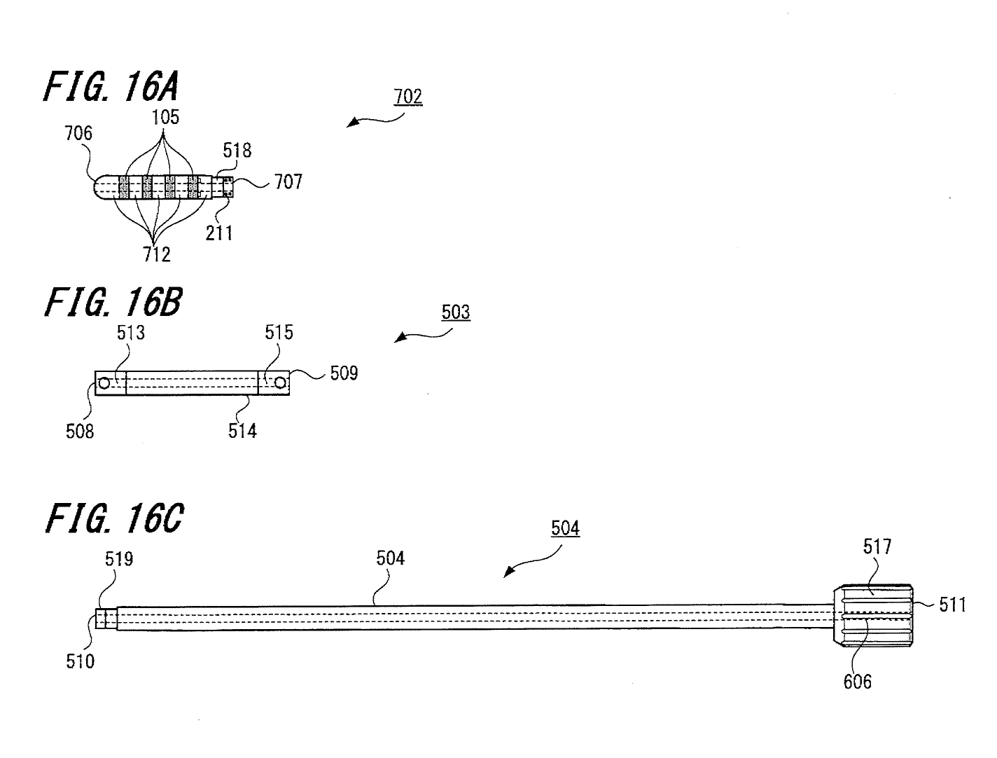

First, the general configuration of the electric stimulator according to the third embodiment will be described with reference to FIG. 15 and FIGS. 16A to 16C.

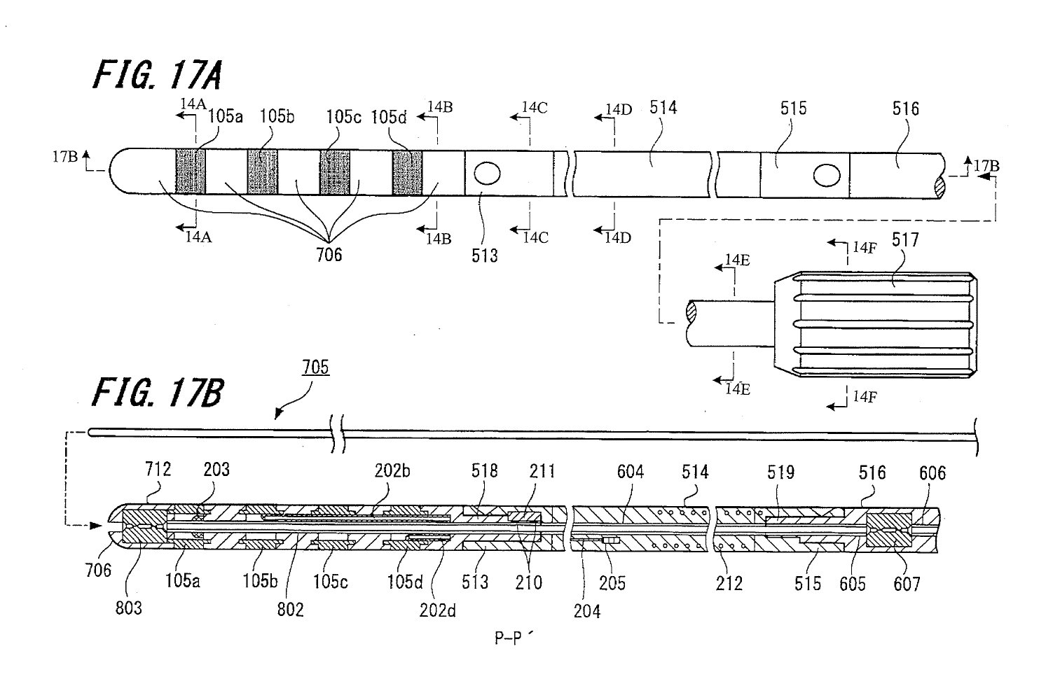

Similar to the electric stimulators 101 and 501 described above, the electric stimulator 701 according to the third embodiment is formed in the shape of a rod (i.e., is rod-shaped), generates an electric stimulation signal, and stimulates nerves or the like in a living body by the stimulation signal. The electric stimulator 701 has the axially extending lumen for a guide wire as described above, and the electric stimulator 701 is hollow and possesses a substantially cylindrical shape. The lumen for a guide wire is provided as a through hole that opens to the proximal end 511 and also opens to the distal end 706. Therefore, the electric stimulator 701 has a configuration in which the electrode block 502 of the electric stimulator 501 of the second embodiment is replaced with a substantially cylindrical electrode block 702 formed with a hollow portion that communicates in the axial direction. The internal configuration of the electrode block 702 will be described in FIGS. 17A and 17B.

The internal configuration of the electrode block 702 in the electric stimulator according to the third embodiment is described next with reference to FIGS. 17A and 17B and the above-described FIGS. 14A to 14F.

Although FIGS. 14A to 14F, as described above, are cross-sectional views showing the internal structure of a predetermined location in a radial direction with respect to the axis of the electric stimulator 501 related to the second embodiment of the invention, these drawings are also cross-sectional views showing the internal structure in a radial direction with respect to the electric stimulator 701 according to the third embodiment.

Here, only the electrode block 702 will be described (refer to FIG. 16A).

The electric stimulator 701 includes a pipe 802 that is the same as the pipes 604 to 606, except for the axial length of the pipe.

The electric stimulator 701 also includes a valve 803 that is the same as the valve 607. The valve 803 has a first slit that opens to one axial end face at the pipe 802 and does not open to the other axial end face, and a second slit that opens to the other axial end face on the distal end 706 side of the body 712, and does not open to the one axial end face, and the other axial end face of the first slit internally crosses the one axial end face of the second slit. That is, one slit opens to one axial end face while the other slit opens to the other axial end face. Even if a guide wire 705 is inserted and removed via the valve 803, liquids, such as body fluids, can be prevented from entering the insides of the electrode block 702 and the circuit block 503 from the hole provided in the distal end 706 of the body 712.

The body 712 is made of, for example, materials, such as silicone or polyurethane. The body 712 has a substantially cylindrical hole in the distal end 706 thereof. The inner diameter of this hole is approximately equal to the external diameter of the pipe 802. In addition, the external diameter of the body 712 is the same as that of the body 512 (refer to FIGS. 13A and 13B) of the second embodiment. The connector part 518 is the same as that described in the second embodiment, and so a detailed description of the connector part 518 is omitted.

The procedure of implanting the electric stimulator 701 when an electric stimulation of the nerves of the spinal cord is performed by the electric stimulator 701 will be described.

First, the epidural needle 402 is inserted up to the epidural space 405 (refer to FIG. 6) in a body, and the guide wire 705 is inserted into the epidural space 405 through the epidural needle 402. Then, a distal end of the guide wire 705 is advanced up to a targeted stimulation site. Subsequently, a proximal end of the guide wire 705 is inserted into the distal end 706 of the electric stimulator 701, the holder part 517 is pushed so as to move the electric stimulator 701 along the guide wire 705, and the stimulation electrodes 105 of the electric stimulator 701 is moved to a targeted stimulation site. Then, after the controller outside the body is operated to determine an optimal position of the stimulation electrodes 105, the guide wire 705 is extracted from the electric stimulator 701. The rest of the procedure is the same as the procedure described above relative to FIGS. 8 to 10, and so a detailed description of the remainder of the procedure is not repeated.

The guide wire lumen included in the electric stimulator as described above allows a guide wire to be used when the electric stimulator is implanted in a living body. Therefore, implantation of the electric stimulator into the body can be performed relatively easily, and the precision of the arrangement of the stimulation electrodes into the living body can be improved. Other affects similar to those described above with respect to the first embodiment are possible here.

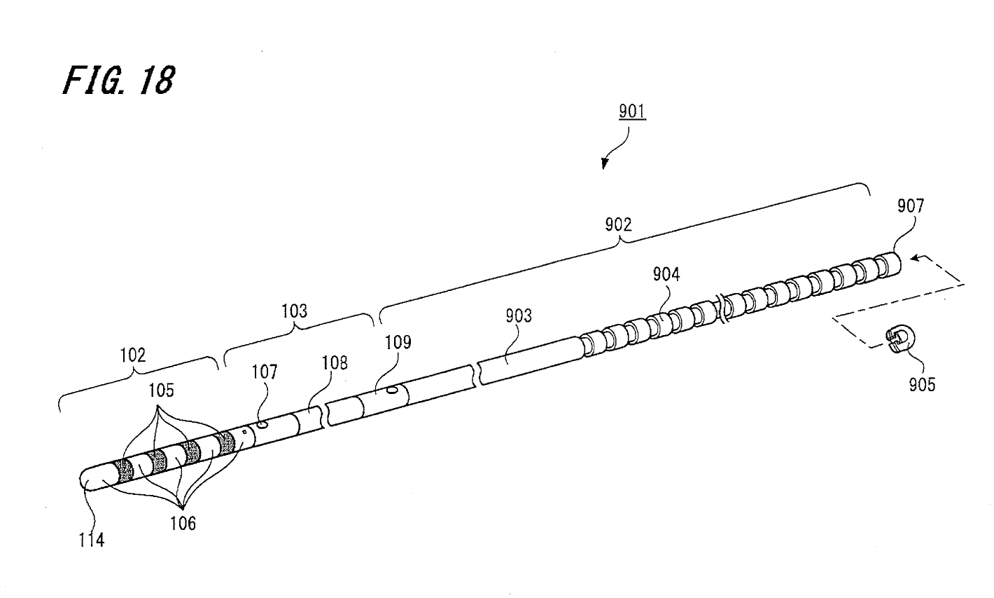

A fourth embodiment of the electric stimulator will be described with reference to FIGS. 18 to 21. The configuration of the electric stimulator 901 according to the fourth embodiment shown in FIGS. 18 to 21 is similar in many respects to the embodiments of the electric stimulators described above. Features and portions of this fourth embodiment of the electric stimulator that are the same as in earlier described embodiments are designated with common reference numerals, and a detailed description of such features will not be repeated. The implantation procedure of the electric stimulator 901 according to this fourth embodiment is generally the same as the implantation procedure of the electric stimulator 101 according to the first embodiment, and so a detailed description of the entire implantation procedure will also not be repeated.

### Configuration of Electric Stimulator

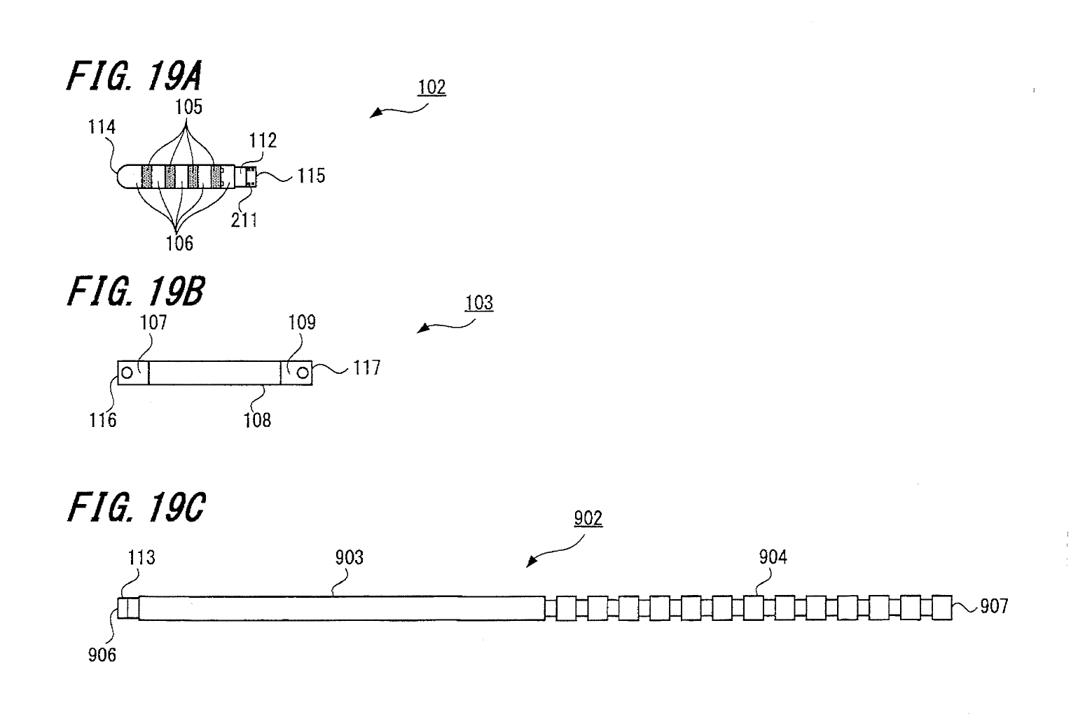

The general configuration of the electric stimulator according to the fourth embodiment will first be described with reference to FIG. 18 and FIGS. 19A to 19C.

FIG. 18 is a perspective view showing an overall electric stimulator related to the fourth embodiment of the invention.

FIGS. 19A to 19C are exploded external views when the electric stimulator shown in FIG. 18 is seen from the top face.

Similarly to the above-described respective electric stimulators, the electric stimulator 901 is formed in the shape of a rod (i.e., is rod-shaped), generates an electric stimulation signal, and stimulates nerves or the like in a living body by the stimulation signal. The electric stimulator 901, as shown in FIG. 18 and FIGS. 19A to 19C, has a support 902 instead of the support 104 in the first embodiment (refer to FIG. 1) of the electric stimulator 101, and further includes a fixture 905.

The support 902 includes the connector part 113 that is connected to the circuit block 103, a first body 903 that possesses a substantially cylindrical shape with the same outer diameter as the outer diameter of the electrode block 102, and a second body 904 that is continuous with the first body 903.

The connector part 113 is connected to the connector part 109 of the circuit block 103 such that a distal end 906 side of the first body 903 is continuous with the circuit block 103. The first body 903 is a portion that connects the connector part 113 and the second body 904 arranged on a proximal end 907. The second body 904 is configured to be cut so that the electric stimulator 901 is completely implanted in a living body. The detachable fixture 905 (refer to FIG. 18) is provided at a cut surface of the second body 904.

The fixture 905 is made of materials with biocompatibility and flexibility, such as silicone, and is formed in a substantially hemispherical shape. A bottom face of the fixture 905 is formed with a substantially cylindrical cavity into which the cut surface of the second body 904 is inserted, and a lateral face of the fixture 905 is formed with a ring-shaped groove. The diameter of the substantially cylindrical cavity is made approximately equal to or slightly larger than the second body 904 so that the cut surface of the second body 904 can be inserted into the cavity of this substantially cylindrical shape. In addition, the fixture 905 is fixed to the second body 904 by a thread that is passed through the groove provided in the lateral face, in a state where the cut surface of the second body 904 is inserted into the cavity of this substantially cylindrical shape. Thereby, inflammation or a feeling of foreign matter accompanying the contact between the cut surface of the second body 904 and a living body can be prevented.

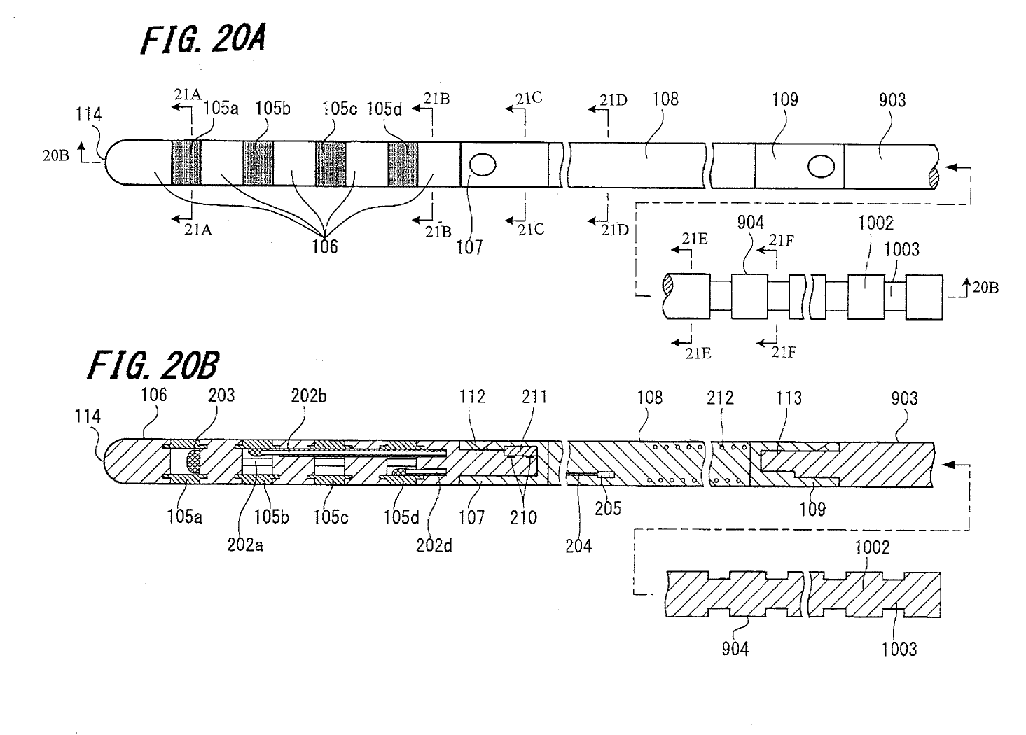

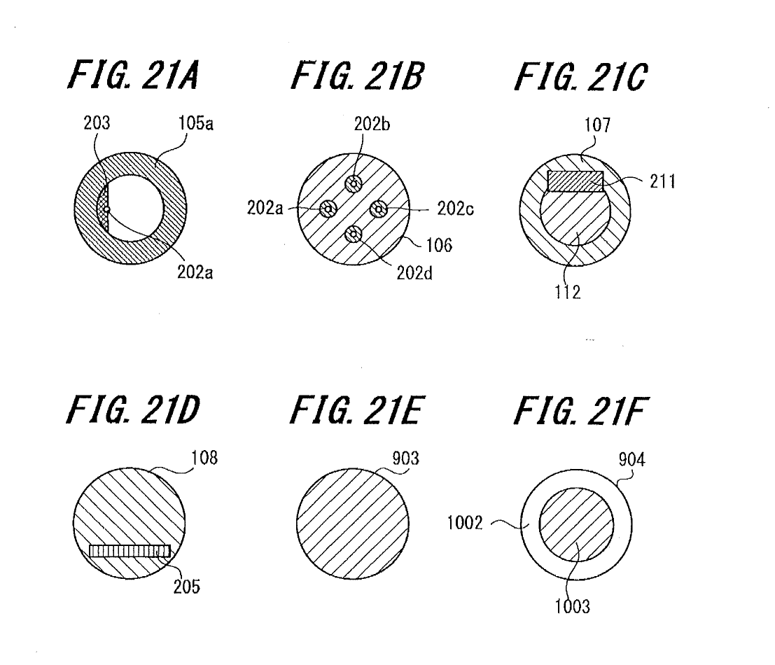

The internal configuration of the support 902 according to the fourth embodiment will next be described with reference to FIGS. 20A and 20B and FIGS. 21A to 21F.

The connector part 113 is made of, for example, polyurethane or silicone, and is the same as the connector part 113 (refer to FIGS. 2C and 3B) of the support 104 described in the first embodiment.

The first body 903 is made of the same material as the connector part 113, and possesses a substantially cylindrical shape. The diameter of the first body 903 is made approximately equal to the external diameter of the body 110 (refer to FIGS. 2C, 3A, and 3B) of the support 104 described in the first embodiment (refer to FIG. 21E).

The second body 904 is continuous with the first body 903, and is made of the same material as the first body 903. The second body 904 is made up of a fixture fixing portion 1002 that is formed in a substantially cylindrical shape with the same outer diameter as the outer diameter of the first body 903, and a relatively easily-cut portion 1003 that is formed with an outer diameter smaller than the outer diameter of the fixture fixing portion 1002 (refer to FIGS. 20B and 21F). The relatively easily-cut portion 1003 refers to a portion that is more easily cut than, for example, the fixture fixing portion 1002. A plurality of the fixture fixing portions 1002 and the easily-cut portions 1003 are alternately provided such that the axes of the two portions 1002, 1003 coincide with each other (i.e., the portions 1002, 1003 are coaxial).

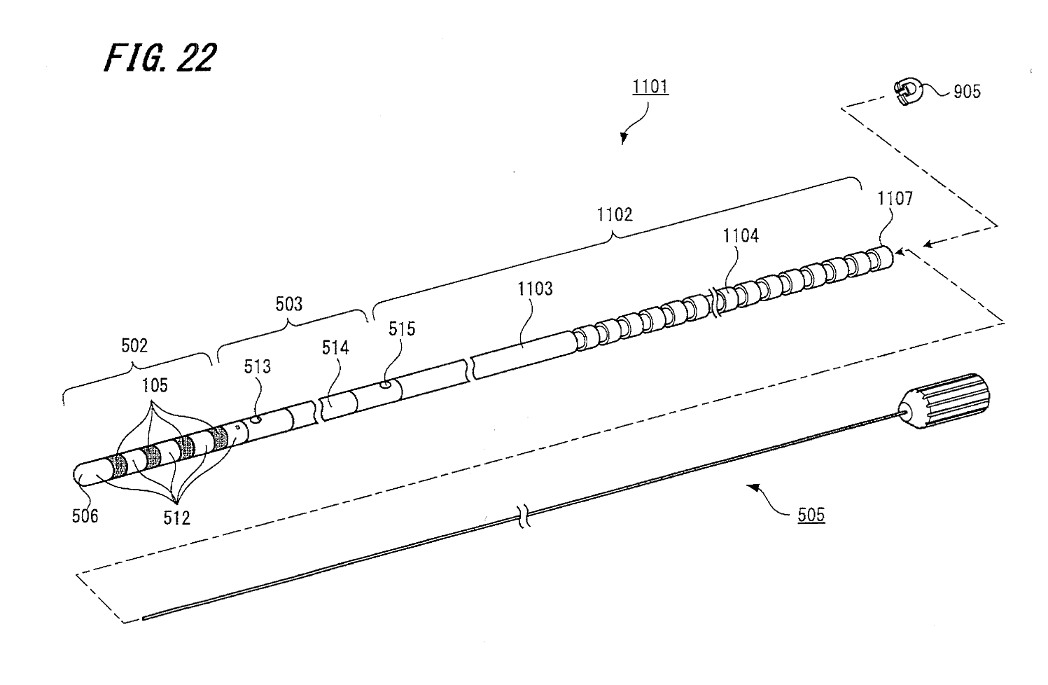

A fifth embodiment of the electric stimulator will be described with reference to FIGS. 22 to 25. The electric stimulator 1101 according to the fifth embodiment shown in FIGS. 22 to 25 is similar to the electric stimulator 901 according to the fourth embodiment, but also includes a lumen for a stylet. The configuration of the electric stimulator 1101 according to the fifth embodiment is similar in other respects to the embodiments of the electric stimulators described above. Features and portions of this fifth embodiment of the electric stimulator that are the same as in earlier described embodiments are designated with common reference numerals, and a detailed description of such features will not be repeated. The implantation procedure of the electric stimulator 1101 according to this fifth embodiment is almost the same as the implantation procedure of the electric stimulator 501 according to the second embodiment, and so a detailed description of the implantation procedure will also not be repeated.

### Configuration of Electric Stimulator

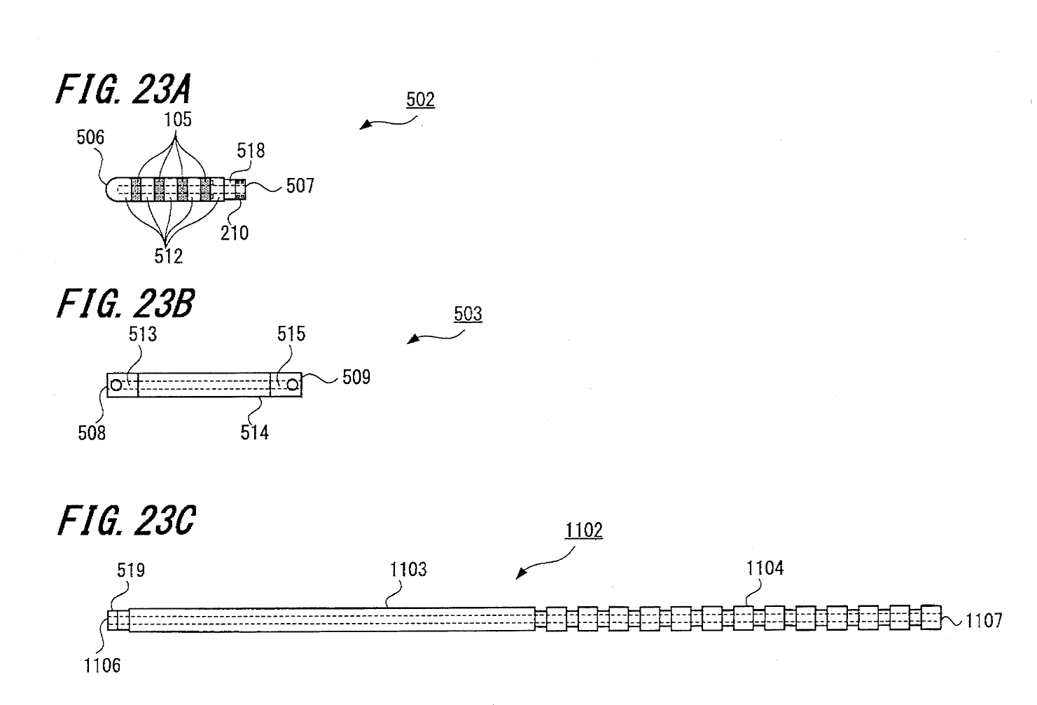

The electric stimulator 1101 is formed in the shape of a rod (i.e., is rod-shaped), generates an electric stimulation signal, and stimulates nerves or the like in a living body by the stimulation signal. The electric stimulator 1101, as shown in FIG. 22 and FIGS. 23A to 23C, has a support 1102 instead of the support 504 (refer to FIG. 11) of the electric stimulator 501 according to the second embodiment, and further includes the above-described fixture 905.

The support 1102 includes the connector part 519 for being connected to the circuit block 503, a first body 1103 that is formed in a substantially cylindrical shape with the same outer diameter as the outer diameter of the electrode block 502, and a second body 1104 that is continuous with the first body 1103.

The connector part 519 of the support 1102 is connected to the connector part 515 of the circuit block 503 such that the distal end 1106 of the first body 1103 is continuous with the circuit block 503. The first body 1103 is a portion that connects the connector part 519 and the second body 1104 arranged on a proximal end 1107 of the first body 1103.

The second body 1104 is configured to be cut so that the electric stimulator 1101 is completely implanted in a living body. The detachable fixture 905 is attached to the cut surface of the cut second body 1104 so as to cover the cut surface. The fixture 905 has already been described above with reference to FIG. 18 and so a detailed description of the fixture will not be repeated. The fixture 905 is fixed to the second body 1104 by a thread. This can prevent inflammation or a feeling of foreign matter accompanying the contact between the cut surface of the second body 1104 and a living body, and can prevent entering of a liquid from a lumen for a stylet on the proximal end 1107 side of the second body 1104.

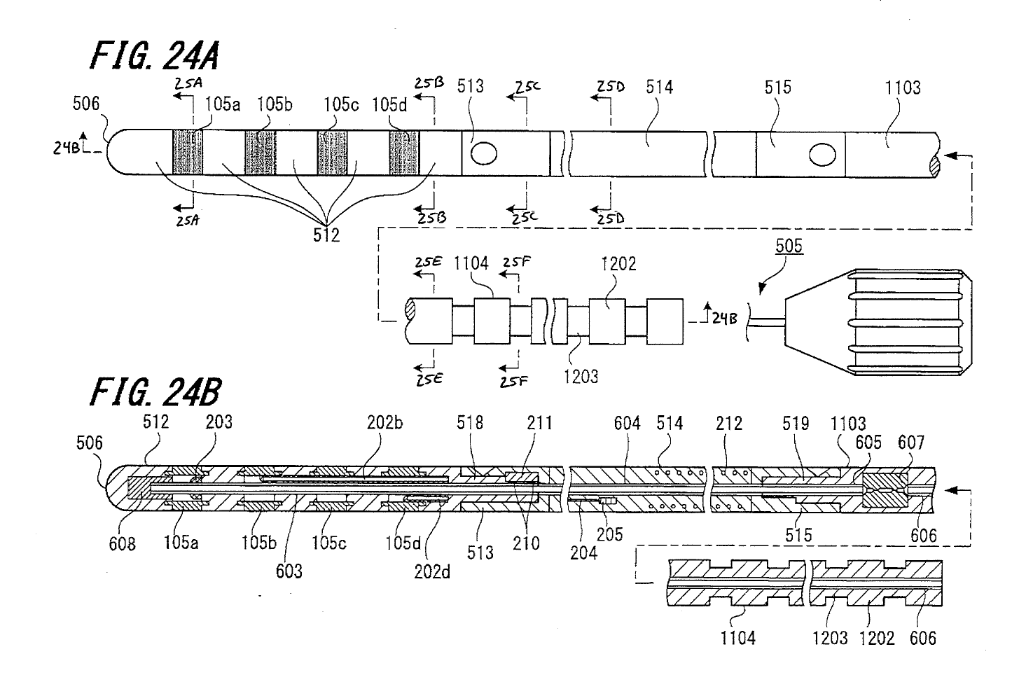

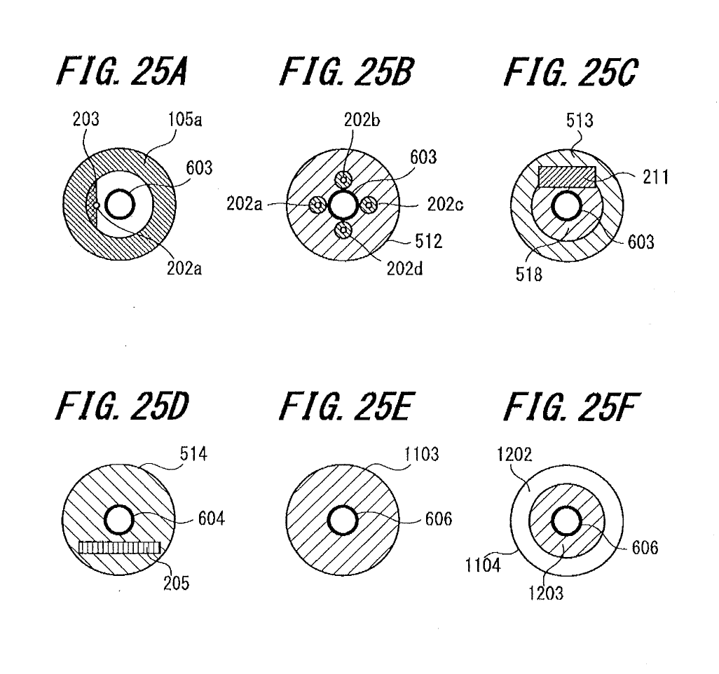

The internal configuration of the support 1102 according to the fifth embodiment will now be described with reference to FIGS. 24A and 24B and FIGS. 25A to 25F.

The support 1102, similar to the second embodiment, includes the pipes 605 and 606, the valve 607 provided between the pipe 605 and the pipe 606 in the axial direction, the connector part 519 in which the pipes 605 and 606 and the valve 607 are housed and fixed, and the first and second bodies 1103 and 1104.

The first body 1103 is made of, for example, polyurethane or silicone and possesses a hollow, substantially cylindrical shape. The external diameter of the first body 1103 is approximately equal to the external diameter of the body 514 of the circuit block 503 (refer to FIG. 25E).

The second body 1104 is continuous with the first body 1103, and is made of the same material as the first body 1103. The second body 1104 is made up of a fixture fixing portion 1202 possessing a substantially cylindrical shape with the same outer diameter as the outer diameter of the first body 1103, and a relatively easily-cut portion 1203 that has an outer diameter smaller than the outer diameter of the fixture fixing portion 1202 (refer to FIG. 25F). The relatively easily-cut portion 1203 refers to a portion that is more easily cut than, for example, the fixture fixing portion 1202. A plurality of the fixture fixing portions 1202 and the relatively easily-cut portions 1203 are alternately provided such that the axes thereof coincide with each other (i.e., they are coaxial). In addition, the connector part 519, the first body 1103, and the second body 1104 have a cavity possessing an inner diameter equal to the external diameter of the pipe 606 in the axial direction on the axis thereof

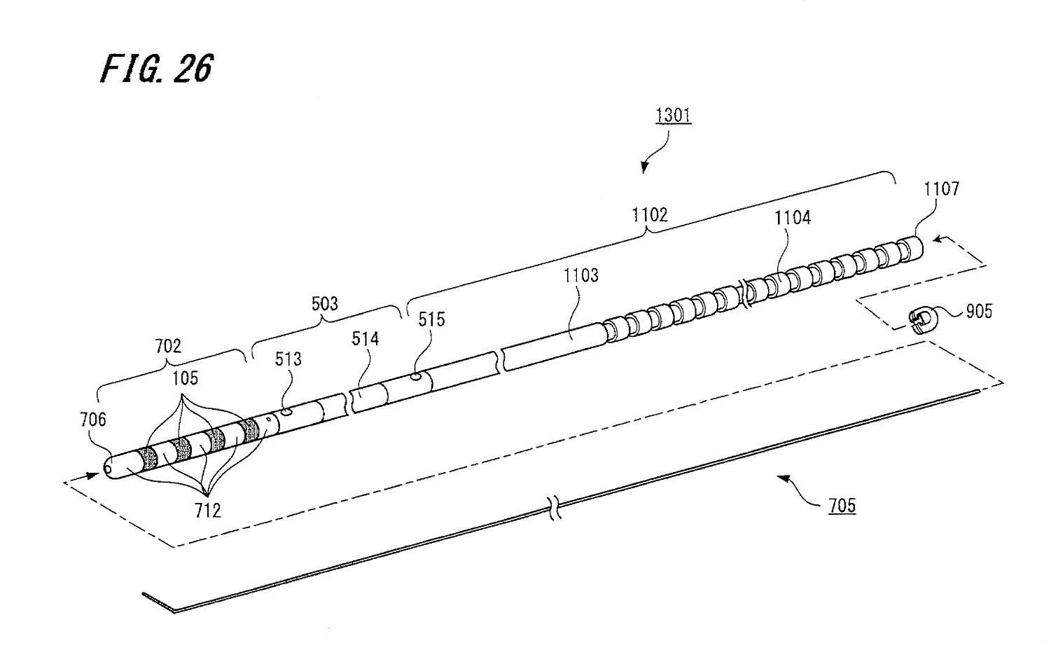

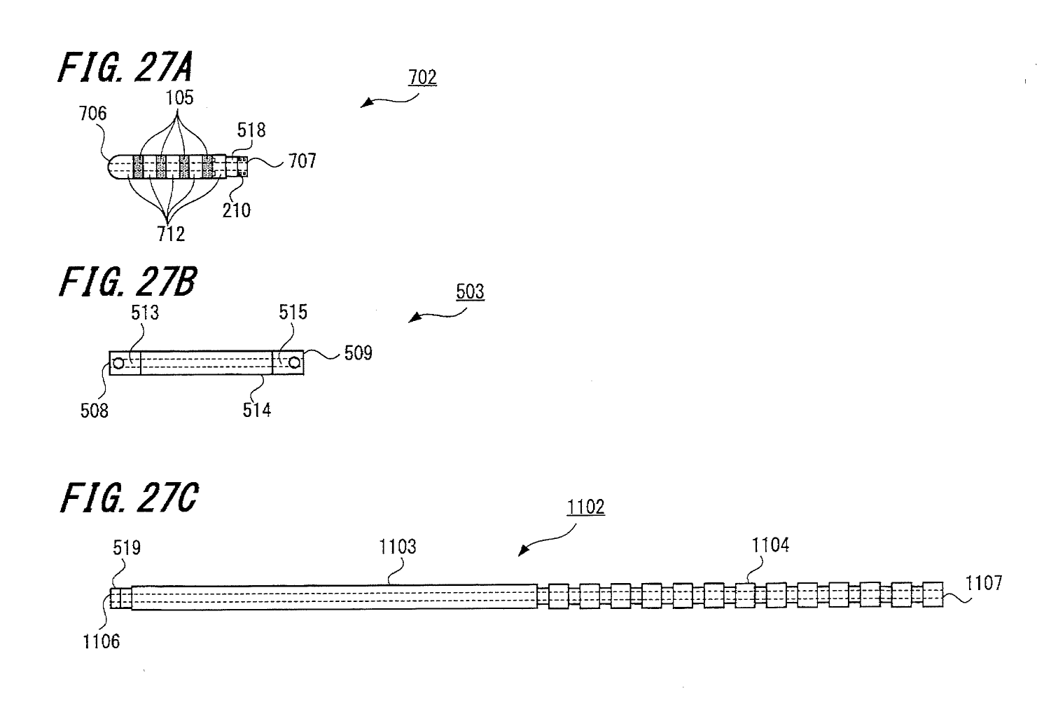

A sixth embodiment of the electric stimulator is illustrated in FIG. 26 and FIGS. 27A to 27C. The electric stimulator 1301 according to the sixth embodiment shown in FIG. 26 and FIGS. 27A to 27C is similar to the electric stimulator 901 according to the fourth embodiment, except the sixth embodiment includes a lumen for a guide wire. Features and portions of this sixth embodiment of the electric stimulator that are the same as in earlier described embodiments are designated with common reference numerals, and a detailed description of such features is not be repeated.

### Configuration of Electric Stimulator

First, the general configuration of the electric stimulator according to the sixth embodiment will be described with reference to FIG. 26 and FIGS. 27A to 27C.

Like the above-described respective electric stimulators, the electric stimulator 1301 according to this sixth embodiment is formed in the shape of a rod (i.e., is rod-shaped), generates an electric stimulation signal, and stimulates nerves or the like in a living body by the stimulation signal. The electric stimulator 1301, as shown in FIG. 26 and FIGS. 27A to 27C, is provided with the electrode block 702 according to the third embodiment, instead of the electrode block 502 (refer to FIG. 22) according to the fifth embodiment.

As described above, in the fourth to sixth embodiments, the support is provided with the relatively easily-cut portions. Thus, there is an effect that cutting of the support can be easily performed. Additionally, when the support and a living body are sewn with a thread in an implantation processing, the thread is wound around the easily-cut portion, so that the thread can be prevented from moving in the axial direction. Other effects similar to those discussed above with respect to the first to third embodiments are also realized.

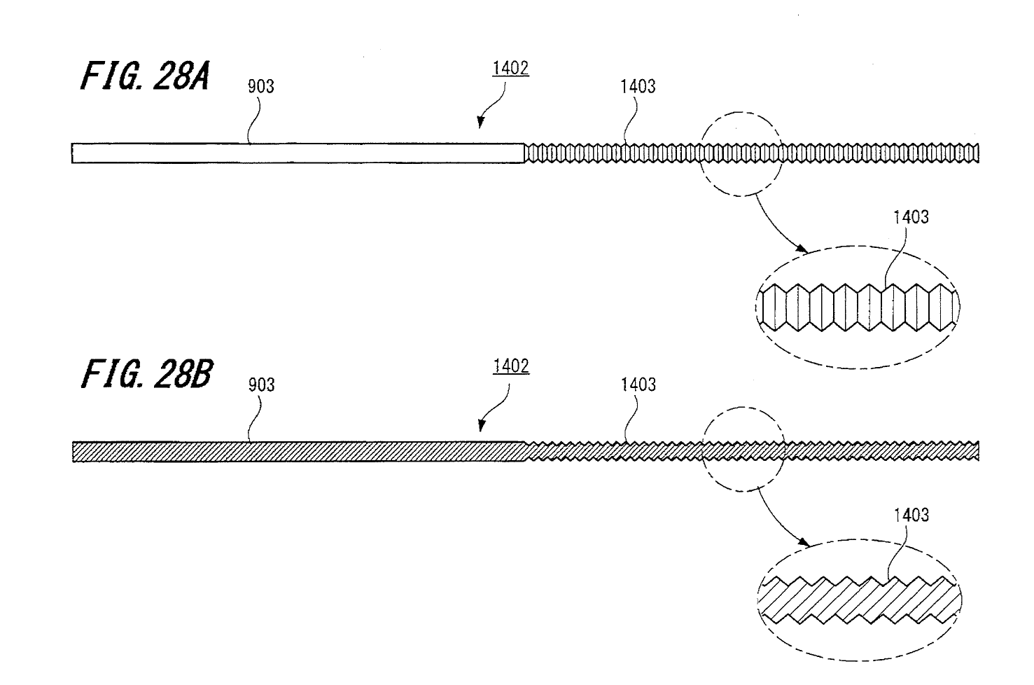

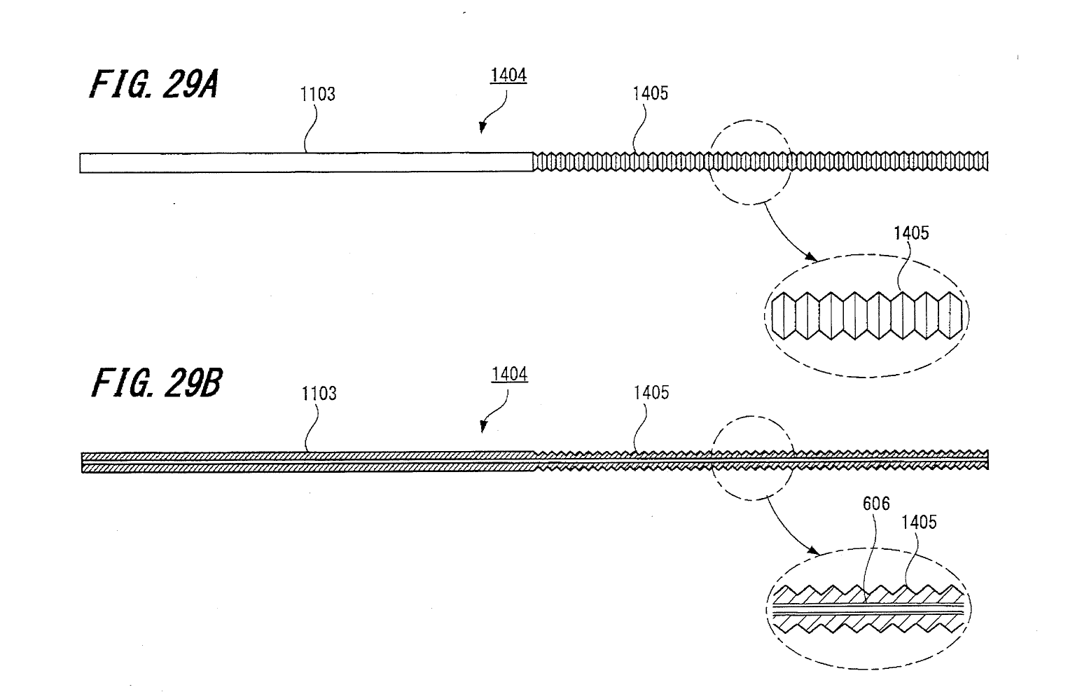

Here, a first modification of the electric stimulators according to the fourth to sixth embodiments will be described with reference to FIGS. 28A and 28B and FIGS. 29A and 29B.

The first modification provides a support 1402 instead of the support 902 of the electric stimulator 901 shown in FIG. 18. More specifically, a configuration in which a second body 1403 as shown in FIGS. 28A and 28B is provided is adopted as an alternative to the second body 904 (refer to FIG. 18) of the support.

The second body 1403 is made of the same material as the second body 904. Instead of being configured in the manner shown in FIG. 18 (a shape in which a plurality of abacus balls or relatively short cylindrical segments are arranged in the direction of a minor axis), the second body 1403 is configured as a plurality of substantial bicones arranged in the axial direction. In addition, portions of the second body 1403 with the shortest diameter have the same function as the relatively easily-cut portions 1003 (refer to FIGS. 20A and 20B) of the second body 904. That is, in order to adjust the axial length of the electric stimulator, a portion of the second body 1403 with the shortest or smallest outer diameter is cut-off, and this portion (the distal end of the remaining portion connected to the body 903) is sewn on a tissue in a living body with a thread to fix the electric stimulator.

In addition, by forming a substantially cylindrical cavity in the support 1402 in the axial direction as in a support 1404 of FIGS. 29A and 29B, it is also possible to use the support 1404 as an alternative to the support 1102 (refer to FIGS. 22 and 26) of the electric stimulators 1101 and 1301 of the fifth and sixth embodiments.

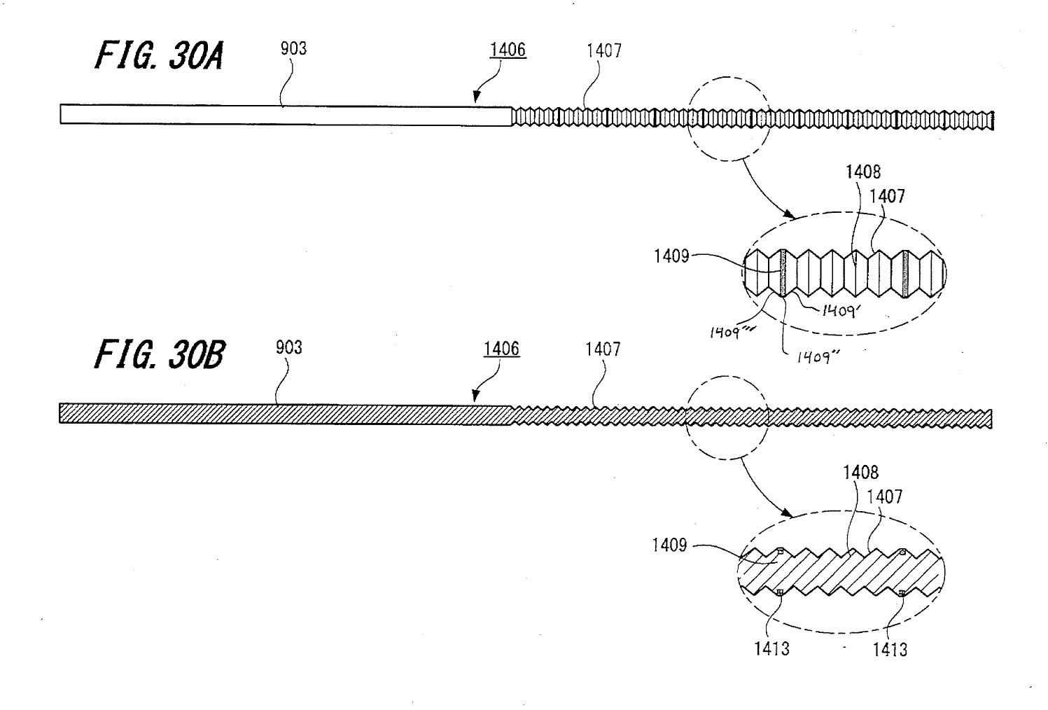

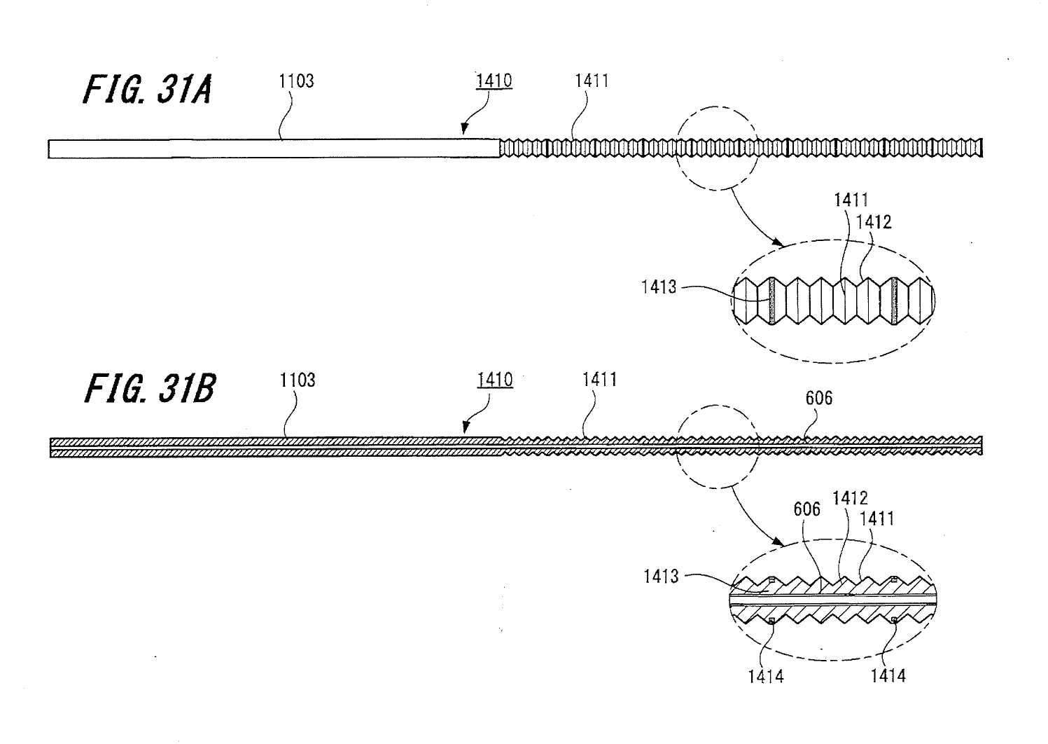

A second modification of the electric stimulators according to the fourth to sixth embodiments is illustrated in FIGS. 30A and 30B and FIGS. 31A and 31B. FIGS. 30A and 30B are explanatory views showing a support.

The second modification provides a support 1406 instead of the support 902 of the electric stimulator 901. More specifically, a configuration in which a second body 1407 shown in FIGS. 30A and 30B is provided is adopted as an alternative to the second body 904 (refer to FIG. 18) of the support 902.

The second body 1407 is also made of the same material as the second body 904, and is formed to include a plurality of biconical portions 1408 and a plurality of marking portions 1409.

The biconical portions 1408 have portions formed in the shape of a substantial bicone. Additionally, the marking portions 1409 include a first substantially conical portion 1409′, a substantially cylindrical portion 1409″ with a top face that coincides with the bottom face of the first substantially conical portion 1409′, and a second substantially conical portion 1409′″ with a bottom face that forms the undersurface of the substantially cylindrical portion 1409″. A colored ring 1413 is embedded in this substantially cylindrical portion such that the axes thereof overlap each other. In addition, as described above, the materials forming the support 1406, i.e., the materials of the marking portions 1409, are transparent materials, such as silicone or polyurethane, and so the ring 1413 can be seen therethrough.

Such marking portions 1409 are provided at predetermined axially spaced-apart intervals between the respective biconical portions 1408. A doctor can know the depth when an electric stimulator is implanted in a living body, with the position of each marking portion 1409, i.e., the see-through ring 1413 as an indication.

The portions of the second body 1407 with the smallest outer diameter, that is, connecting portions between the respective biconical portions 1408 and connecting portions between the biconical portions 1408 and the marking portions 1409 have the same function as the relatively easily-cut portions 1003 of the second body 904 (refer to FIGS. 20A and 20B). That is, in order to adjust the axial length of the electric stimulator, the portion of the second body 1407 with the shortest diameter is cut, and this portion is sewn on a tissue in a living body with a thread to fix the electric stimulator.

In addition, by forming a substantially cylindrical cavity in the support 1406 of FIGS. 30A and 30B in the axial direction as in a support 1410 of FIGS. 31A and 31B, it is also possible to use the support 1410 as an alternative to the support 1102 (refer to FIGS. 22 and 26) of the electric stimulators 1101 and 1301 of the fifth and sixth embodiments.

Additionally, performing marking on each second body of the above-described respective supports with a pigment possessing biocompatibility at every predetermined interval can be an alternative to the marking portion 1409 (refer to FIGS. 30A and 30B) or 1413 (refer to FIGS. 31A and 31B).

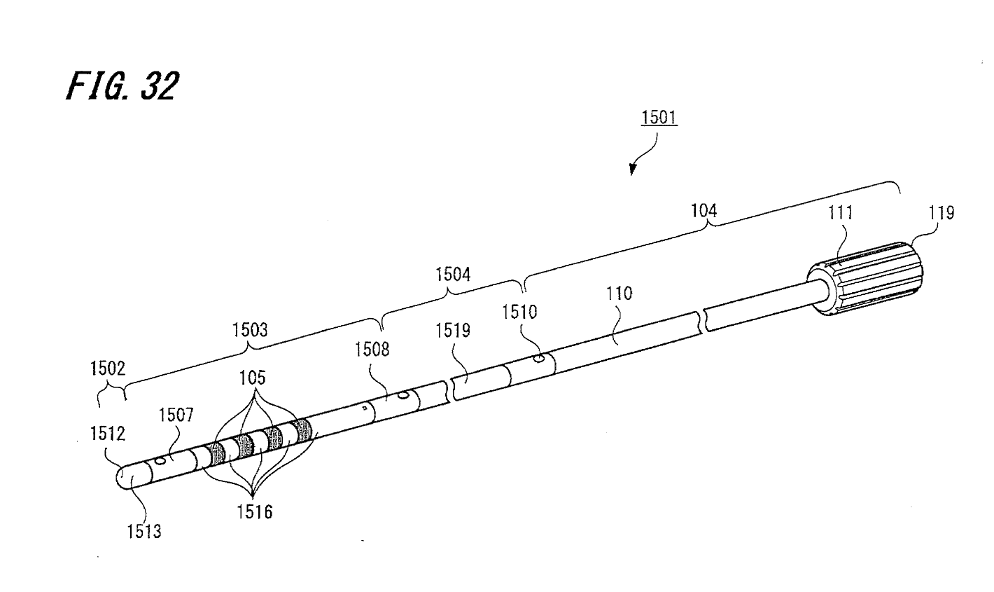

A seventh embodiment of the electric stimulator illustrated in FIGS. 32 to 35. The configuration of the electric stimulator 1501 according to the seventh embodiment shown is similar in many respects to the embodiments of the electric stimulators described above. Features and portions of this seventh embodiment of the electric stimulator that are the same as in earlier described embodiments are designated with common reference numerals, and a detailed description of such features will not be repeated.

The implantation procedure of the electric stimulator 1501 according to the seventh embodiment is the same as the implantation procedure of the electric stimulator 101 according to the first embodiment, and so a detailed description of that procedure will not be repeated here.

### Configuration of Electric Stimulator

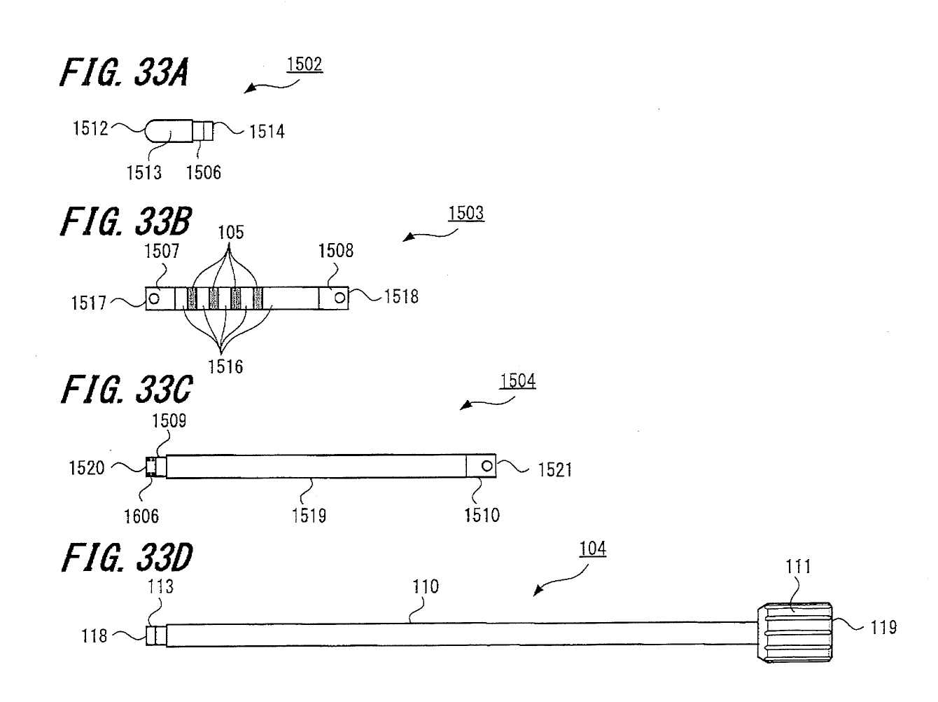

First, the general configuration of the electric stimulator according to the seventh embodiment will be described with reference to FIG. 32 and FIGS. 33A to 33D. Similar to the above-described respective electric stimulators, the electric stimulator 1501 is formed in the shape of a rod (i.e., is rod-shaped), generates an electric stimulation signal, and stimulates nerves or the like in a living body by the stimulation signal. The electric stimulator 1501, as shown in FIG. 32 and FIGS. 33A to 33D, includes an electrode block 1503 and a circuit block 1504, respectively, instead of the electrode block 102 and the circuit block 103 of the electric stimulator 101 (refer to FIG. 1) according to the first embodiment, and further includes an insertion block 1502.

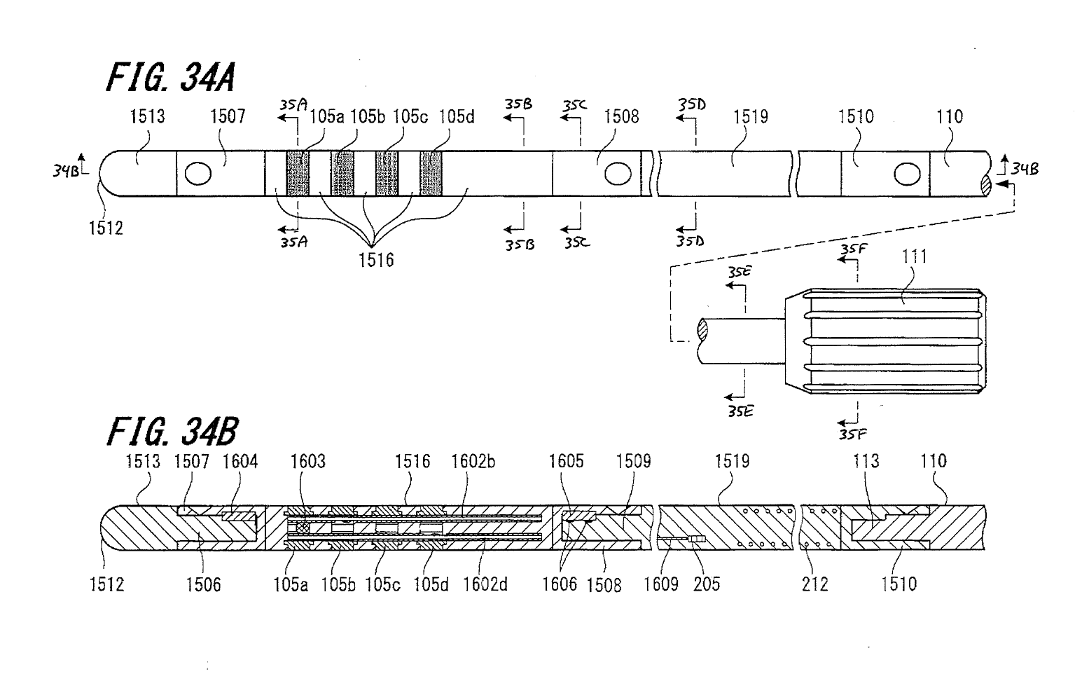

The insertion block 1502 includes a body 1513 in which a distal end 1512 is formed in a substantially hemispherical shape, and the other portions possess a substantially cylindrical shape. A connector part 1506 for connecting the insertion block 1502 to the electrode block 1503 is disposed on a proximal end 1514 of the body 1513. The internal configuration of the insertion block 1502 will be described with reference to FIGS. 34A and 34B.

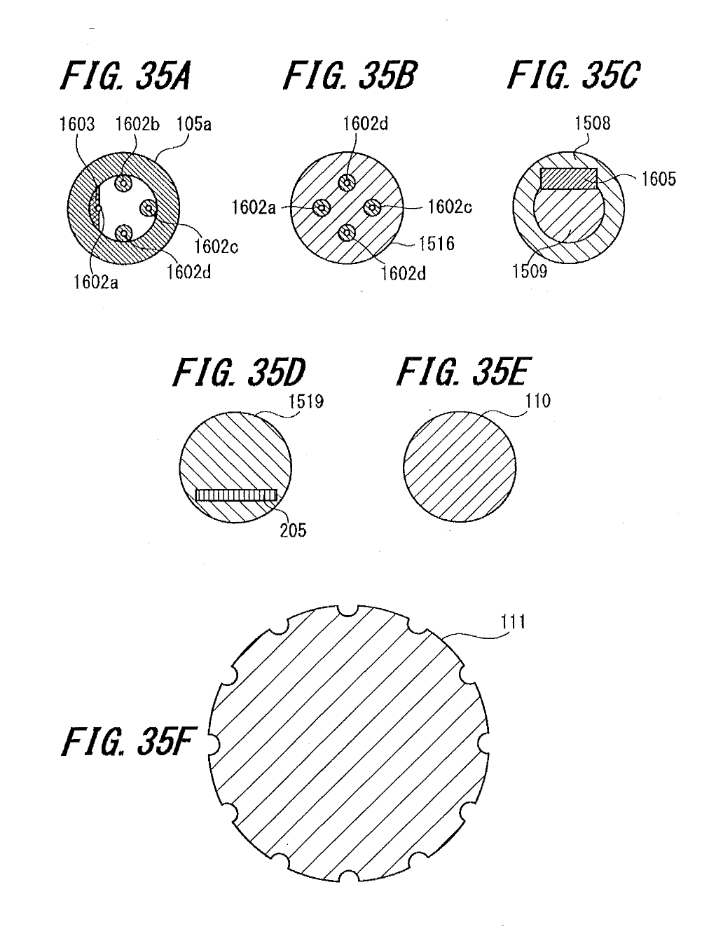

The electrode block 1503 possesses a substantially cylindrical shape with the same outer diameter as the substantially cylindrical portion of the insertion block 1502. This electrode block 1503 includes the above-described stimulation electrodes 105, and a body 1516 having portions arranged at equal axially spaced intervals such that, when the electric stimulator 1501 is implanted in a living body, the respective stimulation electrodes 105 are exposed to the living body. A connector part 1507 to be connected to the connector part 1506 of the insertion block 1502 is provided on the distal end 1517 of the body 1516 such that the distal end 1517 of the electrode block 1503 and the proximal end 1514 of the body 1513 of the insertion block 1502 are continuous with each other. Moreover, a connector part 1508 for connecting the circuit block 1504 to a proximal end 1518 of the electrode block 1503 is disposed on the proximal end 1518 side of the body 1516. The internal configuration of the electrode block 1503 will be described below with reference to FIGS. 34A and 34B and FIGS. 35A to 35F.

The circuit block 1504 possesses a substantially cylindrical shape with the same outer diameter as the outer diameter of the electrode block 1503. The circuit block 1504 includes a connector part 1509 that is connected to the connector part 1508 of the electrode block 1503 such that a distal end 1520 of a body 1519 is continuous with the proximal end 1518 of the electrode block 1503. Additionally, the circuit block 1504 is provided with the body 1519 that is continuous with the connector part 1509. Moreover, the circuit block 1504 includes a connector part 1510 that is continuous with a proximal end 1521 side of the body 1519 to connect the support 104 to the proximal end 1521 of the circuit block 1504. The internal configuration of the circuit block 1504 and the internal configuration of the electric stimulator 1501 will be described below with reference to FIGS. 34A and 34B and FIGS. 35A to 35F.

First, the internal configuration of the insertion block 1502 (refer to FIG. 33A) will be described.

The body 1513 is made of resin materials that have pliability and biocompatibility, for example, materials, such as silicone or polyurethane. The distal end 1512 of the body 1513 is substantially hemispherical as described above, and the radius thereof is preferably within a range of about 0.5 mm to 1.5 mm. Portions of the body 1513 other than the distal end 1512 possess a substantially cylindrical shape, and the diameter thereof is preferably in a range of about 1 to 3 mm.

The connector part 1506 is formed of the same material as the body 1513, and is formed as a cutout part in which a level difference is provided from the external diameter of the substantially cylindrical body 1513. That is, the outer dimension of the connector part 1506 is smaller than the remainder of the body 1513. This cutout part extends a predetermined distance in the axial direction from the proximal end 1514 (refer to FIG. 33A).

The internal configuration of the electrode block 1503 (refer to FIG. 33B) will next be described. The body 1516 is made of for example, material, such as polyurethane or silicone.

A portion of the body 1516 in the axial possesses a hollow, substantially cylindrical shape, and the external diameter of the body 1516 is equal to the diameter of the substantially cylindrical portion of the body 1513 of the insertion block 1502. The body 1516 is fixed such that the four stimulation electrodes 105a to 105d described in FIG. 3 are exposed at the outer surface of the body 1516.

Portions of conducting wires 1602a to 1602d are bonded to the stimulation electrodes 105a to 105d, respectively, with solder 1603 (refer to FIGS. 34B and 35A). One end of each of the conducting wires 1602a to 1602d is electrically connected to the connector part 1507, and the other end of each of the conducting wires 1602a to 1602d is electrically connected to the connector part 1508. In addition, locations other than the locations of the conducting wires 1602 that are bonded with the solder 1603 and the locations of the conducting wires 1602 that are electrically connected to the connector part 1507 or 1508 are insulated and coated with PTFE or ETFE and are completely embedded in the body 1516 (refer to FIG. 35B).

The connector parts 1507 and 1508 are made of the same material as the body 1516 except for electrical connecting portions 1604 and 1605 that will be described below, and possess a hollow, substantially cylindrical shape in the axial direction. The external diameter of the connector parts 1507 and 1508 is approximately equal to the external diameter of the body 1516, and the hollow portions of the connector parts 1507 and 1508 are formed in almost the same shape and size as the outer shell of the connector part 1506 of the insertion block 1502 so that the connector parts 1507 and 1508 arc respectively connectable to the connector part 1506 of the insertion block 1502 or the connector part 1509 of the circuit block 1504. Moreover, the connector parts 1507 and 1508 respectively include the electrical connecting portions 1604 and 1605 including four electrodes that are independently electrically connected to the conducting wires 1602a to 1602d, respectively.

Next, the internal configuration of the circuit block 1504 (refer to FIG. 33C) will be described.

The connector part 1509 of the circuit block 1504, similar to the connector part 1506 of the insertion block 1502, is also formed of, for example, materials, such as polyurethane or silicone. The connector part 1509, as described above, has the same shape and size as the connector part 1506 of the insertion block 1502 connectable to the connector part 1508 so as to be connectable to the connector part 1508 of the electrode block 1503. Additionally, four connector pins 1606 are exposed and arranged on a cutout part. The four connector pins 1606 of the connector part 1509 are respectively electrically connected to the four electrodes of the electrical connecting portion 1605 of the connector part 1508, in a state of the connector part 1509 being connected to the connector part 1508 of the electrode block 1503.

The body 1519 is made of the same material as the connector part 1509. The body 1519 possesses a substantially cylindrical shape, and is sized and configured such that the outer diameter thereof is approximately equal to the external diameter of the body 1516 of the electrode block 1503.

Additionally, the stimulation circuit 205 described in FIGS. 3B and 5 and the coil portion 212 electrically connected to the stimulation circuit 205 are embedded in the body 1519. In addition, the coil portion 212 is wound in the axial direction.

The stimulation circuit 205 is respectively connected to the four connector pins 1606 of the connector part 1509 via conducting wires 1609 embedded in the body 1519 in order to supply a generated electric stimulation signal to each stimulation electrode 105, independently.

The connector part 1510 has the same shape and size as the connector parts 1507 and 1508 of the electrode block 1503, and all of these are made of the same material as the body 1519.

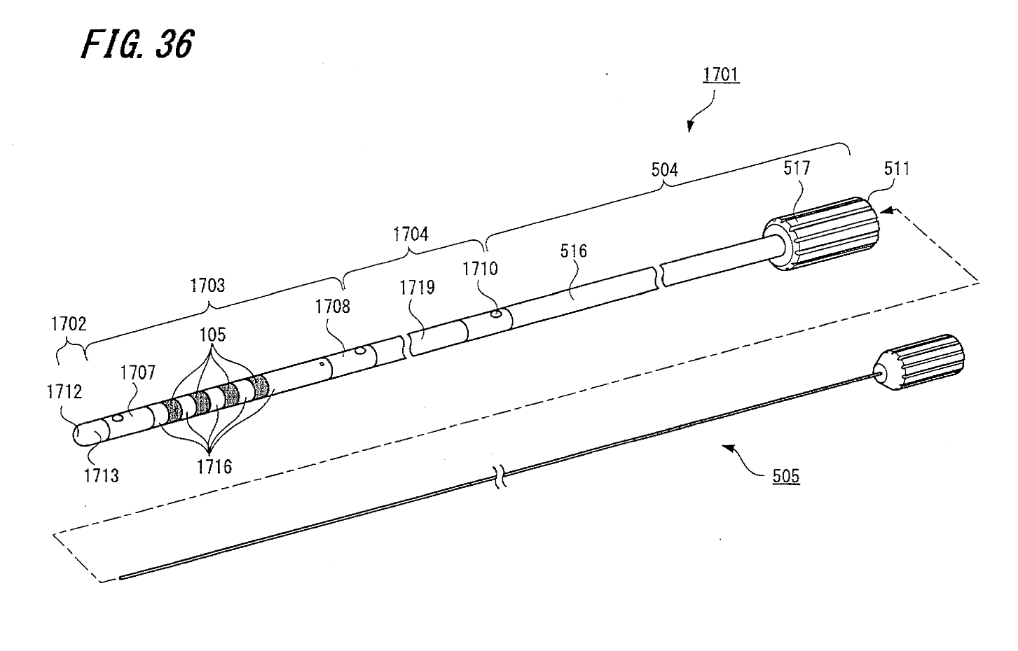

An eighth embodiment of the stimulation electrode is illustrated in FIGS. 36 to 39. The electric stimulator 1701 according to the eighth embodiment shown in FIGS. 36 to 39 is similar to the seventh embodiment of the stimulation electrode, but additionally includes a lumen for a stylet. Because the configuration of the electric stimulator according to this eighth embodiment is similar in many respects to embodiments of the electric stimulators described above, a detailed description of the entire stimulation electrode is not repeated. Features and portions of this eighth embodiment of the electric stimulator that are the same as in earlier described embodiments are designated with common reference numerals. The implantation procedure of the electric stimulator according to this eighth embodiment is generally the same as the implantation procedure of the electric stimulator 501 according to the second embodiment, and so a detailed description of the entire implantation procedure is also not repeated.

### Configuration of Electric Stimulator

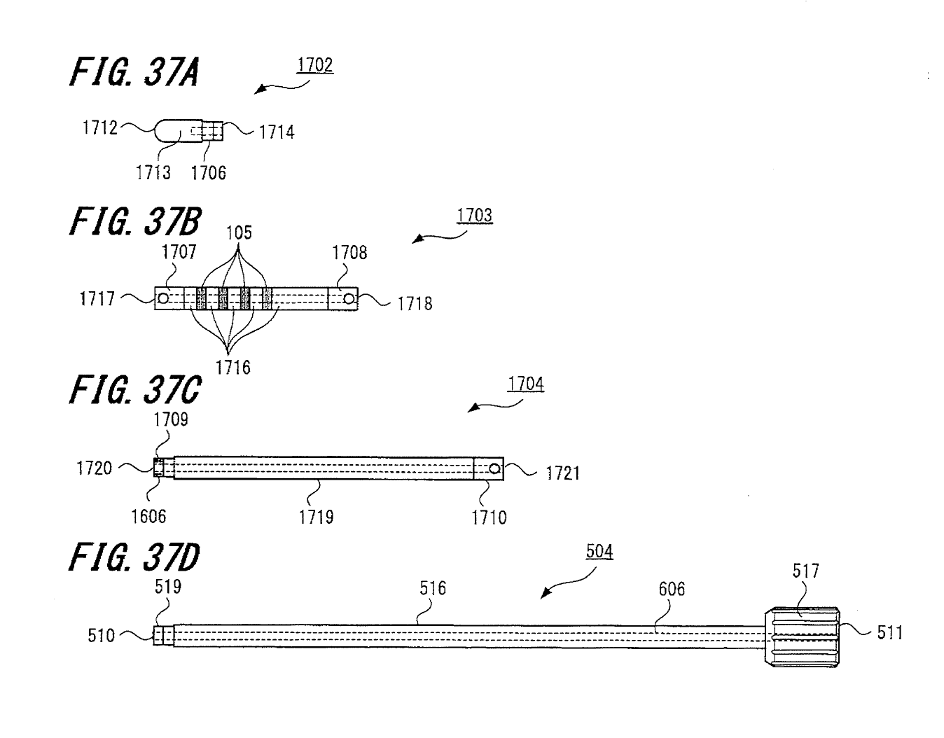

The general configuration of the electric stimulator according to the eighth embodiment will be described with reference to FIG. 36 and FIGS. 37A to 37D.

Like the above-described electric stimulators, the electric stimulator 1701 is formed in the shape of a rod (i.e., is rod-shaped), generates an electric stimulation signal, and stimulates nerves or the like in a living body by the stimulation signal. As described above, this embodiment is similar to the seventh embodiment, except an axially extending lumen for a stylet is provided in the electric stimulator 1701. Therefore, the electric stimulator 1701 is constituted by an insertion block 1702, an electrode block 1703, a circuit block 1704, and the support 504 that form a hollow portion in the axial direction, instead of the insertion block 1502, the electrode block 1503, the circuit block 1504, and the support 104 shown in FIG. 32. Since the support 504 has been described in the second embodiment, the description of the support 504 is not repeated.

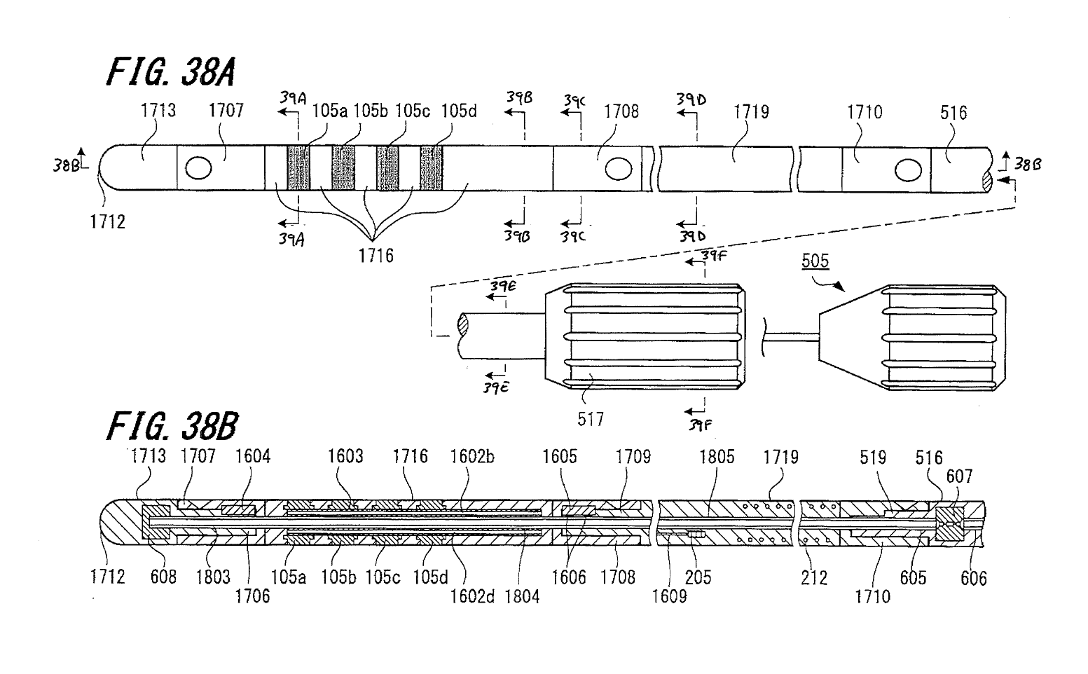

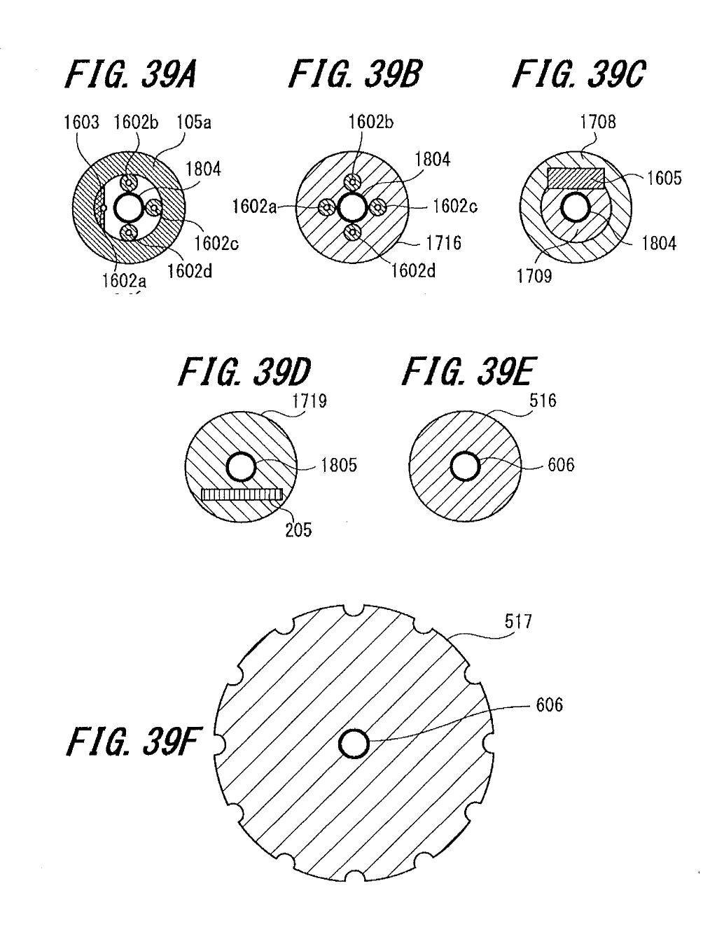

In the electric stimulator 1701, as shown in FIGS. 37A to 37D, when the respective blocks are connected together in order of the insertion block 1702, the electrode block 1703, the circuit block 1704, and the support 504, these blocks together form or include a portion of the lumen for a stylet that communicates in the axial direction of the blocks. The stylet lumen opens to the rear of the stimulation electrode by virtue of an opening provided in the proximal end 511, and this opening is connected or continuous up to the vicinity of a distal end 1712. In addition, the inner diameter of the lumen for the stylet is preferably approximately equal to or slightly greater than the outer diameter of the stylet 505. The internal configuration of the insertion block 1702, the electrode block 1703, and the circuit block 1704 will be described below in FIGS. 38A and 38B and FIGS. 39A to 39F.

Next, the internal configuration of the electric stimulator related to the eighth embodiment will be described with reference to FIGS. 38A and 38B and FIGS. 39A to 39F. First, the internal configuration of the insertion block 1702 will be described.

A pipe 1803 is made of materials that have biocompatibility, insulation, and pliability, for example, PTFE or ETFE, and is formed in a hollow, substantially cylindrical shape in the axial direction. It is desirable that the external diameter of the pipe 1803 be about 0.1 to 1 mm, and the internal diameter of the pipe 1803 be approximately equal to or slightly longer than the diameter of the stylet 505 so that the stylet 505 can pass through the inside of the pipe 1803. One end (end at the distal end 1712) of such a pipe 1803 is connected to the receiving portion 608 described in FIG. 13B. The pipe 1803 and the receiving portion 608 are housed in and fixed to an outer layer part made up of a body 1713, and a connector part 1706 (refer to FIG. 38B).

The body 1713 is made of resin materials that have pliability and biocompatibility, for example, materials, such as silicone or polyurethane. The distal end 1712 of the body 1713 is substantially hemispherical, and the radius thereof is preferably within a range of about 0.5 mm to 1.5 mm. Portions of the body 1713 other than the distal end 1712 are formed in a substantially cylindrical shape that is hollow in the axial direction. The external diameter of the hollow, substantially cylindrical portion is desirably in a range of about 1 to 3 mm, and the internal diameter is approximately equal to the external diameter of the receiving portion 608 or the pipe 1803, in order to house and fix the receiving portion 608 and the pipe 1803.

The connector part 1706 is formed of the same material as the body 1713, and is formed as a cutout part in which a level difference is provided from the external diameter of the hollow, substantially cylindrical body 1713. This cutout part is formed by a predetermined distance in the axial direction from a proximal end 1714 (refer to FIG. 37A).

Next, the internal configuration of the electrode block 1703 (refer to FIG. 37B) will be described.

A pipe 1804 that constitutes the electrode block 1703 is also the same as the pipe 1803, except for the axial length of the pipe 1804. The pipe 1804 is housed in and fixed to an outer layer part including a connector part 1707, a body 1716, the stimulation electrodes 105, and a connector part 1708 (refer to FIG. 38B).

The body 1716 is made of the same material as the body 1713, and is formed in a hollow, substantially cylindrical shape in the axial direction. The external diameter of the body 1716 is equal to the external diameter of the hollow, substantially cylindrical portion of the body 1713 of the insertion block 1702, and the internal diameter of the body 1716 is approximately equal to the external diameter of the pipe 1804 in order to house and fix the pipe 1804. The above-described four stimulation electrodes 105a to 105d are exposed at the surface of the body 1716.

The connector parts 1707 and 1708 are made of the same material as the body 1716, except for the above-described electrical connecting portions 1604 and 1605. The external diameter of the connector parts 1707 and 1708 is approximately equal to the external diameter of the body 1716, and the hollow portions formed in the connector parts 1707 and 1708 possess almost the same shape and size as the outer shell of the connector part 1706 of the insertion block 1702 so that the connector parts 1707 and 1708 are respectively connectable to the connector part 1706 of the insertion block 1702 or a connector part 1709 of the circuit block 1704. Moreover, the connector parts 1707 and 1708 respectively include the electrical connecting portions 1604 and 1605 including four electrodes as described above.

Next, the internal configuration of the circuit block 1704 (refer to FIG. 37C) will be described.

A pipe 1805 is the same as the pipe 1803, except for the axial length of the pipe 1805. The pipe 1805 is housed in and fixed to an outer layer part including the connector part 1709, a body 1719, and a connector part 1710 (refer to FIG. 38B).

The connector part 1709 is made of the same material as the connector parts 1706 to 1708. The connector part 1709 has the same shape and size as the connector part 1706 of the insertion block 1702 connectable to the connector part 1708 so as to be connectable to the connector part 1708 of the electrode block 1703, and the four connector pins 1606 are exposed and arranged on a cutout part or reduced outer diameter part (refer to FIG. 37C). The four connector pins 1606 of the connector part 1709 are respectively electrically connected to the four electrodes of the electrical connecting portion 1605 of the connector part 1708, in a state of the connector part 1709 being connected to the connector part 1708 of the electrode block 1703.

The body 1719 of the circuit block 1704 is made of the same material as the connector part 1709. The body 1719 possesses a hollow, substantially cylindrical shape in the axial direction. The external diameter of the body 1719 is approximately equal to the external diameter of the body 1716 of the electrode block 1703, and the internal diameter of the body 1719 is approximately equal to the external diameter of the pipe 1805 in order to house and fix the pipe 1805.

The stimulation circuit 205 described in FIG. 3B and the coil portion 212 electrically connected to the stimulation circuit 205 are embedded in the body 1719. In addition, the coil portion 212 is wound in the axial direction. The stimulation circuit 205 is respectively connected to the four connector pins 1606 of the connector part 1709 via the conducting wires 1609 embedded in the body 1719 in order to be able to supply a generated electric stimulation signal to each stimulation electrode 105, independently. In addition, the connector part 1710 has the same shape and size as the connector parts 1707 and 1708 of the electrode block 1703, and all of these are made of the same material as the body 1719.

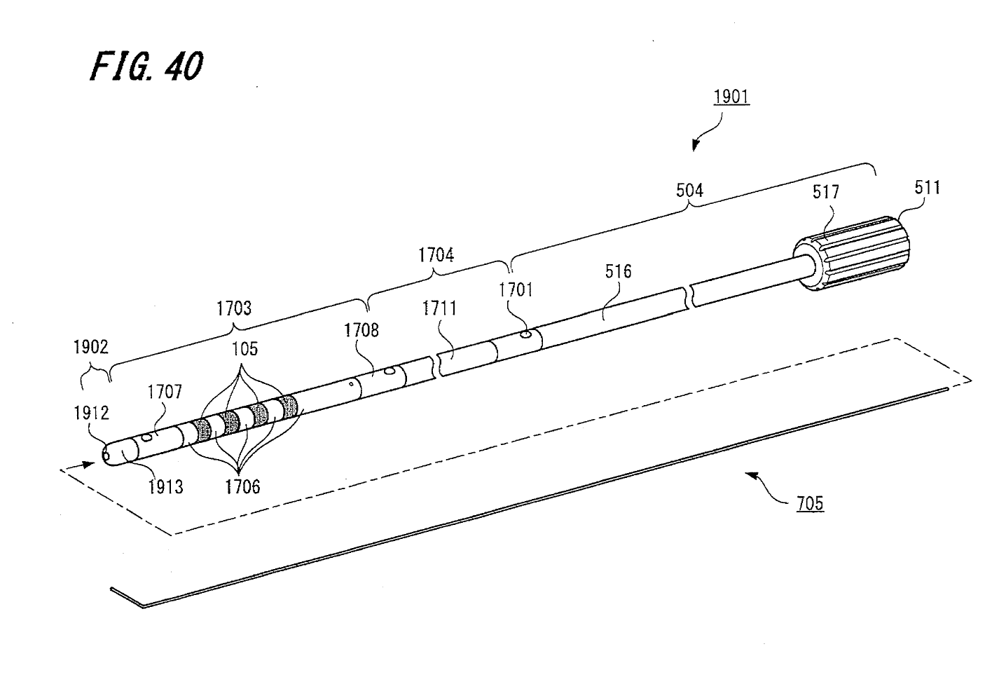

A ninth embodiment of the stimulation electrode is illustrated in FIGS. 40 to 42. An electric stimulator 1901 according to the ninth embodiment shown in FIGS. 40 to 42 is similar to the seventh embodiment of the stimulation electrode, except this ninth embodiment includes a lumen for a guide wire in the electric stimulator 1501. Features and portions of this embodiment of the electric stimulator that are the same as in earlier described embodiments are designated with common reference numerals, and a detailed description of such portions and features is not repeated. The implantation procedure of the electric stimulator 1901 according to this ninth embodiment is the same as the implantation procedure of the electric stimulator 701 described above, and so a detailed description of the implantation procedure is also not repeated.

### Configuration of Electric Stimulator

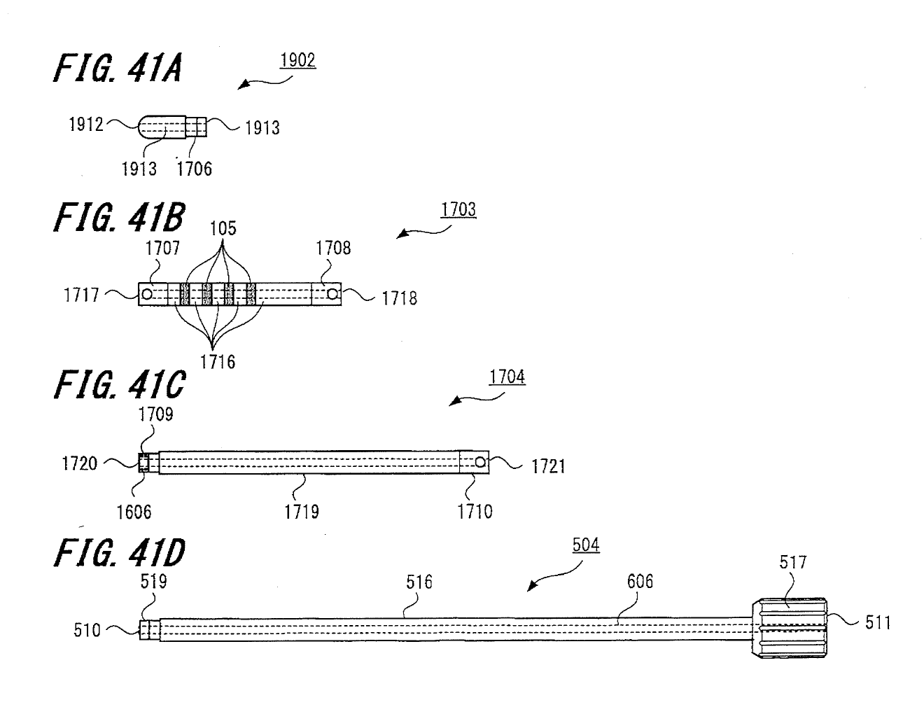

First, the general configuration of the electric stimulator according to the ninth embodiment will be described with reference to FIG. 40 and FIGS. 41A to 41D.

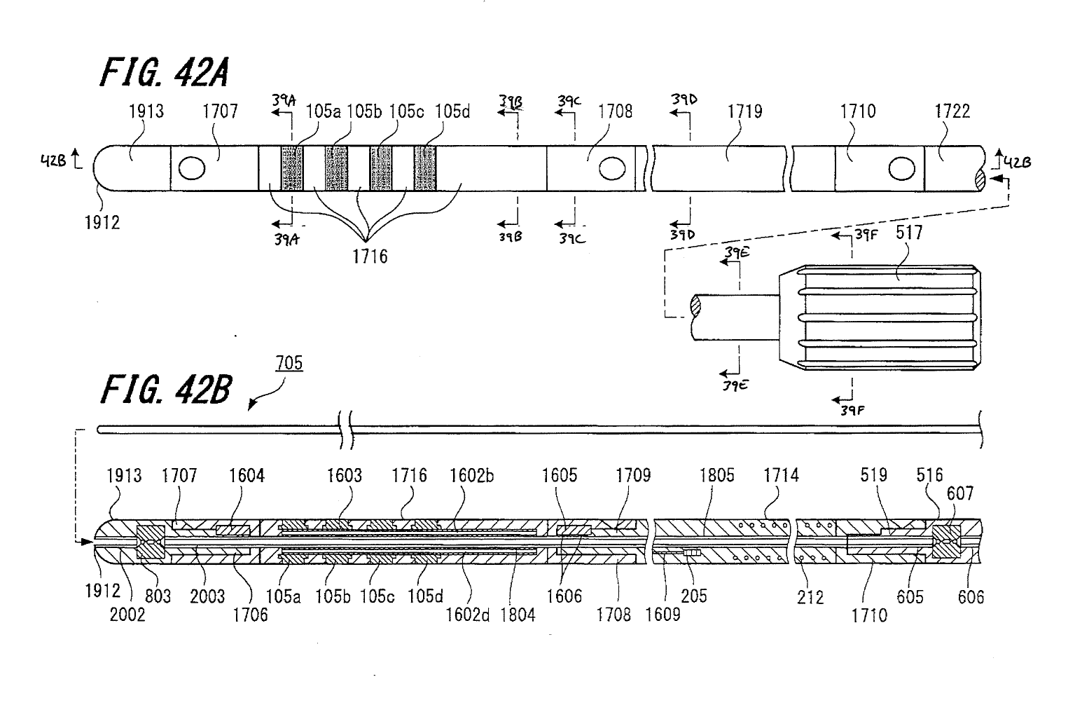

Similar to the above-described respective electric stimulators, the electric stimulator 1901 is formed in the shape of a rod (i.e., is rod-shaped), generates an electric stimulation signal, and stimulates nerves or the like in a living body by the stimulation signal. Since the electric stimulator 1901 has the axially extending lumen for a guide wire as described above, the electric stimulator 1901 possesses a hollow, substantially cylindrical shape. The lumen for a guide wire, as shown in FIGS. 41A to 41D, is provided as a through hole that opens to the proximal end 511 and also opens to the distal end 1912. Therefore, the electric stimulator 1901 has a configuration in which the insertion block 1702 of the electric stimulator 1701 of the eighth embodiment is replaced with a substantially cylindrical insertion block 1902 possessing a hollow portion (refer to FIG. 41A) that communicates in the axial direction and extends throughout the entirety of the insertion block 1902. The internal configuration of the insertion block 1902 will be described with reference to FIGS. 42A and 42B.

Next, the internal configuration of the electric stimulator according to the ninth embodiment will be described with reference to FIGS. 42A and 42B and FIGS. 39A to 39F. Although FIGS. 39A to 39F, as described above, are cross-sectional views showing the internal structure at predetermined locations in a vertical direction (perpendicular direction) with respect to the axis of the electric stimulator 1701 according to the eighth embodiment of the stimulation electrode, FIGS. 39A to 39F are also cross-sectional views showing the internal structure in a vertical direction with respect to the electric stimulator 1901 according to the ninth embodiment.

Here, only the internal configuration of the insertion block 1902 will be described.

Pipes 2002 and 2003 are the same as the pipes 1804 and 1805, except for the axial length of the pipes. The valve 803 described in FIG. 17B is provided between the pipe 2002 and the pipe 2003 in the axial direction. When the guide wire 705 is inserted and removed via the valve 803, liquids, such as body fluids, can be prevented from entering the insides of the electrode block 1703 and the circuit block 1704 from the hole provided in the distal end 1912 of a body 1913.

The pipes 2002 and 2003 and the valve 803 are housed and fixed in an outer layer part made up of the body 1913, and the connector part 1706.

Although the body 1913 is made of, for example, materials, such as silicone or polyurethane, the body 1913 has a substantially cylindrical hole in the distal end 1912 thereof. The internal diameter of this hole is approximately equal to the external diameter of the pipe 2002. In addition, the external diameter of the body 1913 is the same as that of the body 1713 (refer to FIGS. 38A and 38B) of the eighth embodiment. The connector part 1706 is the same as the connector part described in the eighth embodiment, and so a detailed description of the connector part 1706 is not repeated.

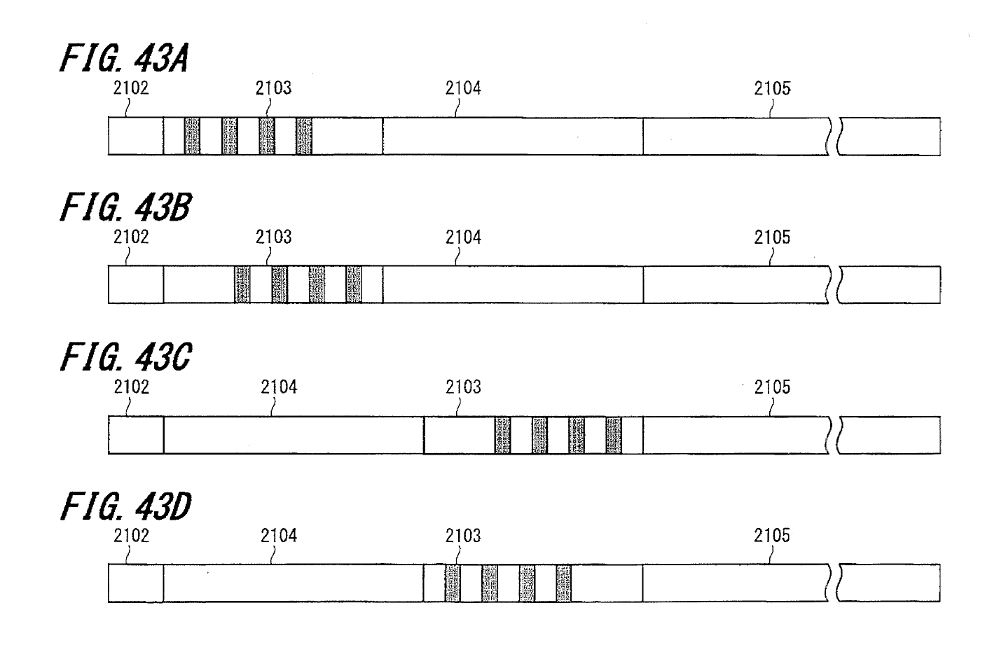

In the above-described seventh to ninth embodiments, the electric stimulators 1501, 1701, and 1901, as shown in FIG. 43A, are connected in order of a insertion block 2102, a electrode block 2103, a circuit block 2104, and a support 2105 from a distal end side. However, respective connector parts provided at both ends of the electrode block 2103 of the respective embodiments have the same shape and size (refer to FIGS. 34B, 38B, and 42B). Therefore, the electrode block 2103 illustrated in FIG. 43A can be reversed in the horizontal direction, so that the insertion block 2102, the electrode block 2103, the circuit block 2104, and the support 2105 are mechanically connected in order from the distal end as depicted in FIG. 43B. Since the connector parts at both ends of the electrode block 2103 respectively have the electrical connecting portions electrically connected to the stimulation electrodes (refer to FIGS. 34B, 38B, and 42B), even if the respective blocks are connected as shown in FIG. 43B, an electric stimulation signal generated in the circuit block 2104 (more specifically, the stimulation circuit) is applied to the stimulation electrodes of the electrode block 2103. That is, if the electrode block 2103 and the circuit block 2104 are mechanically connected in the manner shown in FIG. 43B, the electrode block 2103 and the circuit block 2104 can be electrically connected.

Additionally, the shape of one connector part (the connector part that is not connected to the electrode block 2103) of the circuit block 2104 is the same as that of the connector parts at both ends of the electrode block 2103. Accordingly, as shown in FIG. 43C or FIG. 43D, the respective blocks are mechanically connected in order of the insertion block 2102, the circuit block 2104, the electrode block 2103, and the support 2105 from the distal end side. The circuit block 2104 and the electrode block 2103 are electrically connected as described, even if the electric stimulator is in the state of FIG. 43C or 43D.

As described above, since the electric stimulators of the above-described seventh to ninth embodiments can change the connection between the respective blocks as in FIGS. 43A to 43D, the position of the stimulation electrodes can be changed. Therefore, the position of the stimulation electrodes can be still more freely determined than when adjusting the length of only the support to determine the position the stimulation electrodes. That is, the electric stimulator can be completely implanted in a living body, holding the position of the stimulation electrodes without any further restrictions on patient's bodily features. Thereby, suitable nerves can be stimulated more reliably. In addition, effects similar to those described above with respect to the first to third embodiments are also realized with this embodiment.

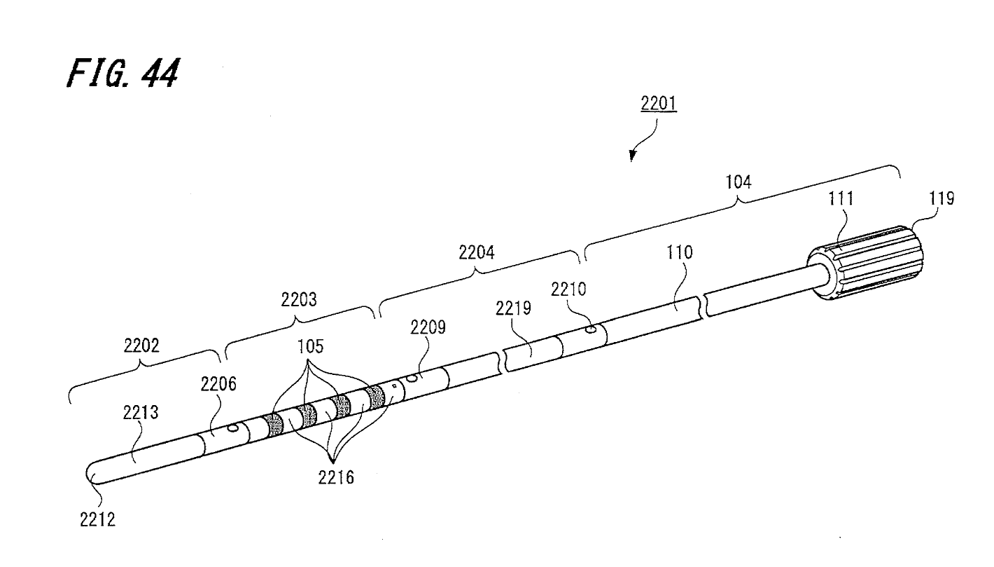

FIGS. 44 to 47 illustrate a tenth embodiment of the stimulation electrode. The configuration of this embodiment of the electric stimulator 2201 is similar to earlier described embodiments and so common features are designated by common reference numerals, and a detailed description of all of such features will not be repeated. The implantation procedure of the electric stimulator 2201 according to this embodiment is the same as the implantation procedure of the electric stimulator 101 according to the first embodiment described above, and so a detailed description of the implantation procedure is also not repeated.

### Configuration of Electric Stimulator

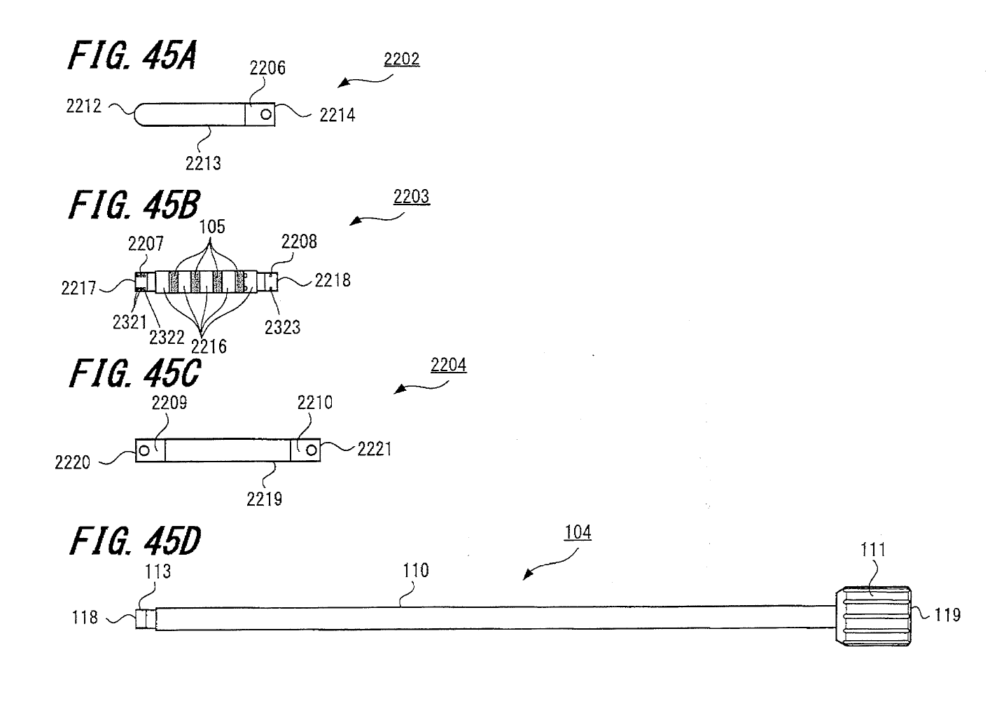

First, the general configuration of the electric stimulator according to the tenth embodiment will be described with reference to FIG. 44 and FIGS. 45A to 45D.

Like the above-described respective electric stimulators, the electric stimulator 2201 is formed in the shape of a rod (i.e., is rod-shaped), generates an electric stimulation signal, and stimulates nerves or the like in a living body by the stimulation signal. The electric stimulator 2201, as shown in FIG. 44, includes a circuit block 2202, an electrode block 2203, and a coil block 2204, instead of the electrode block 102 and the circuit block 103 of the electric stimulator 101 (refer to FIG. 1) related to the first embodiment.

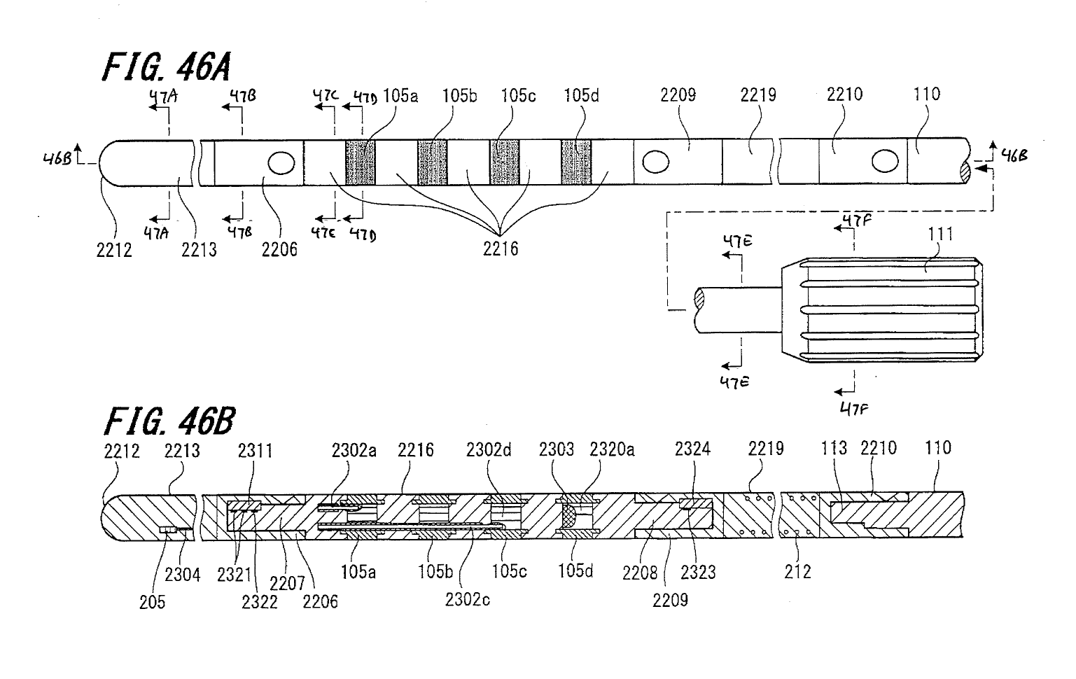

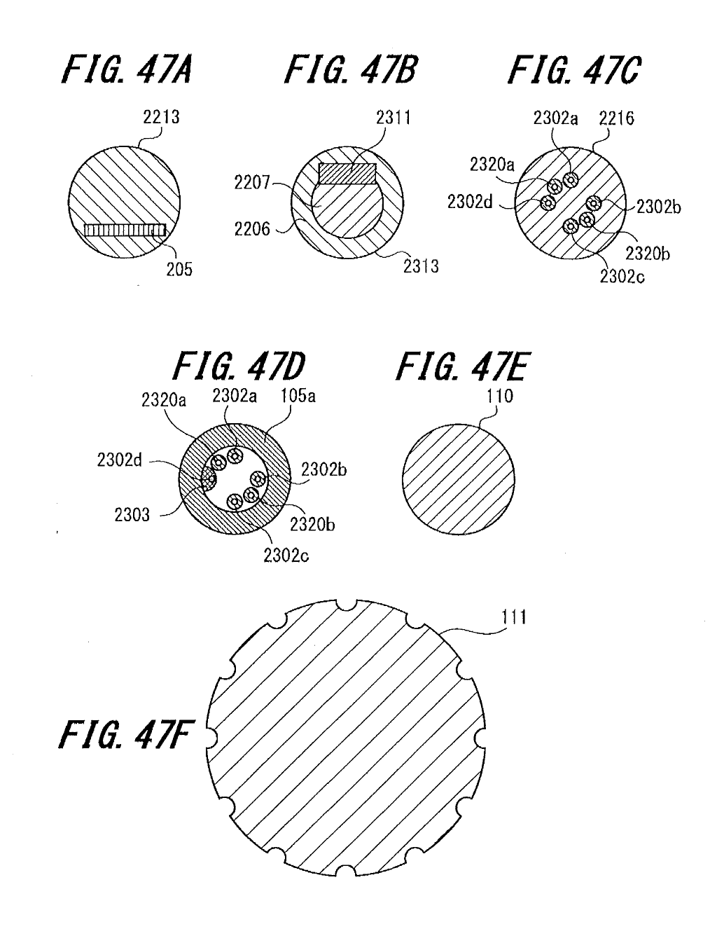

The circuit block 2202 includes a body 2213 in which a distal end 2212 possesses a substantially hemispherical shape, and the other portions possess a substantially cylindrical shape. A connector part 2206 for connecting the circuit block 2202 to the electrode block 2203 is disposed on a proximal end 2214 of the body 2213. The internal configuration of the circuit block 2202 will be described below with reference to FIGS. 46A and 46B and FIGS. 47A to 47F.

The electrode block 2203 possesses a substantially cylindrical shape with the same outer diameter as the outer diameter of the substantially cylindrical portion of the circuit block 2202. This electrode block 2203 includes the above-described stimulation electrodes 105, and a body 2216 configured so that portions of the body 2216 are exposed at equal intervals such that, and so that when the electric stimulator 2201 is implanted in a living body, the respective stimulation electrodes 105 are exposed to the living body. A connector part 2207 to be connected to the connector part 2206 of the circuit block 2202 is provided on a distal end 2217 of the body 2216 such that the distal end 2217 of the body 2216 and the connector part 2206 of the circuit block 2202 are continuous with each other. Moreover, a connector part 2208 for connecting the coil block 2204 to a proximal end 2218 of the electrode block 2203 is disposed on the proximal end 2218 side of the body 2216. The internal configuration of the electrode block 2203 will be described below with reference to FIGS. 46A and 46B and FIGS. 47A to 47F.

The coil block 2204 possesses a substantially cylindrical shape with the same outer diameter as the outer diameter of the electrode block 2203. The coil block 2204 includes a connector part 2209 that is connected to the connector part 2208 of the electrode block 2203 such that a distal end 2220 is continuous with the proximal end 2218 of the body 2216 of the electrode block 2203. Additionally, the coil block 2204 is provided with a body 2219 that is continuous with the connector part 2209. Moreover, the coil block 2204 includes a connector part 2210 that is continuous with a proximal end 2221 side of the body 2219 to connect the support 104 to the proximal end 2221 of the coil block 2204. The internal configuration of the electric stimulator according to the tenth embodiment, including the coil block 2204, will be described below with reference to FIGS. 46A and 46B and FIGS. 47A to 47F. First, the internal configuration of the circuit block 2202 will be described.

The body 2213 is made of resin materials that have pliability and biocompatibility, for example, materials, such as silicone or polyurethane. The distal end 2212 of the body 2213 is substantially hemispherical as described above, and the radius thereof is preferably within a range of about 0.5 mm to 1.5 mm. Portions of the body 2213 other than the distal end 2212 possess a substantially cylindrical shape, and the diameter thereof is preferably in a range of about 1 to 3 mm.

The stimulation circuit 205 described in FIG. 3B, and conducting wires 2304 that electrically connect the stimulation circuit 205 and the connector part 2206 are embedded in the body 2213, (refer to FIGS. 46B and 47A). In addition, the conducting wires 2304 include conducting wires for electric supply for obtaining electric power for generating an electric stimulation signal from the coil block 2204, and conducting wires for a stimulation signal for applying the generated electric stimulation signal independently to the respective stimulation electrodes 105.

The connector part 2206 is made of the same material as the body 2213 except for an electrical connecting portion 2311 that will be described below, and is formed in a hollow, substantially cylindrical shape in the axial direction. The external diameter of the connector part 2206 is approximately equal to the external diameter of the body 2213, and the hollow portion of the connector part 2206 is formed in almost the same shape and size as the outer shell of the connector part 2207 so that the connector part 2206 is connectable to the connector part 2207 of the electrode block 2203. Moreover, the connector part 2206 includes the electrical connecting portion 2311 electrically connected to the conducting wires 2304, i.e., the conducting wires for electric supply, and the conducting wires for a stimulation signal. The shape of the outer shell of the connector part 2207 will be described below along with the internal configuration of the electrode block 2203.

The body 2216 is made of the same material as the body 2213 of the circuit block 2202. A portion of the body 2216 in the axial direction possesses a hollow, substantially cylindrical shape, and the external diameter of the body 2216 is equal to the external diameter of the substantially cylindrical portion of the body 2213 of the circuit block 2202. The four stimulation electrodes 105 are fixed so as to be exposed at the surface of the body 2216.

One end of each of the conducting wires 2302a to 2302d is bonded to a respective one of the stimulation electrodes 105a to 105d with solder 2303 (refer to FIGS. 46B and 47D). The other end of each of the conducting wires 2302a to 2302d is electrically connected to the connector part 2207. In addition, locations other than the locations of the conducting wires 2302 that are bonded with the solder 2303 and the locations of the conducting wires 2302 that are electrically connected to the connector part 2207 are insulated and coated with PTFE or ETFE and are completely embedded in the body 2216 (refer to FIG. 47C).

The connector part 2207 is formed of the same material as the body 2216, and is formed as a cutout part (reduced outer diameter part) in which a level difference exists between the external diameter of the substantially cylindrical body 2216 (refer to FIG. 47B). This cutout part extends a predetermined distance in the axial direction from the distal end 2217 (refer to FIG. 45B). Additionally, six connector pins are exposed and arranged on the cutout part (refer to FIGS. 45B and 46B). Four of the connector pins are connector pins 2321 for a stimulation electrode electrically connected independently to the stimulation electrodes 105a to 105d via the conducting wires 2302a to 2302d, respectively, and two connector pins are connector pins 2322 for electric supply electrically connected to the connector part 2208 via conducting wires 2320a and 2320b. When the connector part 2207 is connected to the connector part 2206 of the circuit block 2202, the connector pins 2321 for a stimulation electrode are electrically connected to the conducting wires for a stimulation signal of the conducting wires 2304 via the electrical connecting portion 2311, and similarly the connector pins 2322 for electric supply are connected to the conducting wires for electric supply of the conducting wires 2304 via the electrical connecting portion 2311.

On the other hand, the connector part 2208 is made of the same material as the connector part 2207, and is formed in the same shape and size. The connector part 2208 is arranged such that two connector pins 2323 are exposed onto a cutout part or reduced outer diameter part, differently from the connector part 2207 (refer to FIGS. 45B and 46B). The two connector pins 2323 are electrically connected to the connector pins 2322 for electric supply of the connector part 2207 via the conducting wires 2320, respectively. That is, when the connector part 2207 is connected to the connector part 2206 of the circuit block 2202, the two connector pins 2323 are connected to the stimulation circuit 205 via the electrical connecting portion 2311 and the conducting wires for electric supply of the conducting wires 2304.

The internal configuration of the coil block 2204 (refer to FIG. 45C) will now be described. The body 2219 is made of the same material as the body 2216 of the electrode block 2203.

The body 2219 possesses a substantially cylindrical shape, and the outer diameter thereof is approximately equal to the external diameter of the body 2216 of the electrode block 2203. The coil portion 212 is wound and extends in the axial direction, and is embedded in the body 2219. In addition, the coil portion 212 is electrically connected to the connector part 2209 on the distal end 2220 of the body 2219.

The connector part 2209 is made of the same material as the body 2219 except for an electrical connecting portion 2324 that will be described below, and possesses a hollow, substantially cylindrical shape in the axial direction. The external diameter of the connector part 2209 is approximately equal to the external diameter of the body 2219, and the hollow portion of the connector part 2209 possesses almost the same shape and size as the outer shell of the connector part 2208 so that the connector part 2209 is connectable to the connector part 2208 of the electrode block 2203.

The connector part 2209 further includes the electrical connecting portion 2324 independently electrically connected to one end and the other end of the coil portion 212, respectively. When the connector part 2209 and the connector part 2208 of the electrode block 2203 are connected together and the connector part 2206 of the circuit block 2202 and the connector part 2207 of the electrode block 2203 are connected together, the coil portion 212 is electrically connected to the stimulation circuit 205 of the circuit block 2202. In addition, the connector part 2210 is the same shape and size as the connector part 2209, and both are made of the same material as the body 2219.

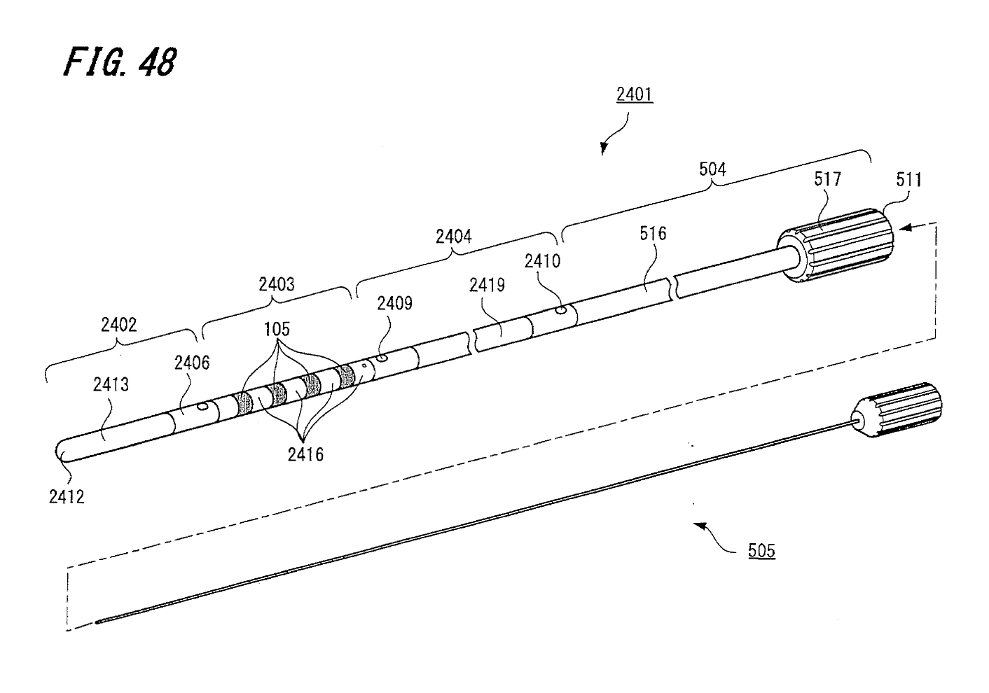

An eleventh embodiment of the invention will be described with reference to FIGS. 48 to 51. This embodiment of the electric stimulator 2401 shown in FIGS. 48 to 51 is similar to the tenth embodiment of electric stimulator 2201, but also includes a lumen for a stylet. Features of this embodiment that are the same as in earlier described embodiments are designated by common reference numerals, and a detailed description of such features is not repeated. The implantation procedure of the electric stimulator 2401 according to this eleventh embodiment is the same as the implantation procedure of the electric stimulator 501 according to the second embodiment described above, and so a detailed description of the implantation procedure is also not repeated.

### Configuration of Electric Stimulator

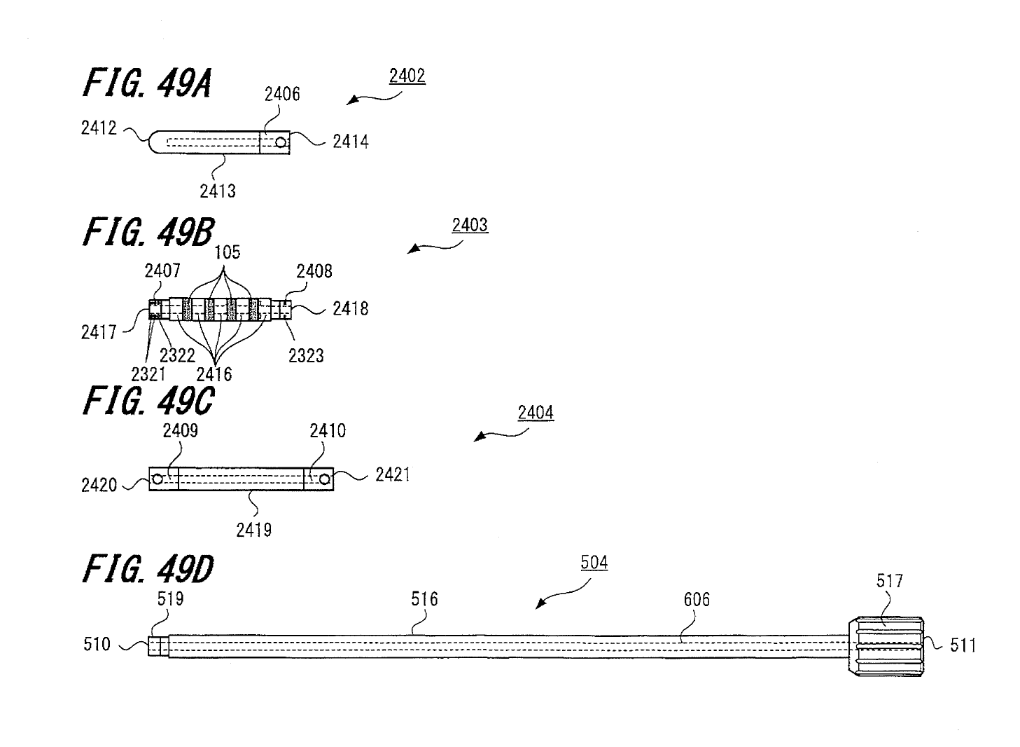

First, the general configuration of the electric stimulator according to the eleventh embodiment is described below with reference to FIG. 48 and FIGS. 49A to 49D.

The electric stimulator 2401 is configured like the electric stimulator 2201 of the tenth embodiment, but also includes the lumen for a stylet extending in the axial direction. The electric stimulator 2401 is thus constituted by a circuit block 2402, an electrode block 2403, a coil block 2404, and the support 504 that together form an axially extending hollow portion, instead of the circuit block 2202, the electrode block 2203, the coil block 2204, and the support 104 (refer to FIG. 11 or the like) shown in FIG. 44.

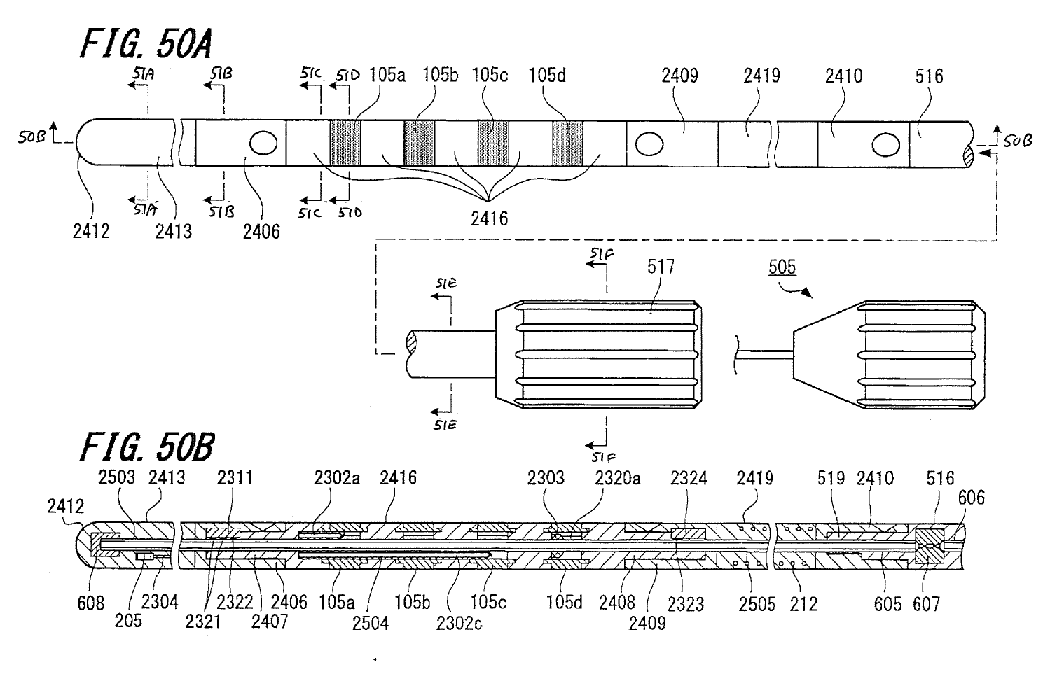

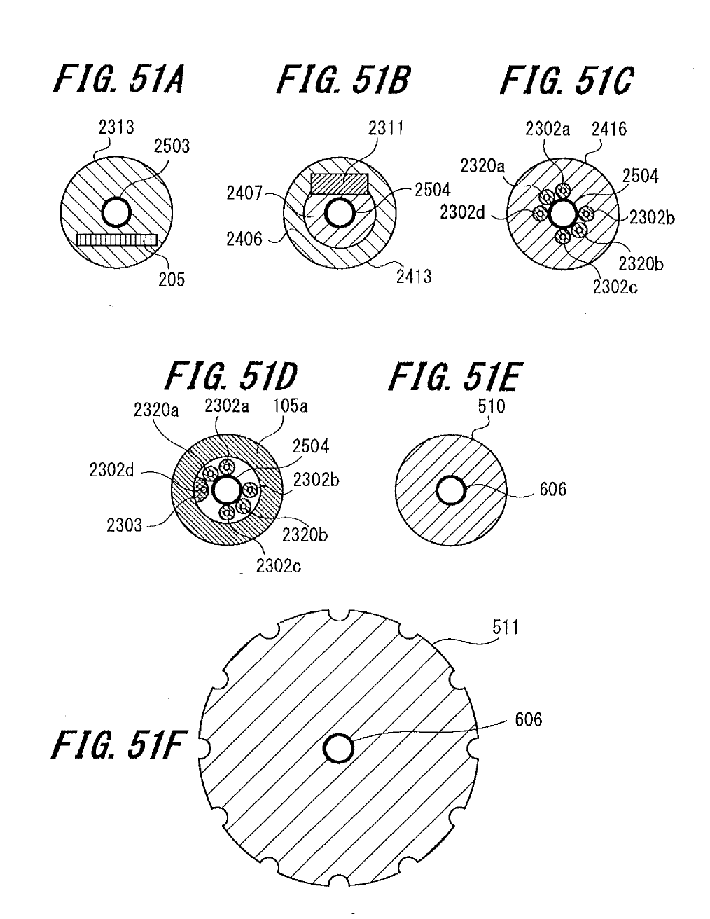

In the electric stimulator 2401, as shown in FIGS. 49A to 49D, when the respective blocks are connected together in the order of the circuit block 2402, the electrode block 2403, the coil block 2404, and the support 504, these blocks together form the lumen for a stylet that extends in the axial direction of the blocks. In this case, an opening for the stylet lumen is provided in the proximal end 511 of the electric stimulator 2201, and this opening extends up to the vicinity of the distal end 2412 of the electric stimulator 2401. In addition, the diameter of the lumen for a stylet is preferably approximately equal to or slightly longer than the outer diameter of the stylet 505. The internal configuration of the circuit block 2402, the electrode block 2403, and the coil block 2404 will be described below with reference to FIGS. 50A and 50B and FIGS. 51A to 51F. Since the internal structure of the support 504 has been described in FIGS. 13A and 13B, the detailed description thereof is omitted.

An eleventh embodiment of the electric stimulator is described below with reference to FIGS. 50A and 50B and FIGS. 51A to 51F. The internal configuration of the circuit block 2402 will first be described.

A pipe 2503 is made of materials that have biocompatibility, insulation, and pliability, for example, PTFE or ETFE, and possesses a hollow, substantially cylindrical shape in the axial direction. The external diameter of the pipe 2503 is preferably about 0.1 mm to 1 mm, and the internal diameter of the pipe 2503 is preferably approximately equal to or slightly greater than the outer diameter of the stylet 505 so that the stylet 505 can pass through the inside of the pipe 2503. One end (the end at the distal end 2412) of the pipe 2503 is connected to the receiving portion 608 described in FIG. 13B. The pipe 2503 and the receiving portion 608 are housed in and fixed to an outer layer part made up of a body 2413, and a connector part 2406 (refer to FIG. 50B).

The body 2413 is made of resin materials that have pliability and biocompatibility, for example, materials, such as silicone or polyurethane. The distal end 2412 of the body 2413 is substantially hemispherical, and the radius thereof is preferably within a range of about 0.5 mm to 1.5 mm. The portion of the body 2413 other than the distal end 2412 possesses a substantially cylindrical shape that is hollow in the axial direction. The external diameter of the hollow, substantially cylindrical portion is desirably in a range of about 1 to 3 mm, and the internal diameter is approximately equal to the external diameter of the receiving portion 608 or the pipe 2503, in order to house and fix the receiving portion 608 and the pipe 2503. The stimulation circuit 205 described in FIGS. 3B and 5, and the conducting wires 2304 that electrically connect the stimulation circuit 205 and the connector part 2406 are embedded in the body 2413, (refer to FIG. 51A).

The connector part 2406 is made of the same material as the body 2413, except for the electrical connecting portion 2311 that will be described below, and possesses a hollow, substantially cylindrical shape in the axial direction. The external diameter of the connector part 2406 is approximately equal to the external diameter of the body 2413, and the hollow portion of the connector part 2406 possesses almost the same shape and size as the outer shell of a connector part 2407 so that the connector part 2406 is connectable to the connector part 2407 of the electrode block 2403. The conducting wires 2304 and the electrical connecting portion 2311 are the same as those in the tenth embodiment, and so they are designated by the same reference numerals, and a detailed description is omitted.

Next, the internal configuration of the electrode block 2403 will be described.

The electrode block 2403 includes a pipe 2504 that is the same as the pipe 2503, except for the axial length thereof. The pipe 2504 is housed in and fixed to an outer layer part including the connector part 2407, a body 2416, the stimulation electrodes 105, and a connector part 2408 (refer to FIG. 50B).

The body 2416 is made of the same material as the body 2413 of the circuit block 2402, and possesses a hollow, substantially cylindrical shape in the axial direction. The external diameter of the body 2416 is equal to the external diameter of the hollow, substantially cylindrical portion of the body 2413 of the circuit block 2402, and the internal diameter of the body 2416 is approximately equal to the external diameter of the pipe 2504 in order to house and fix the pipe 2504. The four stimulation electrodes 105 are provided so as to be exposed at the surface of the body 2416, with portions of the body 2416 positioned between exposed axially adjacent stimulation electrodes 105.

One end of each of the conducting wires 2302a to 2302d is bonded to a respective one of the stimulation electrodes 105a to 105d with solder 2303 (refer to FIGS. 50B and 51D). The other end of each of the conducting wires 2302a to 2302d is electrically connected to the connector part 2407. In addition, locations other than the locations of the conducting wires 2302 that arc bonded with the solder 2303 and the locations of the conducting wires 2302 that are electrically connected to the connector part 2407 are insulated and coated with PTFE or ETFE and are completely embedded in the body 2416 (refer to FIG. 51C).

The connector part 2407 is formed of the same material as the body 2416, and is formed as a cutout part or reduced outer diameter part in which a level difference is provided from the external diameter of the substantially cylindrical body 2416 (refer to FIG. 51B). This cutout part extends a predetermined distance in the axial direction from a distal end 2417. Additionally, a total of the six connector pins 2321 and 2322 are exposed and arranged on the cutout part (refer to FIG. 49B). Moreover, a substantially cylindrical shape having an outer diameter equal to the external diameter of the pipe 2504 is formed on the axis of the connector part 2407 so as to house and fix the pipe 2504.

The connector part 2408 is made of the same material as the connector part 2407, and is formed in the same shape and size. The connector part 2408 is arranged such that the two connector pins 2323 are exposed onto a cutout part or reduced outer diameter part, differently from the connector part 2407 (refer to FIG. 49B). The connector pins 2321 to 2323 are the same as those in the tenth embodiment and are designated by the same reference numerals, and so a detailed descript of the connector pins 2321 to 2323 is not repeated. The internal configuration of the coil block 2404 (refer to FIG. 49C) will next be described.

The coil block 2404 includes a pipe 2505 that is the same as the pipes 2503 and 2504, except for the axial length thereof. The pipe 2505 is housed in and fixed to an outer layer part including a connector part 2409, a body 2419, and a connector part 2410 (refer to FIG. 50B).

The body 2419 is made of the same material as the body 2416 of the electrode block 2403. The body 2419 possesses a hollow, substantially cylindrical shape in the axial direction. The external diameter of the body 2419 is approximately equal to the external diameter of the body 2416 of the electrode block 2403, and the internal diameter of the body 2419 is approximately equal to the external diameter of the pipe 2505 in order to house and fix the pipe 2505. The coil portion 212 is formed so as to be wound in the axial direction and is embedded on the body 2419. In addition, the coil portion 212 is electrically connected to the connector part 2409 on a distal end 2420 (refer to FIG. 49C) of the body 2419.

The connector part 2409 is made of the same material as the body 2419 except for the electrical connecting portion 2324, and possesses a hollow, substantially cylindrical shape in the axial direction. The external diameter of the connector part 2409 is approximately equal to the external diameter of the body 2419, and the hollow portion of the connector part 2409 possesses almost the same shape and size as the outer shell of the connector part 2408 so that the connector part 2409 is connectable to the connector part 2408 of the electrode block 2403. Since the electrical connecting portion 2324 is the same as that described in the tenth embodiment, the electrical connecting portion 2324 is designated by the same reference numeral, and a detailed description of the electrical connecting portion 2324 is not repeated. The connector part 2410 is the same shape and size as the connector part 2409, and is made of the same material as the body 2419.

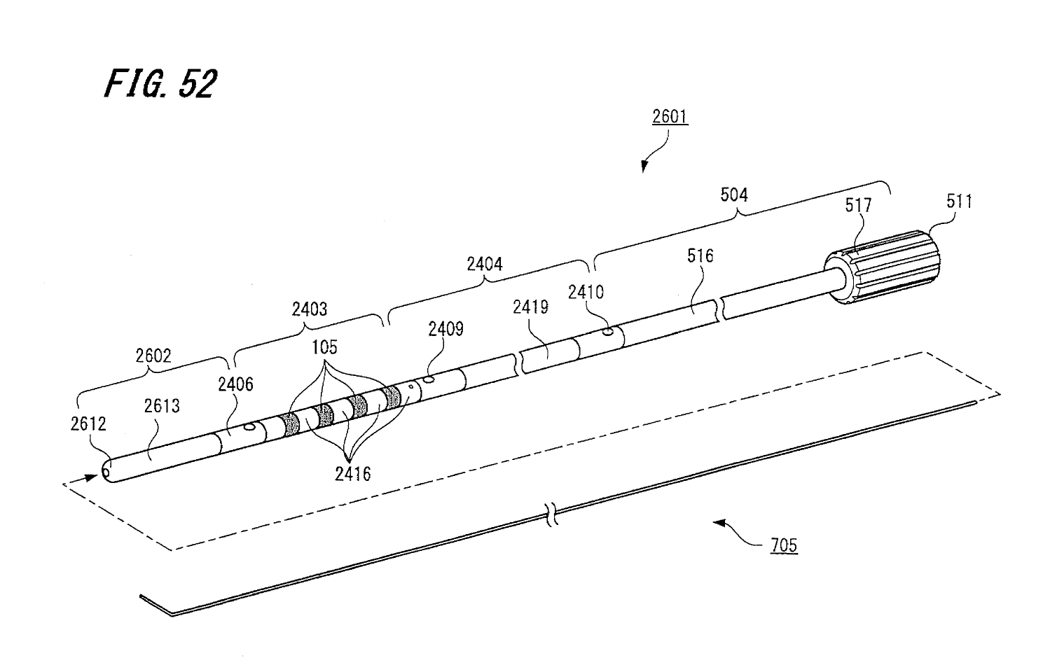

A twelfth embodiment of the electric stimulator is described next with reference to FIGS. 52 to 54. The electric stimulator 2601 according to the twelfth embodiment shown in FIGS. 52 to 54 is similar to the electric stimulator 2201 according to the tenth embodiment, but also includes a lumen for a guide wire. Features in this twelfth embodiment that are the same as features in earlier described embodiments are designated by common reference numerals, and a detailed description of such features is not repeated. The implantation procedure of the electric stimulator 2601 according to this embodiment is the same as the implantation procedure of the electric stimulator 701 according to the third embodiment described above, and so a detailed description of the implantation procedure is also not repeated.

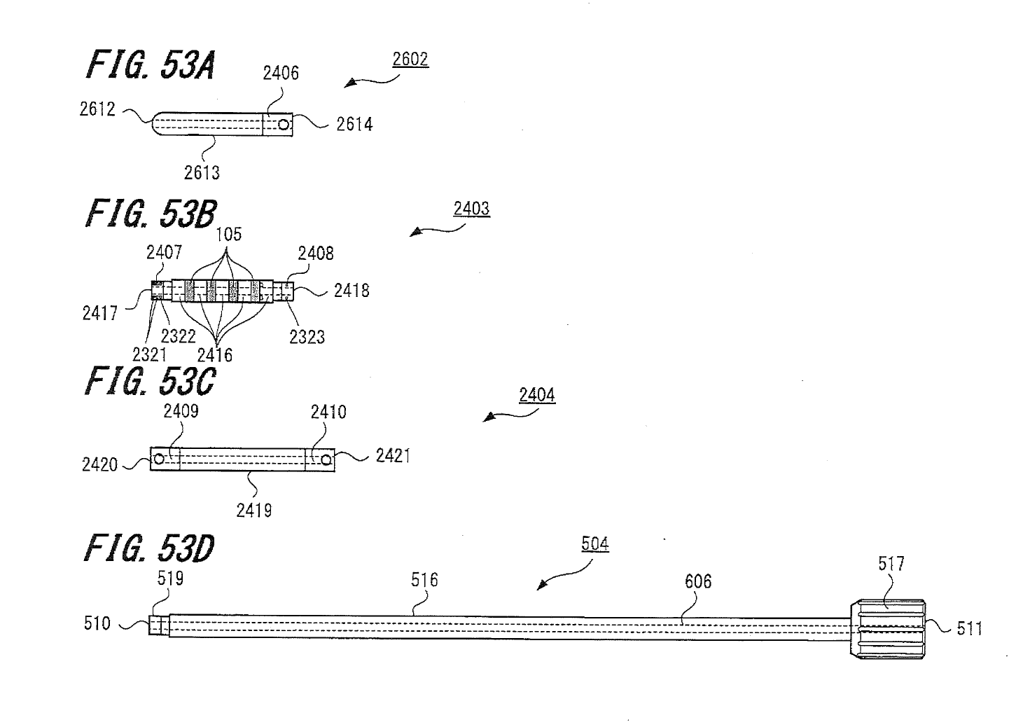

The general configuration of the electric stimulator related to the twelfth embodiment will be described with reference to FIG. 52 and FIGS. 53A to 53D.

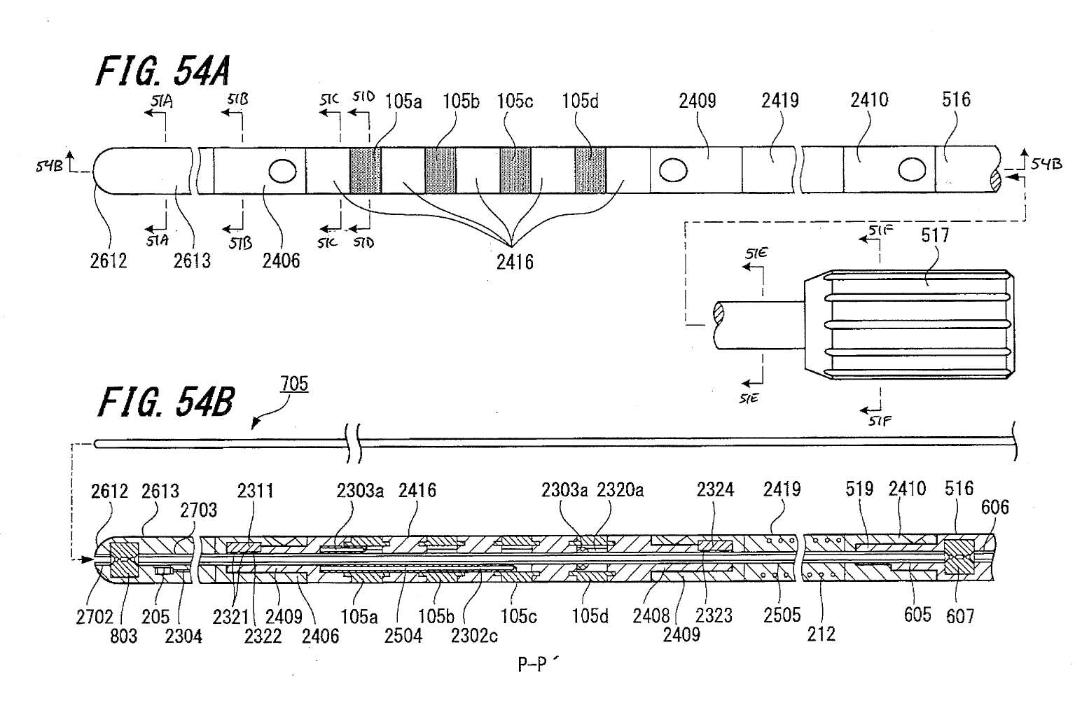

Similar to the above-described respective electric stimulators, the electric stimulator 2601 is formed in the shape of a rod (i.e., is rod-shaped), generates an electric stimulation signal, and stimulates nerves or the like in a living body by the stimulation signal. The electric stimulator 2601 has the axially extending lumen for a guide wire as mentioned above, and so the electric stimulator 2601 possesses a hollow, substantially cylindrical shape. The lumen for a guide wire is provided as a through hole that opens to the proximal end 511 and also opens to the distal end 2612. Therefore, the electric stimulator 2601 has a configuration in which the circuit block 2402 of the electric stimulator 2401 of the eleventh embodiment is replaced with a substantially cylindrical circuit block 2602 formed with a hollow portion extending throughout in the axial direction. The internal configuration of the circuit block 2602 will be described in FIGS. 54A and 54B.

The internal configuration of the electric stimulator according to the twelfth embodiment will be now described with reference to FIGS. 54A and 54B and FIGS. 51A to 51F. FIGS. 54A and 54B are enlarged views showing the electric stimulator according to the twelfth embodiment, and its axial internal structure.

FIGS. 51A to 51F, as described above, are cross-sectional views showing the internal structure at the noted axial locations, in the radial direction with respect to the axis of the electric stimulator 2401, according to the tenth embodiment, but these drawings are also cross-sectional views showing the internal structure in a radial direction with respect to the axis of the electric stimulator 2601 according to the twelfth embodiment.

The following description focuses primarily upon the internal configuration of the circuit block 2602 (refer to FIG. 53A). The circuit block 2602 includes pipes 2702 and 2703 which are the same as the pipes 2504 to 2505, except for the axial length thereof.

The valve 803 described in FIG. 17B is provided between the pipe 2702 and the pipe 2703 in the axial direction. When the guide wire 705 is inserted and removed via the valve 803, liquids, such as body fluids, are prevented from entering the insides of the circuit block 2602, the electrode block 2403 and the coil block 2404 from the hole provided in the distal end 2612 of the body 2613.

The pipes 2702 and 2703 and the valve 803 are housed in and fixed to an outer layer part made up of the body 2613, and the connector part 2406 (refer to FIG. 54B).

The body 2613 is made of, for example, materials, such as silicone or polyurethane, and has a substantially cylindrical hole in the distal end 2612. The inner diameter of this hole is approximately equal to the external diameter of the pipe 2702. In addition, the external diameter of the body 2613 is the same as that of the body 2413 (refer to FIGS. 50A and 50B) of the eleventh embodiment. The connector part 2406 is the same as that described in the eleventh embodiment, and so a detailed description of the connector part is not repeated.

As described above, in the tenth to twelfth embodiments, the electrode block is provided between the circuit block and the coil block. Thus, the circuit block and the coil block can be arranged to be separated or spaced apart from each other. Since the influence of electromagnetic waves on the stimulation circuit of the circuit block can be reduced when the electromagnetic waves for performing communication/electric supply from an external device (a controller outside of the body) are transmitted to the coil portion of the coil block, the probability of occurrence of malfunction, failure, or the like of the apparatus, is lowered.

The electrode block, the circuit block, and the support in the above-described embodiments are attachable and detachable, respectively, by the connectors. But the connectors may be omitted, with the electrode block and the circuit block integrated in advance, the circuit block and the support integrated in advance, or all the blocks integrated in advance. Additionally, though the rechargeable battery is used as a power source in the above-described respective embodiments, a primary battery may be used instead of the rechargeable battery, or a capacitor may be used instead of the rechargeable battery and the electric stimulator may be operated, always receiving electric supply from a controller outside the body.

In the above-described respective embodiments, when the electric stimulator is implanted into a living body, a form in which the electric stimulator passes through the epidural needle is used. However, it is possible to further improve the precision of the arrangement of the stimulation electrodes into a living body by passing the electric stimulator through the inside of a cannula and inserting the electric stimulator into the living body after the cannula having pliability is led through the epidural needle in advance to near a part to be stimulated.

In the above-described tenth to twelfth embodiments, the circuit block, the electrode block, the coil block, and the support are connected in order from the distal end side. However, even if the coil block, the electrode block, the circuit block, and the support are connected in that order from the distal end side, since the coil block and the circuit block are separated and spaced apart from each other, the effects described above regarding the tenth to twelfth embodiments can be realized.

In the above-described tenth to twelfth embodiments, the coil block includes only the coil portion. However, a portion of the charging section, for example, a rectifier circuit, may be built in the coil block, or a portion of the coil portion (except a coil) may be built in the circuit block.

The supports in the above-described seventh to twelfth embodiments can be substituted with the supports related to the above-described fourth to sixth embodiments.

In the above-described first to third and seventh to twelfth embodiments, the fixtures regarding the above-described fourth to sixth embodiments may be attached to the cut surface of the support.

The “stimulation circuit block” defined in the claims is comprised of the circuit block and the electrode block In the above-described embodiments.

The principles, preferred embodiments disclosed by way of example and other disclosed aspects of the electric stimulator have been described in the foregoing specification. However, the invention intended to be protected is not to be construed as limited to the particular embodiments disclosed. Further, the embodiments described herein are to be regarded as illustrative rather than restrictive. Variations and changes may be made by others, and equivalents employed, without departing from the spirit of the present invention. Accordingly, it is expressly intended that all such variations, changes and equivalents which fall within the spirit and scope of the present invention as defined in the claims, be embraced thereby.

What is claimed is:

1. A rod-shaped electric stimulator implantable in a living body, the electric stimulator comprising:
a stimulation circuit block possessing an outer surface, the stimulation circuit block including a stimulation electrode exposed at the outer surface of the stimulation circuit block to stimulate nerves or muscles in the living body during operational use when the stimulation electrode is positioned in the living body, the stimulation circuit block also including a stimulation circuit electrically connected to the stimulation electrode to apply a stimulation signal to the stimulation electrode so that the stimulation electrode stimulates nerves or muscles in the living body;
a support positioned proximally of the stimulation circuit block and connected to the stimulation circuit block, the support possessing a proximal portion positioned outside the living body during operational use when the stimulation electrode is positioned in the living body, the support also possessing a distal portion;
the proximal portion of the support being configured to be grasped by a user to maintain the stimulation electrode at an implantation position in the living body;
the stimulation circuit block and the support together being rod-shaped, the entire stimulation circuit block and at least the distal portion of the support being pliable so the stimulation circuit block and the distal portion of the support bend when moved inside the living body during operational use; and
the stimulation circuit block and the distal portion of the support possessing a shape and size permitting the stimulation circuit block and the distal portion of the support to be inserted into a duct of a tubular lead-in tool.

2. A rod-shaped electric stimulator implantable in a living body, the electric stimulator comprising:
a stimulation circuit block including a stimulation electrode that stimulates nerves or muscles in the living body and a stimulation circuit electrically connected to the stimulation electrode to apply a stimulation signal to the stimulation electrode;
a support connected to the stimulation circuit block to hold the stimulation electrode at an implantation position in the living body, the support possessing one end and an other end; and
the stimulation circuit block and the support, except for at least the one end of the support, possess a shape and size configured to be inserted into a duct of a tubular lead-in tool.

3. The electric stimulator according to claim 2, wherein the other end of the support includes a connector, and both ends of the stimulation circuit block include connectors connectable to the connector of the support.

4. The electric stimulator according to claim 2, wherein the stimulation circuit block includes an electrode block and a circuit block, the electrode block including the stimulation electrode which is positioned closer to a first axial end of the electrode block than an oppositely positioned second axial end of the electrode block, the circuit block also possessing first axial end and an oppositely positioned second axial end, the first and second axial ends of the electrode block both being connectable to and disconnectable from the first axial end of the circuit block

5. The electric stimulator according to claim 2, wherein the stimulation circuit block comprises an electrode block in which is located the stimulation electrode and a circuit block in which is located the stimulation circuit, the electrode block being separable from the circuit block.

6. The electric stimulator according to claim 5, wherein the electrode block and the circuit block each include a first end and a second end, the first end of the electrode block and the first end of the circuit block including a first connector configured to be connected to and disconnected from the connector of the support, the second end of the electrode block and the second end of the circuit block including second connectors configured to be connected to one another to electrically connect the stimulation electrode and the stimulation circuit.

7. The electric stimulator according to claim 6, wherein the first connector at the first end of the electrode block and the second connector at the second end of the circuit block are configured to be connected together, and when the first connector at the first end of the electrode block and the second connector at the second end of the circuit block are connected together, the stimulation electrode and the stimulation circuit are electrically connected together.

8. The electric stimulator according to claim 7, wherein the second connector at the second end of the electrode block is connectable to the connector of the support.

9. The electric stimulator according to claim 5, wherein the stimulation circuit block includes an electrode block and a circuit block, the electrode block including the stimulation electrode which is positioned closer to a first axial end of the electrode block than an oppositely positioned second axial end of the electrode block, the circuit block also possessing first axial end and an oppositely positioned second axial end, the first and second axial ends of the electrode block both being connectable to and disconnectable from the first axial end of the circuit block.

10. The electric stimulator according claim 6, further comprising an insertion block having a rounded first end and a second end, the second end of th4e insertion block including a connector configured to be detachably connected to both the first end of the electrode block and the second end of the electrode block.

11. The electric stimulator according to claim 2, wherein the support is devoid of an electric line, and the support possesses a plurality of spaced apart smaller outer dimension portions and a plurality of larger outer dimension portions each positioned between axially adjacent pairs of the smaller outer dimension portions, the smaller outer dimension portions being more easily cut than the larger outer dimension portions, the smaller outer dimension portions being configured to be cut to shorten the electric stimulator.

12. The electric stimulator according to claim 2, wherein the support possesses a lumen extending throughout the support from one end of the support to an opposite end of the support, and the stimulation circuit block possesses a lumen communicating with the lumen of the support.

13. The electric stimulator according to claim 2, wherein the support has a visually observable graduation for measuring how deep the electric stimulator is implanted in the living body.

14. The electric stimulator according to claim 2, further comprising a fixture connectable to the support to cover a cut surface of the support when the support is cut, the fixture being configured to be fixed to a tissue in the living body.

15. The electric stimulator according to claim 2, wherein the stimulation circuit block is substantially cylindrical.

16. The electric stimulator according to claim 15, wherein the support has substantially the same cylindrical shape as the stimulation circuit block.

17. The electric stimulator according to claim 2, wherein the stimulation circuit block further includes a power supply part that supplies electric power for generating a stimulation signal to the stimulation circuit.

18. The electric stimulator according to claim 17, wherein the power supply part comprises a coil configured to generate electric power by electromagnetic induction from the outside.

19. The electric stimulator according to claim 18, wherein the coil is wound so that coil and the stimulation circuit block are coaxial.

20. The electric stimulator according to claim 2, wherein the stimulation circuit block and the support are pliable to a bendable degree so that the stimulation circuit block and the support bend during use when inserted into a living body.

21. A method of stimulating nerves or muscles in a living body comprising:
inserting a distal end of a rod-shaped electric stimulator into a duct of a tubular lead-in tool that is positioned in an epidural space inside a living body, the electric stimulator comprising a stimulation electrode and a stimulation circuit electrically connected to the stimulation electrode, the stimulation electrode and the stimulation circuit together being connected to a support positioned proximally of the stimulation electrode and the stimulation circuit;
advancing the electric stimulator through the duct of the tubular lead-in tool by operating a portion of the support to position the stimulation electrode in the epidural space at a stimulation site; and
delivering an electric stimulation signal to the electric stimulator to electrically stimulate the stimulation site.
